SEQUENCE LISTING (4 sequences)

SEQ ID NO: 1 - DNA, 97 bp, synthetic construct
  misc_feature 1..97: note = Synthetic polynucleotide
  source 1..97: mol_type = other DNA
aagcttgaaagagagcaacaaagagagcaacaaagagagcaacaaagagagcaacaaagagagcaacaaagagagcaacaaagagagcaccccatgg

SEQ ID NO: 2 - DNA, 97 bp, synthetic construct
  misc_feature 1..97: note = Synthetic polynucleotide
  source 1..97: mol_type = other DNA
aagcttgaaggcggccaacaaggcggccaacaaggcggccaacaaggcggccaacaaggcggccaacaaggcggccaacaaggcggccaccccatgg

SEQ ID NO: 3 - DNA, 97 bp, synthetic construct
  misc_feature 1..97: note = Synthetic polynucleotide
  source 1..97: mol_type = other DNA
aagcttgaaccgccgcaacaaccgccgcaacaaccgccgcaacaaccgccgcaacaaccgccgcaacaaccgccgcaacaaccgccgcaccccatgg

SEQ ID NO: 4 - DNA, 97 bp, synthetic construct
  misc_feature 1..97: note = Synthetic polynucleotide
  source 1..97: mol_type = other DNA
aagcttgaacaacaacaacaacaacaacaacaacaacaacaacaacaacaacaacaacaacaacaacaacaacaacaacaacaacaacaccccatgg

(19) United States
(12) Patent Application Publication
(10) Pub. No.: US 2026/0345791 A1
(43) Pub. Date: Nov. 5, 2026

(54) EIF4A INHIBITORS AND USES THEREOF

(71) Applicant: The Regents of the University of California, Oakland, CA (US)

(72) Inventors: Kevan M. Shokat, San Francisco, CA (US); Kevin Lou, San Francisco, CA (US); Megan K. Moore, San Francisco, CA (US)

(21) Appl. No.: 18/995,023

(22) PCT Filed: Jul. 14, 2023
(86) PCT No.: PCT/US2023/070278

§ 371 Date: Jan. 15, 2025

Related U.S. Application Data
(60) Provisional application No. 63/389,571, filed on Jul. 15, 2022.

(51) Int. Cl. A61K 47/55; A61K 47/54

(52) U.S. Cl. CPC A61K 47/55; A61K 47/545

(57) ABSTRACT

Described herein, inter alia, are eIF4A inhibitors and uses thereof comprising a first eIF4A inhibitor attached to a second eIF4A inhibitor through a covalent linker. In preferred embodiments, the first eIF4A inhibitor and the second eIF4A inhibitor are the same and comprise Rocaglamide A, Zotatifin, Silvestrol, Pateamine A or Hippuristanol, and the linker comprises polyethylene glycol, polyethylene imine or polyethylene sulfide. Further provided a pharmaceutical composition comprising a pharmaceutically acceptable excipient and a compound and a method of treating cancer in a subject in need thereof, said method comprising administering to the subject in need thereof a therapeutically effective amount of a compound.

### CROSS-REFERENCES TO RELATED APPLICATIONS

This application claims the benefit of U.S. Provisional Application No. 63/389,571, filed Jul. 15, 2022, which is incorporated herein by reference in its entirety and for all purposes.

### REFERENCE TO AN ELECTRONIC SEQUENCE LISTING

The contents of the electronic sequence listing (048536-741001WO_Sequence_Listing_ST26.xml; Size: 6,426 bytes; and Date of Creation: Jul. 3, 2023) is hereby incorporated by reference in its entirety.

### BACKGROUND

The search for cell permeable drugs has conventionally been focused around low molecular weight, non-polar, and rigid chemical space. However, emerging therapeutic strategies employing flexibly linked chemical entities composed of more than one ligand necessitate exploration of new chemical space. A growing number of such large, flexible molecules are progressing through early discovery and into the clinic while violating traditional heuristics for cell permeability. This suggested to us that mechanisms apart from passive diffusion may be involved in these molecules' navigation through cell membranes. Disclosed herein, inter alia, are solutions to these and other problems in the art.

### BRIEF SUMMARY

In an aspect is provided a compound including a first eIF4A inhibitor attached to a second eIF4A inhibitor through a covalent linker.

In an aspect is provided a pharmaceutical composition including a compound described herein, or a pharmaceutically acceptable salt thereof, and a pharmaceutically acceptable excipient.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of modulating the level of activity of an eIF4A protein in a cell, the method including contacting the cell with an effective amount of a compound described herein, or a pharmaceutically acceptable salt thereof.

### BRIEF DESCRIPTION OF THE DRAWINGS

FIGS. 1A-1E. Longer linker length correlates with greater IFITM assistance in a series of linked rocaglamide analogs. FIG. 1A: Crystal structure of rocaglamide bound to eIF4A1 and polypurine RNA showing adjacent symmetry mates (PDB, 5ZC9). The distance between RocA molecules bound to adjacent eIF4A1 proteins is approximately 14 Å and the solvent exposed amide on RocA offers a handle for chemical modification. FIG. 1B: Chemical structures of select eIF4A1 inhibitors. FIGS. 1C-1D: Viability of K562 CRISPRi (FIG. 1C) or CRISPRa (FIG. 1D) cells expressing sgRNAs treated with BisRoc-1 or rocaglamide. Data represent means of three biological replicates; error bars denote SD. FIG. 1E: Chemical structures of a BisRoc linker length series (see also FIG. 2B).

FIGS. 2A-2C. IFITM proteins assist the cellular activity of diverse linked chemotypes. FIG. 2A: Heavy atom skeletons of compounds assessed for IFITM assistance (see also FIG. 3 for chemical structures). Compounds were categorized as non-linked chemotypes (compounds 3, 6, and 8; black), linked chemotypes with short linkers (compounds 10 and 13; light gray), or linked chemotypes with long linkers (compounds 15 and 17; medium gray). FIG. 2B: Chemical-genetic interaction map of inhibitors in FIG. 2A with IFITM1, IFTM2, and IFITM3. Potency, as measured by dose-response IC₅₀ in a cell viability assay (see also FIG. 1D for example source data), was normalized to that of non-sgRNA-expressing K562 CRISPRi or CRISPRa cells. Physicochemical properties, including molecular weight (MW) and number of rotatable bonds, with their respective traditional thresholds for drug-likeness are indicated (right). Data represent means of three biological replicates. FIG. 2C: Map of chemical space populated by 301 kinase inhibitors in clinical development (black), 2258 PROTACs reported in the literature (light gray), and 2 linked chemotypes described herein (medium gray). Boundaries represent traditional guidelines for drug-likeness.

FIGS. 3A-3B. Chemical structures of inhibitors assessed for IFITM assistance.

FIG. 4. Computed physicochemical properties of selected compounds described herein.

FIGS. 5A-5D. FIG. 5A: General synthetic scheme for the synthesis of BisRoc molecules from a commercially available rocaglate. FIG. 5B: RocA and BisRoc-1 were screened against a panel of ~300 cancer cell lines in a cell proliferation assay. FIG. 5C: Western blot of K562 treated 24 hr with DMSO or increasing doses of RocA, BisRoc-1, BisRoc-2, or BisRoc-3, blotting for oncogenes that are also known targets of RocA. FIG. 5D: mRNA transfection of luciferase reporters with model 5′-UTRs of varying sensitivities to RocA in HEK293T. Sequences shown:

(SEQ ID NO: 1)
AAGCTTGAA(AGAGAGCAACAA)₆AGAGAGCACCCCATGG;
(SEQ ID NO: 2)
AAGCTTGAA(GGCGGCCAACAA)₆GGCGGCCACCCCATGG;
(SEQ ID NO: 3)
AAGCTTGAA(CCGCCGCAACAA)₆CCGCCGCACCCCATGG;
(SEQ ID NO: 4)
AAGCTTGAA(CAACAACAACAA)₆CAACAACACCCCATGG.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

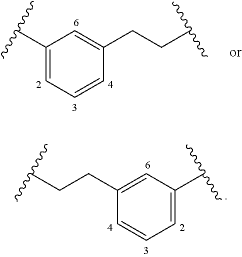 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NRC(NR′R″R″′)═NR″ ″, —NRC(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.1}, R^{L2.2}, R^{L3.2}, R^{L4.2}, R^{L5.2}, R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.1}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

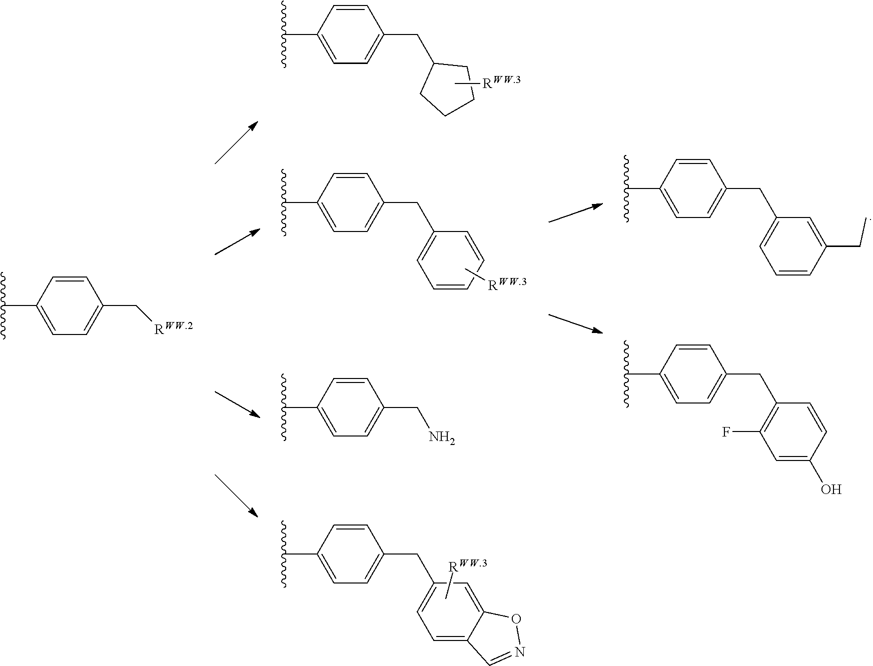

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “eIF4A” or “eukaryotic initiation factor-4A” refers to one or more of the family of proteins that function to unwind double-stranded RNA. In embodiments, eIF4A includes eIF4A1, eIF4A2, and eIF4A3. In embodiments, the term “eIF4A inhibitor” refers to an inhibitor of eIF4A. In embodiments, the eIF4A inhibitor is an eIF4A1 inhibitor. In embodiments, the eIF4A inhibitor is an eIF4A2 inhibitor. In embodiments, the eIF4A inhibitor is an eIF4A3 inhibitor.

The term “eIF4A1” or “eukaryotic initiation factor 4A-I” refers to a protein (including homologs, isoforms, and functional fragments thereof) that plays a role in unwinding double-stranded RNA. The term includes any recombinant or naturally-occurring form of eIF4A1 variants thereof that maintain eIF4A1 activity (e.g., within at least 30%, 40%, 50%, 60%, 70%, 80%, 90%, 95%, or 100% activity compared to wildtype eIF4A1). In embodiments, the eIF4A1 protein encoded by the eIF4A1 gene has the amino acid sequence set forth in or corresponding to Entrez 1973, UniProt P60842, RefSeq (protein) NP_001191439, or RefSeq (protein) NP_001407.1. In embodiments, the eIF4A1 gene has the nucleic acid sequence set forth in RefSeq (mRNA) NM_001204510 or RefSeq (mRNA) NM_001416.4. In embodiments, the amino acid sequence or nucleic acid sequence is the sequence known at the time of filing of the present application.

The term “eIF4A2” or “eukaryotic initiation factor 4A-II” refers to a protein (including homologs, isoforms, and functional fragments thereof) that plays a role in unwinding double-stranded RNA. The term includes any recombinant or naturally-occurring form of eIF4A2 variants thereof that maintain eIF4A2 activity (e.g., within at least 30%, 40%, 50%, 60%, 70%, 80%, 90%, 95%, or 100% activity compared to wildtype eIF4A2). In embodiments, the eIF4A2 protein encoded by the eIF4A2 gene has the amino acid sequence set forth in or corresponding to Entrez 1974, UniProt Q14240, or RefSeq (protein) NP_001958.2. In embodiments, the eIF4A2 gene has the nucleic acid sequence set forth in RefSeq (mRNA) NM_001967.4. In embodiments, the amino acid sequence or nucleic acid sequence is the sequence known at the time of filing of the present application.

The term “eIF4A3” or “eukaryotic initiation factor 4A-III” refers to a protein (including homologs, isoforms, and functional fragments thereof) that plays a role in unwinding double-stranded RNA. The term includes any recombinant or naturally-occurring form of eIF4A3 variants thereof that maintain eIF4A3 activity (e.g., within at least 30%, 40%, 50%, 60%, 70%, 80%, 90%, 95%, or 100% activity compared to wildtype eIF4A3). In embodiments, the eIF4A3 protein encoded by the eIF4A3 gene has the amino acid sequence set forth in or corresponding to Entrez 9775, UniProt P38919, or RefSeq (protein) NP_055555.1. In embodiments, the eIF4A3 gene has the nucleic acid sequence set forth in RefSeq (mRNA) NM_014740.4. In embodiments, the amino acid sequence or nucleic acid sequence is the sequence known at the time of filing of the present application.

The term “MYC” refers to one or more of the family of regulator genes and proto-oncogenes that code for transcription factors. In embodiments, MYC includes c-myc, 1-myc, and n-myc. In embodiments, the term “MYC-amplified cancer” refers to a cancer caused by overexpression of MYC. In embodiments, the MYC-amplified cancer is breast cancer. In embodiments, the MYC-amplified cancer is lung cancer (e.g., small-cell lung cancer). In embodiments, the MYC-amplified cancer is neuroblastoma.

The term “EGFR” or “epidermal growth factor” refers to a transmembrane protein (including homologs, isoforms, and functional fragments thereof) that is a receptor for members of the epidermal growth factor family of extracellular protein ligands. The term includes any recombinant or naturally-occurring form of EGFR variants thereof that maintain EGFR activity (e.g., within at least 30%, 40%, 50%, 60%, 70%, 80%, 90%, 95%, or 100% activity compared to wildtype EGFR). In embodiments, the EGFR protein encoded by the EGFR gene has the amino acid sequence set forth in or corresponding to Entrez 1956, UniProt P00533, RefSeq (protein) NP_005219.2, RefSeq (protein) NP_958439.1, RefSeq (protein) NP_958440.1, or RefSeq (protein) NP_958441.1. In embodiments, the amino acid sequence or nucleic acid sequence is the sequence known at the time of filing of the present application. In embodiments, the term “EGFR-amplified cancer” refers to a cancer caused by overexpression of EGFR. In embodiments, the EGFR-amplified cancer is breast cancer. In embodiments, the EGFR-amplified cancer is colorectal cancer. In embodiments, the EGFR-amplified cancer is gastric cancer.

The term “HER2” or “receptor tyrosine-protein kinase erbB-2” refers to a protein (including homologs, isoforms, and functional fragments thereof) in the human epidermal growth factor receptor family. The term includes any recombinant or naturally-occurring form of HER2 variants thereof that maintain HER2 activity (e.g., within at least 30%, 40%, 50%, 60%, 70%, 80%, 90%, 95%, or 100% activity compared to wildtype HER2). In embodiments, the HER2 protein encoded by the ERBB2 gene has the amino acid sequence set forth in or corresponding to Entrez 2064, UniProt P04626, RefSeq (protein) NP_001005862.1, RefSeq (protein) NP_001276865.1, RefSeq (protein) NP_001276867.1, or RefSeq (protein) NP_004439.2. In embodiments, the amino acid sequence or nucleic acid sequence is the sequence known at the time of filing of the present application. In embodiments, the term “HER2-amplified cancer” refers to a cancer caused by overexpression of HER2. In embodiments, the HER2-amplified cancer is breast cancer. In embodiments, the HER2-amplified cancer is gastric cancer.

The term “HER3” or “receptor tyrosine-protein kinase erbB-3” refers to a protein (including homologs, isoforms, and functional fragments thereof) in the human epidermal growth factor receptor family. The term includes any recombinant or naturally-occurring form of HER3 variants thereof that maintain HER3 activity (e.g., within at least 30%, 40%, 50%, 60%, 70%, 80%, 90%, 95%, or 100% activity compared to wildtype HER3). In embodiments, the HER3 protein encoded by the ERBB3 gene has the amino acid sequence set forth in or corresponding to Entrez 2065, UniProt P21860, RefSeq (protein) NP_001005915.1, or RefSeq (protein) NP_001973.2. In embodiments, the amino acid sequence or nucleic acid sequence is the sequence known at the time of filing of the present application. In embodiments, the term “HER3-amplified cancer” refers to a cancer caused by overexpression of HER3. In embodiments, the HER3-amplified cancer is breast cancer.

The term “FGFR1” or “fibroblast growth factor receptor 1” refers to a cell surface membrane receptor that possesses tyrosine kinase activity. The term includes any recombinant or naturally-occurring form of FGFR1 variants thereof that maintain FGFR1 activity (e.g., within at least 30%, 40%, 50%, 60%, 70%, 80%, 90%, 95%, or 100% activity compared to wildtype FGFR1). In embodiments, the FGFR1 protein encoded by the FGFR1 gene has the amino acid sequence set forth in or corresponding to Entrez 2260, UniProt P11362, RefSeq (protein) NP_001167534.1, RefSeq (protein) NP_001167535.1, RefSeq (protein) NP_001167536.1, RefSeq (protein) NP_001167537.1, or RefSeq (protein) NP_001167538.1. In embodiments, the amino acid sequence or nucleic acid sequence is the sequence known at the time of filing of the present application. In embodiments, the term “FGFR1-amplified cancer” refers to a cancer caused by overexpression of FGFR1. In embodiments, the FGFR1-amplified cancer is breast cancer. In embodiments, the FGFR1-amplified cancer is lung cancer (e.g., squamous cell lung carcinoma). In embodiments, the FGFR1-amplified cancer is pancreatic cancer.

The term “FGFR2” or “fibroblast growth factor receptor 2” refers to a cell surface membrane receptor that possesses tyrosine kinase activity. The term includes any recombinant or naturally-occurring form of FGFR2 variants thereof that maintain FGFR2 activity (e.g., within at least 30%, 40%, 50%, 60%, 70%, 80%, 90%, 95%, or 100% activity compared to wildtype FGFR2). In embodiments, the FGFR2 protein encoded by the FGFR2 gene has the amino acid sequence set forth in or corresponding to Entrez 2263, UniProt P21802, RefSeq (protein) NP_000132.3, RefSeq (protein) NP_001138385.1, RefSeq (protein) NP_001138386.1, RefSeq (protein) NP_001138387.1, or RefSeq (protein) NP_001138388.1. In embodiments, the amino acid sequence or nucleic acid sequence is the sequence known at the time of filing of the present application. In embodiments, the term “FGFR2-amplified cancer” refers to a cancer caused by overexpression of FGFR2. In embodiments, the FGFR2-amplified cancer is breast cancer. In embodiments, the FGFR2-amplified cancer is gastric cancer.

The term “RTK” or “receptor tyrosine kinase” refers to one or more of the family of cell surface receptors for many polypeptide growth factors, cytokines, and hormones. In embodiments, RTK includes EGF receptor, insulin receptor, PDGF receptor, VEGF receptor, FGF receptor, CCK receptor, NGF receptor, HGF receptor, Eph receptor, AXL receptor, TIE receptor, RYK receptor, DDR receptor, RET receptor, ROS receptor, LTK receptor, ROR receptor, MuSK receptor, and LMR receptor. In embodiments, the term “RTK-amplified cancer” refers to a cancer caused by overexpression of RTK. In embodiments, the term “mutated RTK cancer” refers to a cancer caused by a mutation of RTK. In embodiments, the RTK-amplified cancer is breast cancer. In embodiments, the RTK-amplified cancer is gastric cancer.

The term “K-Ras” refers to the protein that in humans is encoded by the KRAS gene. The K-Ras protein is a GTPase, which converts guanosine triphosphate to guanosine diphosphate. A mutation in the K-Ras protein (e.g., an amino acid substitution) can result in various malignancies (e.g., lung adenocarcinoma, pancreatic cancer, or colorectal cancer). The term includes any recombinant or naturally-occurring form of K-Ras variants thereof that maintain K-Ras activity (e.g., within at least 30%, 40%, 50%, 60%, 70%, 80%, 90%, 95%, or 100% activity compared to wildtype K-Ras). In embodiments, the K-Ras protein encoded by the KRAS gene has the amino acid sequence set forth in or corresponding to Entrez 3845, UniProt P01116, RefSeq (protein) NP_004976.2, or RefSeq (protein) NP_203524.1. In embodiments, the amino acid sequence or nucleic acid sequence is the sequence known at the time of filing of the present application. In embodiments, the term “mutated KRAS cancer” refers to a cancer caused by a mutation of KRAS. In embodiments, the mutated KRAS cancer is leukemia (e.g., acute myeloid leukemia or acute lymphoblastic leukemia). In embodiments, the mutated KRAS cancer is multiple myeloma. In embodiments, the mutated KRAS cancer is breast cancer. In embodiments, the mutated KRAS cancer is colorectal cancer. In embodiments, the mutated KRAS cancer is ovarian cancer. In embodiments, the mutated KRAS cancer is endometrial cancer. In embodiments, the mutated KRAS cancer is lung cancer (e.g., non-small cell lung cancer). In embodiments, the mutated KRAS cancer is pancreatic cancer. In embodiments, the mutated KRAS cancer is prostate cancer. In embodiments, the mutated KRAS cancer is skin cancer (e.g., squamous cell carcinoma).

The term “cyclin D1” refers to a protein (including homologs, isoforms, and functional fragments thereof) encoded by the CCND1 gene. The term includes any recombinant or naturally-occurring form of cyclin D1 variants thereof that maintain cyclin D1 activity (e.g., within at least 30%, 40%, 50%, 60%, 70%, 80%, 90%, 95%, or 100% activity compared to wildtype cyclin D1). In embodiments, the cyclin D1 protein encoded by the CCND1 gene has the amino acid sequence set forth in or corresponding to Entrez 595, UniProt P24385, or RefSeq (protein) NP_444284.1. In embodiments, the amino acid sequence or nucleic acid sequence is the sequence known at the time of filing of the present application. In embodiments, the term “cyclin D1-amplified cancer” refers to a cancer caused by overexpression of cyclin D1. In embodiments, the cyclin D1-amplified cancer is bladder cancer. In embodiments, the cyclin D1-amplified cancer is breast cancer. In embodiments, the cyclin D1-amplified cancer is esophageal cancer. In embodiments, the cyclin D1-amplified cancer is lung cancer.

The term “cyclin D2” refers to a protein (including homologs, isoforms, and functional fragments thereof) encoded by the CCND2 gene. The term includes any recombinant or naturally-occurring form of cyclin D2 variants thereof that maintain cyclin D2 activity (e.g., within at least 30%, 40%, 50%, 60%, 70%, 80%, 90%, 95%, or 100% activity compared to wildtype cyclin D2). In embodiments, the cyclin D2 protein encoded by the CCND2 gene has the amino acid sequence set forth in or corresponding to Entrez 894, UniProt P30279, or RefSeq (protein) NP_001750.1. In embodiments, the amino acid sequence or nucleic acid sequence is the sequence known at the time of filing of the present application. In embodiments, the term “cyclin D2-amplified cancer” refers to a cancer caused by overexpression of cyclin D2. In embodiments, the cyclin D2-amplified cancer is breast cancer. In embodiments, the cyclin D2-amplified cancer is gastric cancer.

The term “cyclin D3” refers to a protein (including homologs, isoforms, and functional fragments thereof) encoded by the CCND3 gene. The term includes any recombinant or naturally-occurring form of cyclin D3 variants thereof that maintain cyclin D3 activity (e.g., within at least 30%, 40%, 50%, 60%, 70%, 80%, 90%, 95%, or 100% activity compared to wildtype cyclin D3). In embodiments, the cyclin D3 protein encoded by the CCND3 gene has the amino acid sequence set forth in or corresponding to Entrez 896, UniProt P30281, RefSeq (protein) NP_001129489.1, RefSeq (protein) NP_001129597.1, RefSeq (protein) NP_001129598.1, RefSeq (protein) NP_001274356.1, RefSeq (protein) NP_001274363.1, or RefSeq (protein) NP_001751.1. In embodiments, the amino acid sequence or nucleic acid sequence is the sequence known at the time of filing of the present application. In embodiments, the term “cyclin D3-amplified cancer” refers to a cancer caused by overexpression of cyclin D3. In embodiments, the cyclin D3-amplified cancer is breast cancer. In embodiments, the cyclin D3-amplified cancer is skin cancer. In embodiments, the cyclin D3-amplified cancer is thyroid cancer.

The term “4EBP1” or “eukaryotic translation initiation factor 4E-binding protein 1” refers to a protein (including homologs, isoforms, and functional fragments thereof) encoded by the EIF4EBP1 gene. The term includes any recombinant or naturally-occurring form of 4EBP1 variants thereof that maintain 4EBP1 activity (e.g., within at least 30%, 40%, 50%, 60%, 70%, 80%, 90%, 95%, or 100% activity compared to wildtype 4EBP1). In embodiments, the 4EBP1 protein encoded by the EIF4EBP1 gene has the amino acid sequence set forth in or corresponding to Entrez 1978, UniProt Q13541, or RefSeq (protein) NP_004086.1. In embodiments, the amino acid sequence or nucleic acid sequence is the sequence known at the time of filing of the present application. In embodiments, the term “cancer with high phosphorylation of 4EBP1” refers to a cancer caused by high phosphorylation of 4EBP1. In embodiments, the cancer with high phosphorylation of 4EBP1 is renal cancer.

The term “4EBP2” or “eukaryotic translation initiation factor 4E-binding protein 2” refers to a protein (including homologs, isoforms, and functional fragments thereof) encoded by the EIF4EBP2 gene. The term includes any recombinant or naturally-occurring form of 4EBP2 variants thereof that maintain 4EBP2 activity (e.g., within at least 30%, 40%, 50%, 60%, 70%, 80%, 90%, 95%, or 100% activity compared to wildtype 4EBP2). In embodiments, the 4EBP2 protein encoded by the EIF4EBP2 gene has the amino acid sequence set forth in or corresponding to Entrez 1979, UniProt Q13542, or RefSeq (protein) NP_004087.1. In embodiments, the amino acid sequence or nucleic acid sequence is the sequence known at the time of filing of the present application. In embodiments, the term “cancer with high phosphorylation of 4EBP2” refers to a cancer caused by high phosphorylation of 4EBP2. In embodiments, the cancer with high phosphorylation of 4EBP2 is breast cancer (e.g., triple negative breast cancer).

The term “TSC1” or “tuberous sclerosis 1” refers to a protein (including homologs, isoforms, and functional fragments thereof) encoded by the TSC1 gene. The term includes any recombinant or naturally-occurring form of TSC1 variants thereof that maintain TSC1 activity (e.g., within at least 30%, 40%, 50%, 60%, 70%, 80%, 90%, 95%, or 100% activity compared to wildtype TSC1). In embodiments, the TSC1 protein encoded by the TSC1 gene has the amino acid sequence set forth in or corresponding to Entrez 7248, UniProt Q92574, RefSeq (protein) NP_000359.1, RefSeq (protein) NP_001155898.1, or RefSeq (protein) NP_001155899.1. In embodiments, the amino acid sequence or nucleic acid sequence is the sequence known at the time of filing of the present application. In embodiments, the term “cancer with loss of function of TSC1” refers to a cancer caused by loss of function of TSC1. In embodiments, the cancer with loss of function of TSC1 is bladder cancer. In embodiments, the cancer with loss of function of TSC1 is lung cancer. In embodiments, the cancer with loss of function of TSC1 is renal cancer. In embodiments, the cancer with loss of function of TSC1 is uterine cancer.

The term “TSC2” or “tuberous sclerosis 2” refers to a protein (including homologs, isoforms, and functional fragments thereof) encoded by the TSC2 gene. The term includes any recombinant or naturally-occurring form of TSC2 variants thereof that maintain TSC2 activity (e.g., within at least 30%, 40%, 50%, 60%, 70%, 80%, 90%, 95%, or 100% activity compared to wildtype TSC2). In embodiments, the TSC2 protein encoded by the TSC2 gene has the amino acid sequence set forth in or corresponding to Entrez 7249, UniProt P49815, RefSeq (protein) NP_000539.2, RefSeq (protein) NP_001070651.1, RefSeq (protein) NP_001107854.1, RefSeq (protein) NP_001305756.1, or RefSeq (protein) NP_001305758.1. In embodiments, the amino acid sequence or nucleic acid sequence is the sequence known at the time of filing of the present application. In embodiments, the term “cancer with loss of function of TSC2” refers to a cancer caused by loss of function of TSC2. In embodiments, the cancer with loss of function of TSC2 is liver cancer (e.g., hepatocellular carcinoma).

The term “PTEN” or “phosphatase and tensin homolog” refers to a protein (including homologs, isoforms, and functional fragments thereof) encoded by the PTEN gene. The term includes any recombinant or naturally-occurring form of PTEN variants thereof that maintain PTEN activity (e.g., within at least 30%, 40%, 50%, 60%, 70%, 80%, 90%, 95%, or 100% activity compared to wildtype PTEN). In embodiments, the PTEN protein encoded by the PTEN gene has the amino acid sequence set forth in or corresponding to Entrez 5728, UniProt P60484, RefSeq (protein) NP_000305.3, RefSeq (protein) NP_001291646.2, or RefSeq (protein) NP_001291647.1. In embodiments, the amino acid sequence or nucleic acid sequence is the sequence known at the time of filing of the present application. In embodiments, the term “cancer with loss of PTEN” refers to a cancer caused by loss of PTEN. In embodiments, the cancer with loss of PTEN is colorectal cancer. In embodiments, the cancer with loss of PTEN is prostate cancer.

### II. Compounds

In an aspect is provided a compound including a first eIF4A inhibitor attached to a second eIF4A inhibitor through a covalent linker.

In embodiments, the first eIF4A inhibitor and the second eIF4A inhibitor are the same.

In embodiments, the first eIF4A inhibitor and the second eIF4A inhibitor are a Rocaglate. In embodiments, the first eIF4A inhibitor and the second eIF4A inhibitor are Rocaglamide A. In embodiments, the first eIF4A inhibitor and the second eIF4A inhibitor are Zotatifin. In embodiments, the first eIF4A inhibitor and the second eIF4A inhibitor are Silvestrol. In embodiments, the first eIF4A inhibitor and the second eIF4A inhibitor are Pateamine A. In embodiments, the first eIF4A inhibitor and the second eIF4A inhibitor are Hippuristanol. In embodiments, the first eIF4A inhibitor and the second eIF4A inhibitor are a compound as described in Ernst, J. T. et al., J. Med. Chem. 2020, 63, 5879-5955, which is herein incorporated by reference in its entirety for all purposes. In embodiments, the first eIF4A inhibitor and the second eIF4A inhibitor are a compound as described in Naineni, S. K. et al., Cell Chem. Biol. 2021, 28, 825-834, which is herein incorporated by reference in its entirety for all purposes. In embodiments, the first eIF4A inhibitor and the second eIF4A inhibitor are a compound as described in Bordeleau, M.-E. et al., Nature Chem. Biol. 2006, 2, 213-220, which is herein incorporated by reference in its entirety for all purposes. In embodiments, the first eIF4A inhibitor and the second eIF4A inhibitor are a compound as described in Iwasaki, S. et al., Molecular Cell 2019, 73, 738-748, which is herein incorporated by reference in its entirety for all purposes. In embodiments, the first eIF4A inhibitor and the second eIF4A inhibitor are a compound as described in Iwasaki, S. et al., Nature 2016, 534, 558-561, which is herein incorporated by reference in its entirety for all purposes. In embodiments, the first eIF4A inhibitor and the second eIF4A inhibitor are a compound as described in Pelletier, J. et al., Cancer Res. 2015, 75, 250-263, which is herein incorporated by reference in its entirety for all purposes. In embodiments, the first eIF4A inhibitor and the second eIF4A inhibitor are a compound as described in Chu, J. et al., Cell Reports 2020, 30, 2481-2488, which is herein incorporated by reference in its entirety for all purposes. In embodiments, the first eIF4A inhibitor and the second eIF4A inhibitor are a compound as described in WO 2017/091585, WO 2018/218072, and WO 2021/195128, which are herein incorporated by reference in their entirety for all purposes.

In embodiments, the first eIF4A inhibitor and the second eIF4A inhibitor are a monovalent form of formula (I):

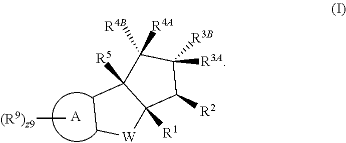

Ring A is aryl (e.g., C₆-C₁₀ or phenyl) or heteroaryl (e.g., 5 to 10 membered, 5 to 9 membered, or 5 to 6 membered).

W is CR⁶R⁷, O, S, NR⁸, C(O), C═CR⁶R⁷, N(CO)R⁸, S(O), or S(O)₂.

R¹ and R² are independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3A}, R^{3B}, R^{4A}, R^{4B}, R⁵, R⁶, and R⁷ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3A} and R^{3B}, or R^{4A} and R^{4B} independently combine to form oxo or substituted or unsubstituted alkenyl (e.g., C₂-C₈, C₂-C₆, or C₂-C₄), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered).

R² and R^{3A}, R^{3A} and R^{4A}, R^{3B} and R^{4B}, or R^{4A} and R⁵, together with the carbon atom to which they are attached, form a substituted or unsubstituted cycloalkyl (e.g., C₃-C₈, C₃-C₆, C₄-C₆, or C₅-C₆) or substituted or unsubstituted heterocycloalkyl (e.g., 3 to 8 membered, 3 to 6 membered, 4 to 6 membered, 4 to 5 membered, or 5 to 6 membered).

R⁸ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —N^{9C}NR^{9A}R^{9B}, —ONR^{9A}R^{9B}, —NHC(O)NR^{9A}R^{9B}, —N(O)ₘ₉, —NR^{9A}R^{9B}, —C(O)R^{9C}, —C(O)OR^{9C}, —C(O)NR^{9A}R^{9B}, —OR^{9D}, —SR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{9A} and R^{9B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X⁹ is independently —F, —Cl, —Br, or —I.

The symbol n9 is an integer from 0 to 4.

The symbols m9 and v9 are independently 1 or 2.

The symbol z9 is an integer from 0 to 4.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, R^{3A} is hydrogen. In embodiments, R^{3A} is halogen. In embodiments, R^{3A} is —F. In embodiments, R^{3A} is —Cl. In embodiments, R^{3A} is —Br. In embodiments, R^{3A} is —I. In embodiments, R^{3A} is —CCl₃. In embodiments, R^{3A} is —CBr₃. In embodiments, R^{3A} is —CF₃. In embodiments, R^{3A} is —CI₃. In embodiments, R^{3A} is —CH₂C₁. In embodiments, R^{3A} is —CH₂Br. In embodiments, R^{3A} is —CH₂F. In embodiments, R^{3A} is —CH₂I. In embodiments, R^{3A} is —CHCl₂. In embodiments, R^{3A} is —CHBr₂. In embodiments, R^{3A} is —CHF₂. In embodiments, R^{3A} is —CHI₂. In embodiments, R^{3A} is —CN. In embodiments, R^{3A} is —OH. In embodiments, R^{3A} is —NH₂. In embodiments, R^{3A} is —COOH. In embodiments, R^{3A} is —CONH₂. In embodiments, R^{3A} is —C(O)N(CH₃)₂. In embodiments, R^{3A} is —NO₂. In embodiments, R^{3A} is —SH. In embodiments, R^{3A} is —SO₃H. In embodiments, R^{3A} is —OSO₃H. In embodiments, R^{3A} is —SO₂NH₂. In embodiments, R^{3A} is —NHNH₂. In embodiments, R^{3A} is —ONH₂. In embodiments, R^{3A} is —NHC(O)NHNH₂. In embodiments, R^{3A} is —NHC(O)NH₂. In embodiments, R^{3A} is —NHSO₂H. In embodiments, R^{3A} is —NHC(O)H. In embodiments, R^{3A} is —NHC(O)OH. In embodiments, R^{3A} is —NHOH. In embodiments, R^{3A} is —OCCl₃. In embodiments, R^{3A} is —OCBr₃. In embodiments, R^{3A} is —OCF₃. In embodiments, R^{3A} is —OCI₃. In embodiments, R^{3A} is —OCH₂Cl. In embodiments, R^{3A} is —OCH₂Br. In embodiments, R^{3A} is —OCH₂F. In embodiments, R^{3A} is —OCH₂I. In embodiments, R^{3A} is —OCHCl₂. In embodiments, R^{3A} is —OCHBr₂. In embodiments, R^{3A} is —OCHF₂. In embodiments, R^{3A} is —OCHI₂. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl. In embodiments, R^{3A} is unsubstituted 2 to 4 membered heteroalkyl. In embodiments, R^{3A} is unsubstituted methoxy. In embodiments, R^{3A} is unsubstituted ethoxy. In embodiments, R^{3A} is unsubstituted propoxy. In embodiments, R^{3A} is unsubstituted n-propoxy. In embodiments, R^{3A} is unsubstituted isopropoxy. In embodiments, R^{3A} is unsubstituted butoxy. In embodiments, R^{3A} is unsubstituted n-butoxy. In embodiments, R^{3A} is unsubstituted isobutoxy. In embodiments, R^{3A} is unsubstituted tert-butoxy.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, R^{3B} is hydrogen. In embodiments, R^{3B} is halogen. In embodiments, R^{3B} is —F. In embodiments, R^{3B} is —Cl. In embodiments, R^{3B} is —Br. In embodiments, R^{3B} is —I. In embodiments, R^{3B} is —CCl₃. In embodiments, R^{3B} is —CBr₃. In embodiments, R^{3B} is —CF₃. In embodiments, R^{3B} is —CI₃. In embodiments, R^{3B} is —CH₂C₁. In embodiments, R^{3B} is —CH₂Br. In embodiments, R^{3B} is —CH₂F. In embodiments, R^{3B} is —CH₂I. In embodiments, R^{3B} is —CHCl₂. In embodiments, R^{3B} is —CHBr₂. In embodiments, R^{3B} is —CHF₂. In embodiments, R^{3B} is —CHI₂. In embodiments, R^{3B} is —CN. In embodiments, R^{3B} is —OH. In embodiments, R^{3B} is —NH₂. In embodiments, R^{3B} is —COOH. In embodiments, R^{3B} is —C(O)NH₂ or —C(O)N(CH₃)₂. In embodiments, R^{3B} is —CONH₂. In embodiments, R^{3B} is —C(O)N(CH₃)₂. In embodiments, R^{3B} is —NO₂. In embodiments, R^{3B} is —SH. In embodiments, R^{3B} is —SO₃H. In embodiments, R^{3B} is —OSO₃H. In embodiments, R^{3B} is —SO₂NH₂. In embodiments, R^{3B} is —NHNH₂. In embodiments, R^{3B} is —ONH₂. In embodiments, R^{3B} is —NHC(O)NHNH₂. In embodiments, R^{3B} is —NHC(O)NH₂. In embodiments, R^{3B} is —NHSO₂H. In embodiments, R^{3B} is —NHC(O)H. In embodiments, R^{3B} is —NHC(O)OH. In embodiments, R^{3B} is —NHOH. In embodiments, R^{3B} is —OCCl₃. In embodiments, R^{3B} is —OCBr₃. In embodiments, R^{3B} is —OCF₃. In embodiments, R^{3B} is —OCI₃. In embodiments, R^{3B} is —OCH₂Cl. In embodiments, R^{3B} is —OCH₂Br. In embodiments, R^{3B} is —OCH₂F. In embodiments, R^{3B} is —OCH₂I. In embodiments, R^{3B} is —OCHCl₂. In embodiments, R^{3B} is —OCHBr₂. In embodiments, R^{3B} is —OCHF₂. In embodiments, R^{3B} is —OCHI₂. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl. In embodiments, R^{3B} is —C(O)NH₂ or substituted or unsubstituted 2 to 6 membered heteroalkyl. In embodiments, R^{3B} is substituted or unsubstituted 2 to 6 membered heteroalkyl. In embodiments, R^{3B} is unsubstituted 2 to 4 membered heteroalkyl. In embodiments, R^{3B} is unsubstituted methoxy. In embodiments, R^{3B} is unsubstituted ethoxy. In embodiments, R^{3B} is unsubstituted propoxy. In embodiments, R^{3B} is unsubstituted n-propoxy. In embodiments, R^{3B} is unsubstituted isopropoxy. In embodiments, R^{3B} is unsubstituted butoxy. In embodiments, R^{3B} is unsubstituted n-butoxy. In embodiments, R^{3B} is unsubstituted isobutoxy. In embodiments, R^{3B} is unsubstituted tert-butoxy.

In embodiments, the compound has the formula:

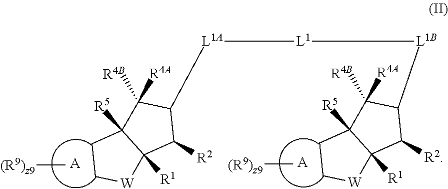

Ring A, W, R¹, R², R^{4A}, R^{4B}, R⁵, R⁹, and z9 are as described herein, including in embodiments.

L¹ is the covalent linker.

L^{1A} is a bond, —C(O)—, —C(O)O—, —OC(O)—, —O—, —S—, —NR^{10A}—, —C(O)NR^{10A}—, —NR^{10A}C(O)—, —NR^{10A}C(O)O—, —OC(O)NR^{10A}—, —NR^{10A}C(O)NR^{10A}—, —NR^{10A}C(NH)NR^{10A}—, —S(O)₂—, —NR^{10A}S(O)₂—, —S(O)₂NR^{10A}—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L^{1B} is a bond, —C(O)—, —C(O)O—, —OC(O)—, —O—, —S—, —NR^{10B}—, —C(O)NR^{10B}—, —NR^{10B}C(O)—, —NR^{10B}C(O)O—, —OC(O)NR^{10B}—, —NR^{10B}C(O)NR^{10B}—, —NR^{10B}C(NH)NR^{10B}—, —S(O)₂—, —NR^{10B}S(O)₂—, —S(O)₂NR^{10B}—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

Each R^{10A} and R^{10B} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted L^{1A} (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L^{1A} is substituted with a plurality of groups selected from substituent groups, size-limited substituent groups, and lower substituent groups; each substituent group, size-limited substituent group, and/or lower substituent group may optionally be different. In embodiments, when L^{1A} is substituted, it is substituted with at least one substituent group. In embodiments, when L^{1A} is substituted, it is substituted with at least one size-limited substituent group. In embodiments, when L^{1A} is substituted, it is substituted with at least one lower substituent group.

In embodiments, L^{1A} is a bond. In embodiments, L^{1A} is —C(O)—. In embodiments, L^{1A} is —C(O)O—. In embodiments, L^{1A} is —OC(O)—. In embodiments, L^{1A} is —O—. In embodiments, L^{1A} is —S—. In embodiments, L^{1A} is —NR^{10A}—. In embodiments, L^{1A} is —NH—. In embodiments, L^{1A} is —C(O)NR^{10A}—. In embodiments, L^{1A} is —C(O)NH—. In embodiments, L^{1A} is —NR^{10A}C(O)—. In embodiments, L^{1A} is —NHC(O)—. In embodiments, L^{1A} is —NR^{10A}C(O)O—. In embodiments, L^{1A} is —NHC(O)O—. In embodiments, L^{1A} is —OC(O)NR^{10A}—. In embodiments, L^{1A} is —OC(O)NH—. In embodiments, L^{1A} is —NR^{10A}C(O)NR^{10A}—. In embodiments, L^{1A} is —NHC(O)NH—. In embodiments, L^{1A} is —NR^{10A}C(NH)NR^{10A}—. In embodiments, L^{1A} is —NHC(NH)NH—. In embodiments, L^{1A} is —S(O)₂—. In embodiments, L^{1A} is —NR^{10A}S(O)₂—. In embodiments, L^{1A} is —NHS(O)₂—. In embodiments, L^{1A} is —S(O)₂NR^{10A}—. In embodiments, L^{1A} is —S(O)₂NH—. In embodiments, L^{1A} is —C(O)NR^{10A}— or substituted or unsubstituted 2 to 6 membered heteroalkylene. In embodiments, L^{1A} is —C(O)NR^{10A}CH₂—. In embodiments, L^{1A} is —C(O)NHCH₂—. In embodiments, L^{1A} is —CH₂NHC(O)—.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, R^{10A} is independently hydrogen or unsubstituted C₁-C₄ alkyl. In embodiments, R^{10A} is independently hydrogen. In embodiments, R^{10A} is independently unsubstituted C₁-C₄ alkyl. In embodiments, R^{10A} is independently unsubstituted methyl. In embodiments, R^{10A} is independently unsubstituted ethyl. In embodiments, R^{10A} is independently unsubstituted propyl. In embodiments, R^{10A} is independently unsubstituted n-propyl. In embodiments, R^{10A} is independently unsubstituted isopropyl. In embodiments, R^{10A} is independently unsubstituted butyl. In embodiments, R^{10A} is independently unsubstituted n-butyl. In embodiments, R^{10A} is independently unsubstituted isobutyl. In embodiments, R^{10A} is independently unsubstituted tert-butyl.

In embodiments, a substituted L^{1B} (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L^{1B} is substituted with a plurality of groups selected from substituent groups, size-limited substituent groups, and lower substituent groups; each substituent group, size-limited substituent group, and/or lower substituent group may optionally be different. In embodiments, when L^{1B} is substituted, it is substituted with at least one substituent group. In embodiments, when L^{1B} is substituted, it is substituted with at least one size-limited substituent group. In embodiments, when L^{1B} is substituted, it is substituted with at least one lower substituent group.

In embodiments, L^{1B} is a bond. In embodiments, L^{1B} is —C(O)—. In embodiments, L^{1B} is —C(O)O—. In embodiments, L^{1B} is —OC(O)—. In embodiments, L^{1B} is —O—. In embodiments, L^{1B} is —S—. In embodiments, L^{1B} is —NR^{10B}—. In embodiments, L^{1B} is —NH—. In embodiments, L^{1B} is —C(O)NR^{10B}—. In embodiments, L^{1B} is —C(O)NH—. In embodiments, L^{1B}is —NR^{10B}C(O)—. In embodiments, L^{1B} is —NHC(O)—. In embodiments, L^{1B} is —NR^{10B}C(O)O—. In embodiments, L^{1B} is —NHC(O)O—. In embodiments, L^{1B} is —OC(O)NR^{10B}—. In embodiments, L^{1B} is —OC(O)NH—. In embodiments, L^{1B} is —NR^{10B}C(O)NR^{10B}—. In embodiments, L^{1B} is —NHC(O)NH—. In embodiments, L^{1B} is —NR^{10B}C(NH)NR^{10B}—. In embodiments, L^{1B} is —NHC(NH)NH—. In embodiments, L^{1B} is —S(O)₂—. In embodiments, L^{1B} is —NR^{10B}S(O)₂—. In embodiments, L^{1B} is —NHS(O)₂—. In embodiments, L^{1B} is —S(O)₂NR^{10A}—. In embodiments, L^{1B} is —S(O)₂NH—. In embodiments, L^{1B} is —NR^{10B}C(O)— or substituted or unsubstituted 2 to 6 membered heteroalkylene. In embodiments, L^{1B} is —CH₂NR^{10A}C(O)—. In embodiments, L^{1B} is —CH₂NHC(O)—. In embodiments, L^{1B} is —C(O)NHCH₂—.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, R^{10B} is independently hydrogen or unsubstituted C₁-C₄ alkyl. In embodiments, R^{10B} is independently hydrogen. In embodiments, R^{10B} is independently unsubstituted C₁-C₄ alkyl. In embodiments, R^{10B} is independently unsubstituted methyl. In embodiments, R^{10B} is independently unsubstituted ethyl. In embodiments, R^{10B} is independently unsubstituted propyl. In embodiments, R^{10B} is independently unsubstituted n-propyl. In embodiments, R^{10B} is independently unsubstituted isopropyl. In embodiments, R^{10B} is independently unsubstituted butyl. In embodiments, R^{10B} is independently unsubstituted n-butyl. In embodiments, R^{10B} is independently unsubstituted isobutyl. In embodiments, R^{10B} is independently unsubstituted tert-butyl.

In embodiments, Ring A is phenyl or a 5 to 6 membered heteroaryl. In embodiments, Ring A is phenyl. In embodiments, Ring A is a 5 to 6 membered heteroaryl. In embodiments, Ring A is a pyridyl. In embodiments, Ring A is a pyrazinyl. In embodiments, Ring A is a pyrimidinyl. In embodiments, Ring A is a pyridazinyl. In embodiments, Ring A is a furanyl. In embodiments, Ring A is a thienyl. In embodiments, Ring A is a pyrrolyl. In embodiments, Ring A is a pyrazolyl. In embodiments, Ring A is an imidazolyl. In embodiments, Ring A is an oxazolyl. In embodiments, Ring A is an isoxazolyl. In embodiments, Ring A is a thiazolyl.

In embodiments, W is O, S, NH, or C(O). In embodiments, W is O. In embodiments, W is S. In embodiments, W is NH. In embodiments, W is C(O).

In embodiments, a substituted R¹ (e.g., substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is a substituted or unsubstituted phenyl. In embodiments, R¹ is a substituted phenyl. In embodiments, R¹ is an unsubstituted phenyl. In embodiments, R¹ is a methoxy-substituted phenyl. In embodiments, R¹ is

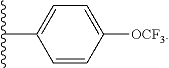 [SMILES: *c1ccc(OC(F)(F)F)cc1]

In embodiments, R¹ is a cyano-substituted phenyl. In embodiments, R¹ is

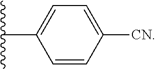 [SMILES: *c1ccc(C#N)cc1]

In embodiments, a substituted R² (e.g., substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is a substituted or unsubstituted phenyl. In embodiments, R² is a substituted phenyl. In embodiments, R² is an unsubstituted phenyl.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, R^{4A} is hydrogen. In embodiments, R^{4A} is halogen. In embodiments, R^{4A} is —F. In embodiments, R^{4A} is —Cl. In embodiments, R^{4A} is —Br. In embodiments, R^{4A} is —I. In embodiments, R^{4A} is —CCl₃. In embodiments, R^{4A} is —CBr₃. In embodiments, R^{4A} is —CF₃. In embodiments, R^{4A} is —CI₃. In embodiments, R^{4A} is —CH₂C₁. In embodiments, R^{4A} is —CH₂Br. In embodiments, R^{4A} is —CH₂F. In embodiments, R^{4A} is —CH₂I. In embodiments, R^{4A} is —CHCl₂. In embodiments, R^{4A} is —CHBr₂. In embodiments, R^{4A} is —CHF₂. In embodiments, R^{4A} is —CHI₂. In embodiments, R^{4A} is —CN. In embodiments, R^{4A} is —OH. In embodiments, R^{4A} is —NH₂. In embodiments, R^{4A} is —COOH. In embodiments, R^{4A} is —CONH₂. In embodiments, R^{4A} is —NO₂. In embodiments, R^{4A} is —SH. In embodiments, R^{4A} is —SO₃H. In embodiments, R^{4A} is —OSO₃H. In embodiments, R^{4A} is —SO₂NH₂. In embodiments, R^{4A} is —NHNH₂. In embodiments, R^{4A} is —ONH₂. In embodiments, R^{4A} is —NHC(O)NHNH₂. In embodiments, R^{4A} is —NHC(O)NH₂. In embodiments, R^{4A} is —NHSO₂H. In embodiments, R^{4A} is —NHC(O)H. In embodiments, R^{4A} is —NHC(O)OH. In embodiments, R^{4A} is —NHOH. In embodiments, R^{4A} is —OCCl₃. In embodiments, R^{4A} is —OCBr₃. In embodiments, R^{4A} is —OCF₃. In embodiments, R^{4A} is —OCI₃. In embodiments, R^{4A} is —OCH₂Cl. In embodiments, R^{4A} is —OCH₂Br. In embodiments, R^{4A} is —OCH₂F. In embodiments, R^{4A} is —OCH₂I. In embodiments, R^{4A} is —OCHCl₂. In embodiments, R^{4A} is —OCHBr₂. In embodiments, R^{4A} is —OCHF₂. In embodiments, R^{4A} is —OCHI₂. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl. In embodiments, R^{4A} is unsubstituted 2 to 4 membered heteroalkyl. In embodiments, R^{4A} is unsubstituted methoxy. In embodiments, R^{4A} is unsubstituted ethoxy. In embodiments, R^{4A} is unsubstituted propoxy. In embodiments, R^{4A} is unsubstituted n-propoxy. In embodiments, R^{4A} is unsubstituted isopropoxy. In embodiments, R^{4A} is unsubstituted butoxy. In embodiments, R^{4A} is unsubstituted n-butoxy. In embodiments, R^{4A} is unsubstituted isobutoxy. In embodiments, R^{4A} is unsubstituted tert-butoxy.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, R^{4B} is hydrogen. In embodiments, R^{4B} is halogen. In embodiments, R^{4B} is —F. In embodiments, R^{4B} is —Cl. In embodiments, R^{4B} is —Br. In embodiments, R^{4B} is —I. In embodiments, R^{4B} is —CCl₃. In embodiments, R^{4B} is —CBr₃. In embodiments, R^{4B} is —CF₃. In embodiments, R^{4B} is —CI₃. In embodiments, R^{4B} is —CH₂C₁. In embodiments, R^{4B} is —CH₂Br. In embodiments, R^{4B} is —CH₂F. In embodiments, R^{4B} is —CH₂I. In embodiments, R^{4B} is —CHCl₂. In embodiments, R^{4B} is —CHBr₂. In embodiments, R^{4B} is —CHF₂. In embodiments, R^{4B} is —CHI₂. In embodiments, R^{4B} is —CN. In embodiments, R^{4B} is —OH. In embodiments, R^{4B} is —NH₂. In embodiments, R^{4B} is —COOH. In embodiments, R^{4B} is —CONH₂. In embodiments, R^{4B} is —NO₂. In embodiments, R^{4B} is —SH. In embodiments, R^{4B} is —SO₃H. In embodiments, R^{4B} is —OSO₃H. In embodiments, R^{4B} is —SO₂NH₂. In embodiments, R^{4B} is —NHNH₂. In embodiments, R^{4B} is —ONH₂. In embodiments, R^{4B} is —NHC(O)NHNH₂. In embodiments, R^{4B} is —NHC(O)NH₂. In embodiments, R^{4B} is —NHSO₂H. In embodiments, R^{4B} is —NHC(O)H. In embodiments, R^{4B} is —NHC(O)OH. In embodiments, R^{4B} is —NHOH. In embodiments, R^{4B} is —OCCl₃. In embodiments, R^{4B} is —OCBr₃. In embodiments, R^{4B} is —OCF₃. In embodiments, R^{4B} is —OCI₃. In embodiments, R^{4B} is —OCH₂Cl. In embodiments, R^{4B} is —OCH₂Br. In embodiments, R^{4B} is —OCH₂F. In embodiments, R^{4B} is —OCH₂I. In embodiments, R^{4B} is —OCHCl₂. In embodiments, R^{4B} is —OCHBr₂. In embodiments, R^{4B} is —OCHF₂. In embodiments, R^{4B} is —OCHI₂. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl. In embodiments, R^{4B} is unsubstituted 2 to 4 membered heteroalkyl. In embodiments, R^{4B} is unsubstituted methoxy. In embodiments, R^{4B} is unsubstituted ethoxy. In embodiments, R^{4B} is unsubstituted propoxy. In embodiments, R^{4B} is unsubstituted n-propoxy. In embodiments, R^{4B} is unsubstituted isopropoxy. In embodiments, R^{4B} is unsubstituted butoxy. In embodiments, R^{4B} is unsubstituted n-butoxy. In embodiments, R^{4B} is unsubstituted isobutoxy. In embodiments, R^{4B} is unsubstituted tert-butoxy.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CCl₃. In embodiments, R⁵ is —CBr₃. In embodiments, R⁵ is —CF₃. In embodiments, R⁵ is —CI₃. In embodiments, R⁵ is —CH₂Cl. In embodiments, R⁵ is —CH₂Br. In embodiments, R⁵ is —CH₂F. In embodiments, R⁵ is —CH₂I. In embodiments, R⁵ is —CHCl₂. In embodiments, R⁵ is —CHBr₂. In embodiments, R⁵ is —CHF₂. In embodiments, R⁵ is —CHI₂. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SO₃H. In embodiments, R⁵ is —OSO₃H. In embodiments, R⁵ is —SO₂NH₂. In embodiments, R⁵ is —NHNH₂. In embodiments, R⁵ is —ONH₂. In embodiments, R⁵ is —NHC(O)NHNH₂. In embodiments, R⁵ is —NHC(O)NH₂. In embodiments, R⁵ is —NHSO₂H. In embodiments, R⁵ is —NHC(O)H. In embodiments, R⁵ is —NHC(O)OH. In embodiments, R⁵ is —NHOH. In embodiments, R⁵ is —OCCl₃. In embodiments, R⁵ is —OCBr₃. In embodiments, R⁵ is —OCF₃. In embodiments, R⁵ is —OCI₃. In embodiments, R⁵ is —OCH₂Cl. In embodiments, R⁵ is —OCH₂Br. In embodiments, R⁵ is —OCH₂F. In embodiments, R⁵ is —OCH₂I. In embodiments, R⁵ is —OCHCl₂. In embodiments, R⁵ is —OCHBr₂. In embodiments, R⁵ is —OCHF₂. In embodiments, R⁵ is —OCHI₂. In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted 2 to 4 membered heteroalkyl. In embodiments, R⁵ is unsubstituted methoxy. In embodiments, R⁵ is unsubstituted ethoxy. In embodiments, R⁵ is unsubstituted propoxy. In embodiments, R⁵ is unsubstituted n-propoxy. In embodiments, R⁵ is unsubstituted isopropoxy. In embodiments, R⁵ is unsubstituted butoxy. In embodiments, R⁵ is unsubstituted n-butoxy. In embodiments, R⁵ is unsubstituted isobutoxy. In embodiments, R⁵ is unsubstituted tert-butoxy.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I. In embodiments, R⁶ is —CCl₃. In embodiments, R⁶ is —CBr₃. In embodiments, R⁶ is —CF₃. In embodiments, R⁶ is —CI₃. In embodiments, R⁶ is —CH₂C₁. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃H. In embodiments, R⁶ is —OSO₃H. In embodiments, R⁶ is —SO₂NH₂. In embodiments, R⁶ is —NHNH₂. In embodiments, R⁶ is —ONH₂. In embodiments, R⁶ is —NHC(O)NHNH₂. In embodiments, R⁶ is —NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCBr₃. In embodiments, R⁶ is —OCF₃. In embodiments, R⁶ is —OCI₃. In embodiments, R⁶ is —OCH₂Cl. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted 2 to 4 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy. In embodiments, R⁶ is unsubstituted n-butoxy. In embodiments, R⁶ is unsubstituted isobutoxy. In embodiments, R⁶ is unsubstituted tert-butoxy.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —I. In embodiments, R⁷ is —CCl₃. In embodiments, R⁷ is —CBr₃. In embodiments, R⁷ is —CF₃. In embodiments, R⁷ is —CI₃. In embodiments, R⁷ is —CH₂C₁. In embodiments, R⁷ is —CH₂Br. In embodiments, R⁷ is —CH₂F. In embodiments, R⁷ is —CH₂I. In embodiments, R⁷ is —CHCl₂. In embodiments, R⁷ is —CHBr₂. In embodiments, R⁷ is —CHF₂. In embodiments, R⁷ is —CHI₂. In embodiments, R⁷ is —CN. In embodiments, R⁷ is —OH. In embodiments, R⁷ is —NH₂. In embodiments, R⁷ is —COOH. In embodiments, R⁷ is —CONH₂. In embodiments, R⁷ is —NO₂. In embodiments, R⁷ is —SH. In embodiments, R⁷ is —SO₃H. In embodiments, R⁷ is —OSO₃H. In embodiments, R⁷ is —SO₂NH₂. In embodiments, R⁷ is —NHNH₂. In embodiments, R⁷ is —ONH₂. In embodiments, R⁷ is —NHC(O)NHNH₂. In embodiments, R⁷ is —NHC(O)NH₂. In embodiments, R⁷ is —NHSO₂H. In embodiments, R⁷ is —NHC(O)H. In embodiments, R⁷ is —NHC(O)OH. In embodiments, R⁷ is —NHOH. In embodiments, R⁷ is —OCCl₃. In embodiments, R⁷ is —OCBr₃. In embodiments, R⁷ is —OCF₃. In embodiments, R⁷ is —OCI₃. In embodiments, R⁷ is —OCH₂Cl. In embodiments, R⁷ is —OCH₂Br. In embodiments, R⁷ is —OCH₂F. In embodiments, R⁷ is —OCH₂I. In embodiments, R⁷ is —OCHCl₂. In embodiments, R⁷ is —OCHBr₂. In embodiments, R⁷ is —OCHF₂. In embodiments, R⁷ is —OCHI₂. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted 2 to 4 membered heteroalkyl. In embodiments, R⁷ is unsubstituted methoxy. In embodiments, R⁷ is unsubstituted ethoxy. In embodiments, R⁷ is unsubstituted propoxy. In embodiments, R⁷ is unsubstituted n-propoxy. In embodiments, R⁷ is unsubstituted isopropoxy. In embodiments, R⁷ is unsubstituted butoxy. In embodiments, R⁷ is unsubstituted n-butoxy. In embodiments, R⁷ is unsubstituted isobutoxy. In embodiments, R⁷ is unsubstituted tert-butoxy.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen or unsubstituted C₁-C₄ alkyl. In embodiments, R′ is hydrogen. In embodiments, R⁸ is unsubstituted C₁-C₄ alkyl. In embodiments, R⁸ is unsubstituted methyl. In embodiments, R⁸ is unsubstituted ethyl. In embodiments, R′ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyle (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁹ is independently halogen. In embodiments, R⁹ is independently —F. In embodiments, R⁹ is independently —Cl. In embodiments, R⁹ is independently —Br. In embodiments, R⁹ is independently —I. In embodiments, R⁹ is independently —CCl₃. In embodiments, R⁹ is independently —CBr₃. In embodiments, R⁹ is independently —CF₃. In embodiments, R⁹ is independently —CI₃. In embodiments, R⁹ is independently —CH₂C₁. In embodiments, R⁹ is independently —CH₂Br. In embodiments, R⁹ is independently —CH₂F. In embodiments, R⁹ is independently —CH₂I. In embodiments, R⁹ is independently —CHCl₂. In embodiments, R⁹ is independently —CHBr₂. In embodiments, R⁹ is independently —CHF₂. In embodiments, R⁹ is independently —CHI₂. In embodiments, R⁹ is independently —CN. In embodiments, R⁹ is independently —OR^{9D}. In embodiments, R⁹ is independently —OCH₃. In embodiments, R⁹ is independently —OH. In embodiments, R⁹ is independently —NH₂. In embodiments, R⁹ is independently —COOH. In embodiments, R⁹ is independently —CONH₂. In embodiments, R⁹ is independently —NO₂. In embodiments, R⁹ is independently —SH. In embodiments, R⁹ is independently —SO₃H. In embodiments, R⁹ is independently —OSO₃H. In embodiments, R⁹ is independently —SO₂NH₂. In embodiments, R⁹ is independently —NHNH₂. In embodiments, R⁹ is independently —ONH₂. In embodiments, R⁹ is independently —NHC(O)NHNH₂. In embodiments, R⁹ is independently —NHC(O)NH₂. In embodiments, R⁹ is independently —NHSO₂H. In embodiments, R⁹ is independently —NHC(O)H. In embodiments, R⁹ is independently —NHC(O)OH. In embodiments, R⁹ is independently —NHOH. In embodiments, R⁹ is independently —OCCl₃. In embodiments, R⁹ is independently —OCBr₃. In embodiments, R⁹ is independently —OCF₃. In embodiments, R⁹ is independently —OCI₃. In embodiments, R⁹ is independently —OCH₂Cl. In embodiments, R⁹ is independently —OCH₂Br. In embodiments, R⁹ is independently —OCH₂F. In embodiments, R⁹ is independently —OCH₂I. In embodiments, R⁹ is independently —OCHCl₂. In embodiments, R⁹ is independently —OCHBr₂. In embodiments, R⁹ is independently —OCHF₂. In embodiments, R⁹ is independently —OCHI₂. In embodiments, R⁹ is independently unsubstituted C₁-C₄ alkyl. In embodiments, R⁹ is independently unsubstituted methyl. In embodiments, R⁹ is independently unsubstituted ethyl. In embodiments, R⁹ is independently unsubstituted propyl. In embodiments, R⁹ is independently unsubstituted n-propyl. In embodiments, R⁹ is independently unsubstituted isopropyl. In embodiments, R⁹ is independently unsubstituted butyl. In embodiments, R⁹ is independently unsubstituted n-butyl. In embodiments, R⁹ is independently unsubstituted isobutyl. In embodiments, R⁹ is independently unsubstituted tert-butyl. In embodiments, R⁹ is independently unsubstituted 2 to 4 membered heteroalkyl. In embodiments, R⁹ is independently unsubstituted methoxy. In embodiments, R⁹ is independently unsubstituted ethoxy. In embodiments, R⁹ is independently unsubstituted propoxy. In embodiments, R⁹ is independently unsubstituted n-propoxy. In embodiments, R⁹ is independently unsubstituted isopropoxy. In embodiments, R⁹ is independently unsubstituted butoxy. In embodiments, R⁹ is independently unsubstituted n-butoxy. In embodiments, R⁹ is independently unsubstituted isobutoxy. In embodiments, R⁹ is independently unsubstituted tert-butoxy.

In embodiments, a substituted R^{9A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, a substituted R^{9B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9B} is substituted with a plurality of groups selected from substituent groups, size-limited substituent groups, and lower substituent groups; each substituent group, size-limited substituent group, and/or lower substituent group may optionally be different. In embodiments, when R^{9B} is substituted, it is substituted with at least one substituent group. In embodiments, when R^{9B} is substituted, it is substituted with at least one size-limited substituent group. In embodiments, when R^{9B} is substituted, it is substituted with at least one lower substituent group.

In embodiments, a substituted ring formed when R^{9A} and R^{9B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{9A} and R⁹ substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{9A} and R^{9B} substituents bonded to the same nitrogen atom are joined is substituted, it is substituted with at least one substituent group. In embodiments, when the substituted ring formed when R^{9A} and R^{9B} substituents bonded to the same nitrogen atom are joined is substituted, it is substituted with at least one size-limited substituent group. In embodiments, when the substituted ring formed when R^{9A} and R^{9B} substituents bonded to the same nitrogen atom are joined is substituted, it is substituted with at least one lower substituent group.

In embodiments, a substituted R^{9C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9C} is substituted with a plurality of groups selected from substituent groups, size-limited substituent groups, and lower substituent groups; each substituent group, size-limited substituent group, and/or lower substituent group may optionally be different. In embodiments, when R^{9C} is substituted, it is substituted with at least one substituent group. In embodiments, when R^{9C} is substituted, it is substituted with at least one size-limited substituent group. In embodiments, when R^{9C} is substituted, it is substituted with at least one lower substituent group.

In embodiments, a substituted R^{9D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9D} is substituted with a plurality of groups selected from substituent groups, size-limited substituent groups, and lower substituent groups; each substituent group, size-limited substituent group, and/or lower substituent group may optionally be different. In embodiments, when R^{9D} is substituted, it is substituted with at least one substituent group. In embodiments, when R^{9D} is substituted, it is substituted with at least one size-limited substituent group. In embodiments, when R^{9D} is substituted, it is substituted with at least one lower substituent group.

In embodiments, R^{9A} is independently hydrogen or unsubstituted C₁-C₄ alkyl. In embodiments, R^{9A} is independently hydrogen. In embodiments, R^{9A} is independently unsubstituted C₁-C₄ alkyl. In embodiments, R^{9A} is independently unsubstituted methyl. In embodiments, R^{9A} is independently unsubstituted ethyl. In embodiments, R^{9A} is independently unsubstituted propyl. In embodiments, R^{9A} is independently unsubstituted n-propyl. In embodiments, R^{9A} is independently unsubstituted isopropyl. In embodiments, R^{9A} is independently unsubstituted butyl. In embodiments, R^{9A} is independently unsubstituted n-butyl. In embodiments, R^{9A} is independently unsubstituted isobutyl. In embodiments, R^{9A} is independently unsubstituted tert-butyl.

In embodiments, R^{9B} is independently hydrogen or unsubstituted C₁-C₄ alkyl. In embodiments, R^{9B} is independently hydrogen. In embodiments, R^{9B} is independently unsubstituted C₁-C₄ alkyl. In embodiments, R^{9B} is independently unsubstituted methyl. In embodiments, R^{9B} is independently unsubstituted ethyl. In embodiments, R^{9B} is independently unsubstituted propyl. In embodiments, R^{9B} is independently unsubstituted n-propyl. In embodiments, R^{9B} is independently unsubstituted isopropyl. In embodiments, R^{9B} is independently unsubstituted butyl. In embodiments, R^{9B} is independently unsubstituted n-butyl. In embodiments, R^{9B} is independently unsubstituted isobutyl. In embodiments, R^{9B} is independently unsubstituted tert-butyl.

In embodiments, R^{9C} is independently hydrogen or unsubstituted C₁-C₄ alkyl. In embodiments, R^{9C} is independently hydrogen. In embodiments, R^{9C} is independently unsubstituted C₁-C₄ alkyl. In embodiments, R^{9C} is independently unsubstituted methyl. In embodiments, R^{9C} is independently unsubstituted ethyl. In embodiments, R^{9C} is independently unsubstituted propyl. In embodiments, R^{9C} is independently unsubstituted n-propyl. In embodiments, R^{9C} is independently unsubstituted isopropyl. In embodiments, R^{9C} is independently unsubstituted butyl. In embodiments, R^{9C} is independently unsubstituted n-butyl. In embodiments, R^{9C} is independently unsubstituted isobutyl. In embodiments, R^{9C} is independently unsubstituted tert-butyl.

In embodiments, R^{9D} is independently hydrogen or unsubstituted C₁-C₄ alkyl. In embodiments, R^{9D} is independently hydrogen. In embodiments, R^{9D} is independently unsubstituted C₁-C₄ alkyl. In embodiments, R^{9D} is independently unsubstituted methyl. In embodiments, R^{9D} is independently unsubstituted ethyl. In embodiments, R^{9D} is independently unsubstituted propyl. In embodiments, R^{9D} is independently unsubstituted n-propyl. In embodiments, R^{9D} is independently unsubstituted isopropyl. In embodiments, R^{9D} is independently unsubstituted butyl. In embodiments, R^{9D} is independently unsubstituted n-butyl. In embodiments, R^{9D} is independently unsubstituted isobutyl. In embodiments, R^{9D} is independently unsubstituted tert-butyl.

In embodiments, z9 is 0. In embodiments, z9 is 1. In embodiments, z9 is 2. In embodiments, z9 is 3. In embodiments, z9 is 4.

In embodiments, a substituted group formed when R^{3A} and R^{3B} substituents are combined (e.g., substituted alkenyl, substituted cycloalkyl, and/or substituted heterocycloalkyl) is substituted with at least one substituent group, size-limited substituent group, or lower substituent group; wherein if the substituted group formed when R^{3A} and R^{3B} substituents are combined is substituted with a plurality of groups selected from substituent groups, size-limited substituent groups, and lower substituent groups; each substituent group, size-limited substituent group, and/or lower substituent group may optionally be different. In embodiments, when the substituted group formed when R^{3A} and R^{3B} substituents are combined is substituted, it is substituted with at least one substituent group. In embodiments, when the substituted group formed when R^{3A} and R^{3B} substituents are combined is substituted, it is substituted with at least one size-limited substituent group. In embodiments, when the substituted group formed when R^{3A} and R^{3B} substituents are combined is substituted, it is substituted with at least one lower substituent group.

In embodiments, a substituted group formed when R^{4A} and R^{4B} substituents are combined (e.g., substituted alkenyl, substituted cycloalkyl, and/or substituted heterocycloalkyl) is substituted with at least one substituent group, size-limited substituent group, or lower substituent group; wherein if the substituted group formed when R^{4A} and R^{4B} substituents are combined is substituted with a plurality of groups selected from substituent groups, size-limited substituent groups, and lower substituent groups; each substituent group, size-limited substituent group, and/or lower substituent group may optionally be different. In embodiments, when the substituted group formed when R^{4A} and R^{4B} substituents are combined is substituted, it is substituted with at least one substituent group. In embodiments, when the substituted group formed when R^{4A} and R^{4B} substituents are combined is substituted, it is substituted with at least one size-limited substituent group. In embodiments, when the substituted group formed when R^{4A} and R^{4B} substituents are combined is substituted, it is substituted with at least one lower substituent group.

In embodiments, a substituted ring formed when R² and R^{3A} substituents, together with the carbon atom to which they are attached, are joined (e.g., substituted cycloalkyl and/or substituted heterocycloalkyl) is substituted with at least one substituent group, size-limited substituent group, or lower substituent group; wherein if the substituted ring formed when R² and R^{3A} substituents, together with the carbon atom to which they are attached, are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R^{3A} substituents, together with the carbon atom to which they are attached, are joined is substituted, it is substituted with at least one substituent group. In embodiments, when the substituted ring formed when R² and R^{3A} substituents, together with the carbon atom to which they are attached, are joined is substituted, it is substituted with at least one size-limited substituent group. In embodiments, when the substituted ring formed when R² and R^{3A} substituents, together with the carbon atom to which they are attached, are joined is substituted, it is substituted with at least one lower substituent group.

In embodiments, a substituted ring formed when R^{3A} and R^{4A} substituents, together with the carbon atom to which they are attached, are joined (e.g., substituted cycloalkyl and/or substituted heterocycloalkyl) is substituted with at least one substituent group, size-limited substituent group, or lower substituent group; wherein if the substituted ring formed when R^{3A} and R^{4A} substituents, together with the carbon atom to which they are attached, are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{4A} substituents, together with the carbon atom to which they are attached, are joined is substituted, it is substituted with at least one substituent group. In embodiments, when the substituted ring formed when R^{3A} and R^{4A} substituents, together with the carbon atom to which they are attached, are joined is substituted, it is substituted with at least one size-limited substituent group. In embodiments, when the substituted ring formed when R^{3A} and R^{4A} substituents, together with the carbon atom to which they are attached, are joined is substituted, it is substituted with at least one lower substituent group.

In embodiments, a substituted ring formed when R^{3B} and R^{4B} substituents, together with the carbon atom to which they are attached, are joined (e.g., substituted cycloalkyl and/or substituted heterocycloalkyl) is substituted with at least one substituent group, size-limited substituent group, or lower substituent group; wherein if the substituted ring formed when R^{3B} and R^{4B} substituents, together with the carbon atom to which they are attached, are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3B} and R^{4B} substituents, together with the carbon atom to which they are attached, are joined is substituted, it is substituted with at least one substituent group. In embodiments, when the substituted ring formed when R^{3B} and R^{4B} substituents, together with the carbon atom to which they are attached, are joined is substituted, it is substituted with at least one size-limited substituent group. In embodiments, when the substituted ring formed when R^{3B} and R^{4B} substituents, together with the carbon atom to which they are attached, are joined is substituted, it is substituted with at least one lower substituent group.

In embodiments, a substituted ring formed when R^{4A} and R⁵ substituents, together with the carbon atom to which they are attached, are joined (e.g., substituted cycloalkyl and/or substituted heterocycloalkyl) is substituted with at least one substituent group, size-limited substituent group, or lower substituent group; wherein if the substituted ring formed when R^{4A} and R⁵ substituents, together with the carbon atom to which they are attached, are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R⁵ substituents, together with the carbon atom to which they are attached, are joined is substituted, it is substituted with at least one substituent group. In embodiments, when the substituted ring formed when R^{4A} and R⁵ substituents, together with the carbon atom to which they are attached, are joined is substituted, it is substituted with at least one size-limited substituent group. In embodiments, when the substituted ring formed when R^{4A} and R⁵ substituents, together with the carbon atom to which they are attached, are joined is substituted, it is substituted with at least one lower substituent group.

In embodiments, the compound has the formula:

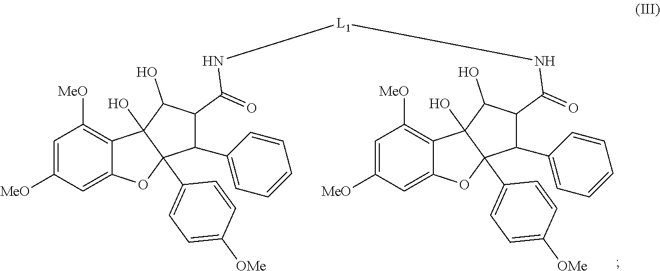

wherein L¹ is the covalent linker.

In embodiments, the compound has the formula:

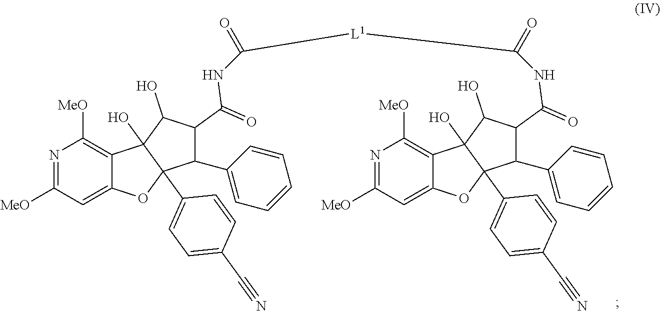

wherein L¹ is the covalent linker.

In embodiments, L¹ is the covalent linker. In embodiments, L¹ is -L¹⁰¹-L¹⁰²-L¹⁰³-.

L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹—, —C(NH)NR¹⁰¹—, —S(O)₂—, —NR¹⁰¹(O)₂—, —S(O)₂NR¹⁰¹—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(N¹⁰²)NR¹⁰²—, —S(O)₂—, —NR¹⁰²S(O)₂—, —S(O)₂NR¹⁰²—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰³ is a bond, —C(O)—, —C(O)O—, —OC(O)—, —O—, —S—, —NR⁰³—, —C(O)NR⁰³—, —NR¹⁰³C(O)—, —NR¹⁰³C(O)O—, —OC(O)NR¹⁰³—, —NR¹⁰³C(O)NR¹⁰³—, —NR⁰³C(NH)NR¹⁰³—, —S(O)₂—, —NR¹⁰³S(O)₂—, —S(O)₂NR¹⁰³—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

Each R¹⁰¹, R¹⁰², and R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰¹ is a bond. In embodiments, L¹⁰¹ is —C(O)—. In embodiments, L¹⁰¹ is —C(O)O—. In embodiments, L¹⁰¹ is —OC(O)—. In embodiments, L¹⁰¹ is —O—. In embodiments, L¹⁰¹ is —S—. In embodiments, L¹⁰¹ is —NR¹⁰¹—. In embodiments, L¹⁰¹ is —NH—. In embodiments, L¹⁰¹ is —C(O)NR¹⁰¹—. In embodiments, L¹⁰¹ is —C(O)NH—. In embodiments, L¹⁰¹ is —NR¹⁰¹C(O)—. In embodiments, L¹⁰¹ is —NHC(O)—. In embodiments, L¹⁰¹ is —NR¹⁰¹C(O)O—. In embodiments, L¹⁰¹ is —NHC(O)O—. In embodiments, L¹⁰¹ is —OC(O)NR¹⁰¹—. In embodiments, L¹⁰¹ is —OC(O)NH—. In embodiments, L¹⁰¹ is —NR¹⁰¹C(O)NR⁰¹—. In embodiments, L¹⁰¹ is —NHC(O)NH—. In embodiments, L¹⁰¹ is —NR¹⁰¹C(NH)NR¹⁰¹—. In embodiments, L¹⁰¹ is —NHC(NH)NH—. In embodiments, L¹⁰¹ is —S(O)₂—. In embodiments, L¹⁰¹ is —NR¹⁰¹S(O)₂—. In embodiments, L¹⁰¹ is —NHS(O)₂—. In embodiments, L¹⁰¹ is —S(O)₂NR¹⁰¹—. In embodiments, L¹⁰¹ is —S(O)₂NH—. In embodiments, L¹⁰¹ is unsubstituted C₁-C₆ alkylene. In embodiments, L¹⁰¹ is unsubstituted methylene. In embodiments, L¹⁰¹ is unsubstituted ethylene. In embodiments, L¹⁰¹ is unsubstituted propylene. In embodiments, L¹⁰¹ is unsubstituted n-propylene. In embodiments, L¹⁰¹ is unsubstituted isopropylene. In embodiments, L¹⁰¹ is unsubstituted butylene. In embodiments, L¹⁰¹ is unsubstituted n-butylene. In embodiments, L¹⁰¹ is unsubstituted isobutylene. In embodiments, L¹⁰¹ is unsubstituted tert-butylene. In embodiments, L¹⁰¹ is unsubstituted pentylene. In embodiments, L¹⁰¹ is unsubstituted hexylene. In embodiments, L¹⁰¹ is unsubstituted 2 to 30 membered heteroalkylene. In embodiments, L¹⁰¹ is

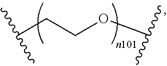

wherein n101 is an integer from 1 to 50. In embodiments, L¹⁰¹ is

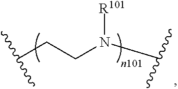

wherein R¹⁰¹ is independently hydrogen or unsubstituted C₁-C₄ alkyl; and n101 is an integer from 1 to 50. In embodiments, L¹⁰¹ is

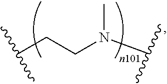

wherein n101 is an integer from 1 to 50. In embodiments, L¹⁰¹ is

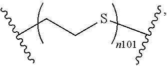

wherein n101 is an integer from 1 to 50. In embodiments, L¹⁰¹ is

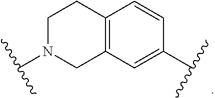 [SMILES: *c1ccc2c(c1)CN(*)CC2]

In embodiments, L¹⁰¹ is unsubstituted C₂-C₄₀ alkylene.

In embodiments, n101 is an integer from 1 to 20. In embodiments, n101 is an integer from 1 to 15. In embodiments, n101 is an integer from 1 to 10. In embodiments, n101 is an integer from 1 to 5. In embodiments, n101 is 1. In embodiments, n101 is 2. In embodiments, n101 is 3. In embodiments, n101 is 4. In embodiments, n101 is 5. In embodiments, n101 is 6. In embodiments, n101 is 7. In embodiments, n101 is 8. In embodiments, n101 is 9. In embodiments, n101 is 10. In embodiments, n101 is 11. In embodiments, n101 is 12. In embodiments, n101 is 13. In embodiments, n101 is 14. In embodiments, n101 is 15. In embodiments, n101 is 16. In embodiments, n101 is 17. In embodiments, n101 is 18. In embodiments, n101 is 19. In embodiments, n101 is 20.

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is independently hydrogen or unsubstituted C₁-C₄ alkyl. In embodiments, R¹⁰¹ is independently hydrogen. In embodiments, R¹⁰¹ is independently unsubstituted C₁-C₄ alkyl. In embodiments, R¹⁰¹ is independently unsubstituted methyl. In embodiments, R¹⁰¹ is independently unsubstituted ethyl. In embodiments, R¹⁰¹ is independently unsubstituted propyl. In embodiments, R¹⁰¹ is independently unsubstituted n-propyl. In embodiments, R¹⁰¹ is independently unsubstituted isopropyl. In embodiments, R¹⁰¹ is independently unsubstituted butyl. In embodiments, R¹⁰¹ is independently unsubstituted n-butyl. In embodiments, R¹⁰¹ is independently unsubstituted isobutyl. In embodiments, R¹⁰¹ is independently unsubstituted tert-butyl.

In embodiments, a substituted L¹⁰² (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰² is a bond. In embodiments, L¹⁰² is —C(O)—. In embodiments, L¹⁰² is —C(O)O—. In embodiments, L¹⁰² is —OC(O)—. In embodiments, L¹⁰² is —O—. In embodiments, L¹⁰² is —S—. In embodiments, L¹⁰² is —NR¹⁰²—. In embodiments, L¹⁰² is —NH—. In embodiments, L¹⁰² is —C(O)NR¹⁰²—. In embodiments, L¹⁰² is —C(O)NH—. In embodiments, L¹⁰² is —NR¹⁰²C(O)—. In embodiments, L¹⁰² is —NHC(O)—. In embodiments, L¹⁰² is —NR¹⁰²C(O)O—. In embodiments, L¹⁰² is —NHC(O)O—. In embodiments, L¹⁰² is —OC(O)NR¹⁰²—. In embodiments, L¹⁰² is —OC(O)NH—. In embodiments, L¹⁰² is —NR¹⁰²C(O)NR¹⁰²—. In embodiments, L¹⁰² is —NHC(O)NH—. In embodiments, L¹⁰² is —NR¹⁰²C(NH)NR¹⁰²—. In embodiments, L¹⁰² is —NHC(NH)NH—. In embodiments, L¹⁰² is —S(O)₂—. In embodiments, L¹⁰² is —NR¹⁰²S(O)₂—. In embodiments, L¹⁰² is —NHS(O)₂—. In embodiments, L¹⁰² is —S(O)₂NR¹⁰²—. In embodiments, L¹⁰² is —S(O)₂NH—. In embodiments, L¹⁰² is unsubstituted C₁-C₆ alkylene. In embodiments, L¹⁰² is unsubstituted methylene. In embodiments, L¹⁰² is unsubstituted ethylene. In embodiments, L¹⁰² is unsubstituted propylene. In embodiments, L¹⁰² is unsubstituted n-propylene. In embodiments, L¹⁰² is unsubstituted isopropylene. In embodiments, L¹⁰² is unsubstituted butylene. In embodiments, L¹⁰² is unsubstituted n-butylene. In embodiments, L¹⁰² is unsubstituted isobutylene. In embodiments, L¹⁰² is unsubstituted tert-butylene. In embodiments, L¹⁰² is unsubstituted pentylene. In embodiments, L¹⁰² is unsubstituted hexylene. In embodiments, L¹⁰² is unsubstituted 2 to 30 membered heteroalkylene. In embodiments, L¹⁰² is

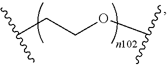

wherein n102 is an integer from 1 to 50. In embodiments, L¹⁰² is

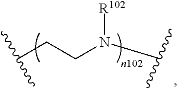

wherein R¹⁰² is independently hydrogen or unsubstituted C₁-C₄ alkyl; and n102 is an integer from 1 to 50. In embodiments, L¹⁰² is

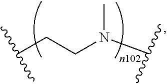

wherein n102 is an integer from 1 to 50. In embodiments, L¹⁰² is

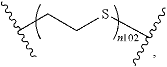

wherein n102 is an integer from 1 to 50. In embodiments, L¹⁰² is substituted or unsubstituted 2 to 100 membered heteroalkylene. In embodiments, L¹⁰² is

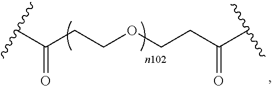

wherein n102 is an integer from 1 to 50. In embodiments, L¹⁰² is unsubstituted C₂-C₄₀ alkylene.

In embodiments, n102 is an integer from 1 to 20. In embodiments, n102 is an integer from 1 to 15. In embodiments, n102 is an integer from 1 to 10. In embodiments, n102 is an integer from 1 to 5. In embodiments, n102 is 1. In embodiments, n102 is 2. In embodiments, n102 is 3. In embodiments, n102 is 4. In embodiments, n102 is 5. In embodiments, n102 is 6. In embodiments, n102 is 7. In embodiments, n102 is 8. In embodiments, n102 is 9. In embodiments, n102 is 10. In embodiments, n102 is 11. In embodiments, n102 is 12. In embodiments, n102 is 13. In embodiments, n102 is 14. In embodiments, n102 is 15. In embodiments, n102 is 16. In embodiments, n102 is 17. In embodiments, n102 is 18. In embodiments, n102 is 19. In embodiments, n102 is 20.

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently hydrogen or unsubstituted C₁-C₄ alkyl. In embodiments, R¹⁰² is independently hydrogen. In embodiments, R¹⁰² is independently unsubstituted C₁-C₄ alkyl. In embodiments, R¹⁰² is independently unsubstituted methyl. In embodiments, R¹⁰² is independently unsubstituted ethyl. In embodiments, R¹⁰² is independently unsubstituted propyl. In embodiments, R¹⁰² is independently unsubstituted n-propyl. In embodiments, R¹⁰² is independently unsubstituted isopropyl. In embodiments, R¹⁰² is independently unsubstituted butyl. In embodiments, R¹⁰² is independently unsubstituted n-butyl. In embodiments, R¹⁰² is independently unsubstituted isobutyl. In embodiments, R¹⁰² is independently unsubstituted tert-butyl.

In embodiments, a substituted L¹⁰³ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰³ is a bond. In embodiments, L¹⁰³ is —C(O)—. In embodiments, L¹⁰³ is —C(O)O—. In embodiments, L¹⁰³ is —OC(O)—. In embodiments, L¹⁰³ is —O—. In embodiments, L¹⁰³ is —S—. In embodiments, L¹⁰³ is —NR¹⁰³—. In embodiments, L¹⁰³ is —NH—. In embodiments, L¹⁰³ is —C(O)NR¹⁰³—. In embodiments, L¹⁰³ is —C(O)NH—. In embodiments, L¹⁰³ is —NR¹⁰³C(O)—. In embodiments, L¹⁰³ is —NHC(O)—. In embodiments, L¹⁰³ is —NR¹⁰³C(O)O—. In embodiments, L¹⁰³ is —NHC(O)O—. In embodiments, L¹⁰³ is —OC(O)NR¹⁰³—. In embodiments, L¹⁰³ is —OC(O)NH—. In embodiments, L¹⁰³ is —NR¹⁰³C(O)NR¹⁰³—. In embodiments, L¹⁰³ is —NHC(O)NH—. In embodiments, L¹⁰³ is —NR¹⁰³C(NH)NR¹⁰³—. In embodiments, L¹⁰³ is —NHC(NH)NH—. In embodiments, L¹⁰³ is —S(O)₂—. In embodiments, L¹⁰³ is —NR¹⁰³S(O)₂—. In embodiments, L¹⁰³ is —NHS(O)₂—. In embodiments, L¹⁰³ is —S(O)₂NR¹⁰³—. In embodiments, L¹⁰³ is —S(O)₂NH—. In embodiments, L¹⁰³ is unsubstituted C₁-C₆ alkylene. In embodiments, L¹⁰³ is unsubstituted methylene. In embodiments, L¹⁰³ is unsubstituted ethylene. In embodiments, L¹⁰³ is unsubstituted propylene. In embodiments, L¹⁰³ is unsubstituted n-propylene. In embodiments, L¹⁰³ is unsubstituted isopropylene. In embodiments, L¹⁰³ is unsubstituted butylene. In embodiments, L¹⁰³ is unsubstituted n-butylene. In embodiments, L¹⁰³ is unsubstituted isobutylene. In embodiments, L¹⁰³ is unsubstituted tert-butylene. In embodiments, L¹⁰³ is unsubstituted pentylene. In embodiments, L¹⁰³ is unsubstituted hexylene. In embodiments, L¹⁰³ is unsubstituted 2 to 30 membered heteroalkylene. In embodiments, L¹⁰³ is

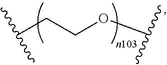

wherein n103 is an integer from 1 to 50. In embodiments, L¹⁰³ is

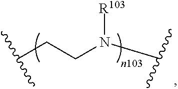

wherein R¹⁰³ is independently hydrogen or unsubstituted C₁-C₄ alkyl; and n103 is an integer from 1 to 50. In embodiments, L¹⁰³ is

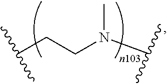

wherein n103 is an integer from 1 to 50. In embodiments, L¹⁰³ is

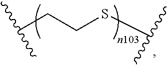

wherein n103 is an integer from 1 to 50. In embodiments, L¹⁰³ is

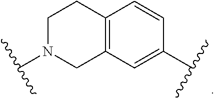 [SMILES: *c1ccc2c(c1)CN(*)CC2]

In embodiments, L¹⁰³ is unsubstituted C₂-C₄₀ alkylene.

In embodiments, n103 is an integer from 1 to 20. In embodiments, n103 is an integer from 1 to 15. In embodiments, n103 is an integer from 1 to 10. In embodiments, n103 is an integer from 1 to 5. In embodiments, n103 is 1. In embodiments, n103 is 2. In embodiments, n103 is 3. In embodiments, n103 is 4. In embodiments, n103 is 5. In embodiments, n103 is 6. In embodiments, n103 is 7. In embodiments, n103 is 8. In embodiments, n103 is 9. In embodiments, n103 is 10. In embodiments, n103 is 11. In embodiments, n103 is 12. In embodiments, n103 is 13. In embodiments, n103 is 14. In embodiments, n103 is 15. In embodiments, n103 is 16. In embodiments, n103 is 17. In embodiments, n103 is 18. In embodiments, n103 is 19. In embodiments, n103 is 20.

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is independently hydrogen or unsubstituted C₁-C₄ alkyl. In embodiments, R¹⁰³ is independently hydrogen. In embodiments, R¹⁰³ is independently unsubstituted C₁-C₄ alkyl. In embodiments, R¹⁰³ is independently unsubstituted methyl. In embodiments, R¹⁰³ is independently unsubstituted ethyl. In embodiments, R¹⁰³ is independently unsubstituted propyl. In embodiments, R¹⁰³ is independently unsubstituted n-propyl. In embodiments, R¹⁰³ is independently unsubstituted isopropyl. In embodiments, R¹⁰³ is independently unsubstituted butyl. In embodiments, R¹⁰³ is independently unsubstituted n-butyl. In embodiments, R¹⁰³ is independently unsubstituted isobutyl. In embodiments, R¹⁰³ is independently unsubstituted tert-butyl.

In embodiments, L¹ is

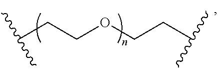

wherein n is an integer from 1 to 50. In embodiments, L¹ is

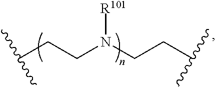

wherein R¹⁰¹ is independently hydrogen or unsubstituted C₁-C₄ alkyl; and n is an integer from 1 to 50. In embodiments, L¹ is

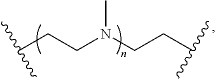

wherein n is an integer from 1 to 50. In embodiments, L¹ is

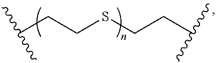

wherein n is an integer from 1 to 50. In embodiments, L¹ is

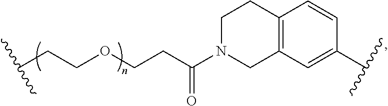

wherein n is an integer from 1 to 50. In embodiments, L¹ is

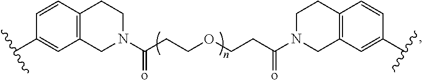

wherein n is an integer from 1 to 50. In embodiments, L¹ is unsubstituted C₂-C₄₀ alkylene.

In embodiments, n is an integer from 1 to 20. In embodiments, n is an integer from 1 to 15. In embodiments, n is an integer from 1 to 10. In embodiments, n is an integer from 1 to 5. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20.

In embodiments, L¹ is a linker as described in Fouda, A. E. et al., Angew. Chem. Int. Ed. 2015, 54, 9618-9621, which is herein incorporated by reference in its entirety for all purposes. In embodiments, L¹ is a linker as described in Troup, R. I. et al., Explor. Target Antitumor Ther. 2020, 1, 273-312, which is herein incorporated by reference in its entirety for all purposes.

In embodiments, the compound has the formula:

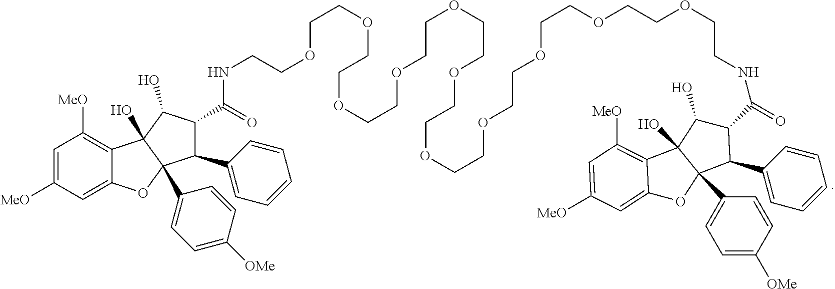 [SMILES: COc1ccc([C@@]23Oc4cc(OC)cc(OC)c4[C@]2(O)[C@H](O)[C@H](C(=O)NCCOCCOCCOCCOCCOCCOCCOCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

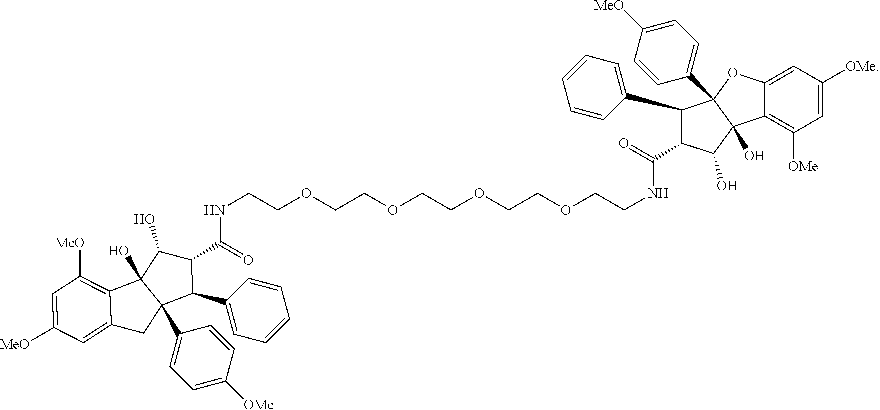 [SMILES: COc1ccc([C@@]23Cc4cc(OC)cc(OC)c4[C@]2(O)[C@H](O)[C@H](C(=O)NCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

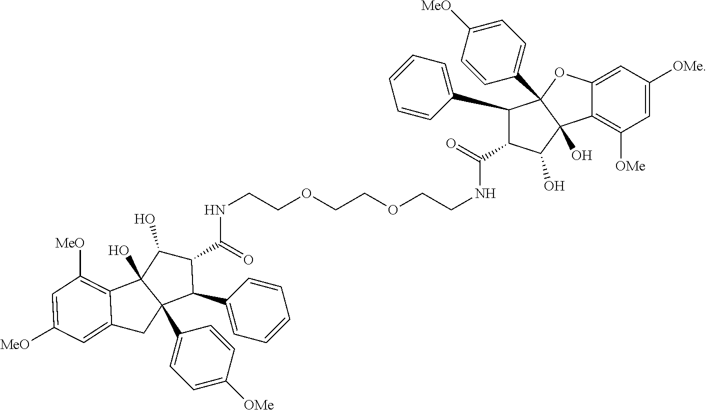 [SMILES: COc1ccc([C@@]23Cc4cc(OC)cc(OC)c4[C@]2(O)[C@H](O)[C@H](C(=O)NCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

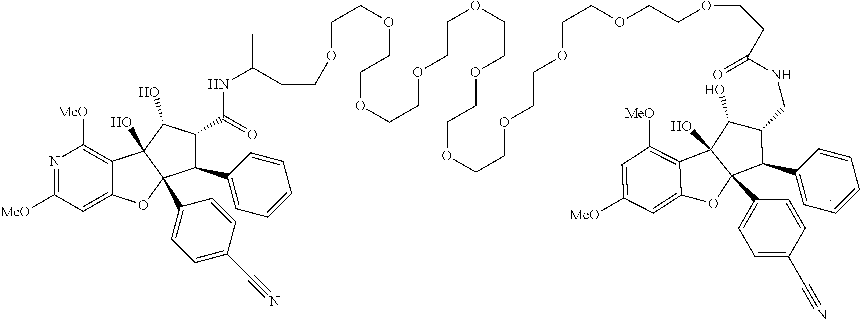 [SMILES: COc1cc(OC)c2c(c1)O[C@@]1(c3ccc(C#N)cc3)[C@H](c3ccccc3)[C@@H](CNC(=O)CCOCCOCCOCCOCCOCCOCCOCCOCCOCCOCCOCCC(C)NC(=O)[C@H]3[C@@H](O)[C@@]4(O)c5c(cc(OC)nc5OC)O[C@@]4(c4ccc(C#N)cc4)[C@@H]3c3ccccc3)[C@@H](O)[C@@]21O]

In embodiments, the compound has the formula:

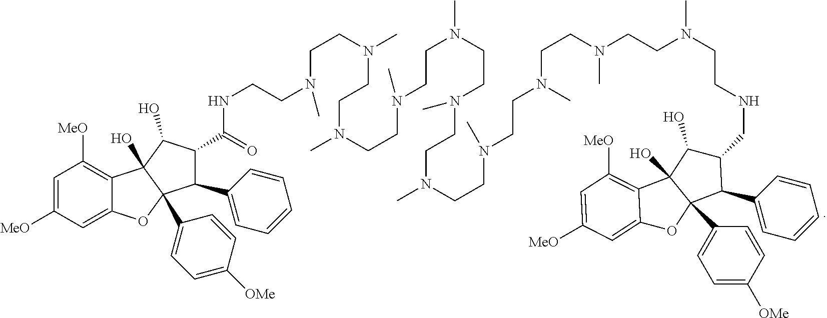 [SMILES: COc1ccc([C@@]23Oc4cc(OC)cc(OC)c4[C@]2(O)[C@H](O)[C@H](CNCCN(C)CCN(C)CCN(C)CCN(C)CCN(C)CCN(C)CCN(C)CCN(C)CCN(C)CCN(C)CCN(C)CCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

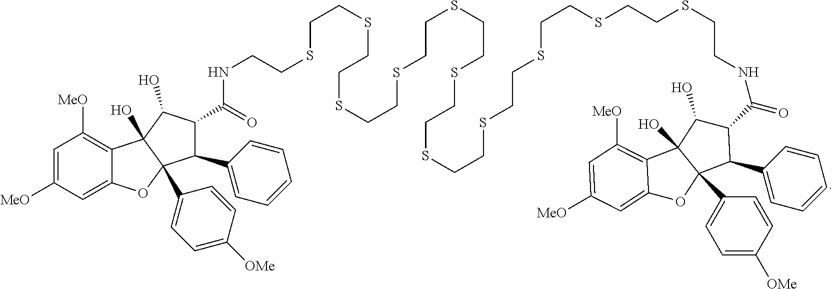 [SMILES: COc1ccc([C@@]23Oc4cc(OC)cc(OC)c4[C@]2(O)[C@H](O)[C@H](C(=O)NCCSCCSCCSCCSCCSCCSCCSCCSCCSCCSCCSCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

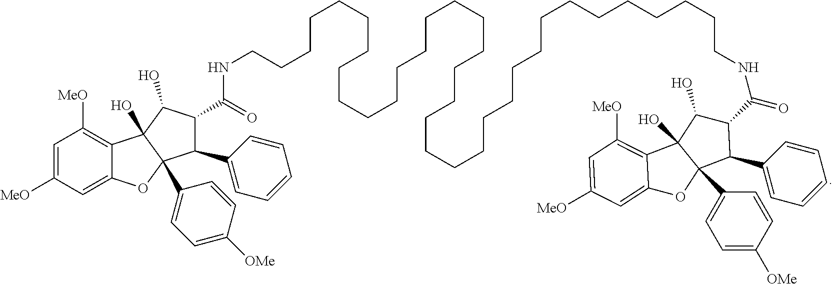 [SMILES: COc1ccc([C@@]23Oc4cc(OC)cc(OC)c4[C@]2(O)[C@H](O)[C@H](C(=O)NCCCCCCCCCCCCCCCCCCCCCCCCCCCCCCCCCCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

In embodiments, the compound has the formula:

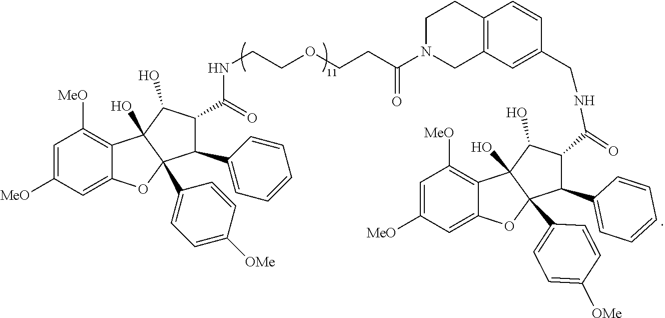

In embodiments, the compound has the formula:

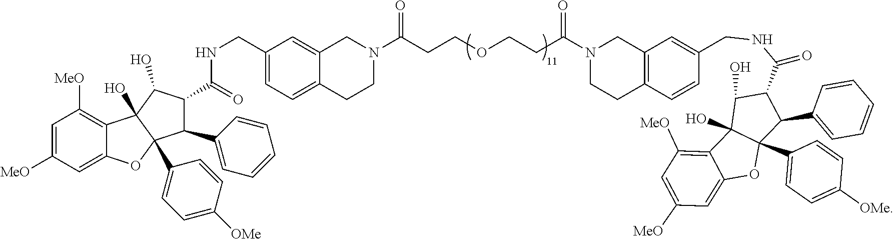

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{3A} and R^{3B} substituents are optionally combined to form a moiety that is substituted (e.g., a substituted alkenyl, substituted cycloalkyl, or substituted heterocycloalkyl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents are optionally combined to form a moiety that is substituted (e.g., a substituted alkenyl, substituted cycloalkyl, or substituted heterocycloalkyl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{4A} and R^{4B} substituents are optionally combined to form a moiety that is substituted (e.g., a substituted alkenyl, substituted cycloalkyl, or substituted heterocycloalkyl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents are optionally combined to form a moiety that is substituted (e.g., a substituted alkenyl, substituted cycloalkyl, or substituted heterocycloalkyl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R² and R^{3A} substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R² and R^{3A} substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{4A} substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{4A} substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{3B} and R^{4B} substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3B} and R^{4B} substituents bonded to the same nitrogen atom are optionally joined to form a moiety that is substituted (e.g., a substituted cycloalkyl or substituted heterocycloalkyl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4A} and R⁵ substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R⁵ substituents, together with the carbon atom to which they are attached, are optionally joined to form a moiety that is substituted (e.g., a substituted cycloalkyl or substituted heterocycloalkyl), the moiety is substituted with one or more first substituent groups denoted by R^{5.1} as explained in the definitions section above in the description of “first substituent group(s)”. In embodiments, when an R⁵¹ substituent group is substituted, the R⁵¹ substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R^{5.1}, R^{5.2}, and R^{5.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5.1}, R^{5.2}, and R^{5.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸, R^{8.1}, R^{8.2}, and R^{8.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2}, and R^{9.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when R^{9A} is substituted, R^{9A} is substituted with one or more first substituent groups denoted by R^{9A.1} as explained in the definitions section above in the description of “first substituent group(s)”. In embodiments, when an R^{9A.1} substituent group is substituted, the R^{9A.1} substituent group is substituted with one or more second substituent groups denoted by R^{9A.2} as explained in the definitions section above in the description of “first substituent group(s)”. In embodiments, when an R^{9A.2} substituent group is substituted, the R^{9A.2} substituent group is substituted with one or more third substituent groups denoted by R^{9A.3} as explained in the definitions section above in the description of “first substituent group(s)”. In the above embodiments, R^{9A}, R^{9A.1}, R^{9A.2}, and R^{9A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9A}, R^{9A.1}, R^{9A.2}, and R^{9A.3}, respectively.

In embodiments, when R^{9B} is substituted, R^{9B} is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B.3} as explained in the definitions section above in the description of “first substituent group(s)”. In the above embodiments, R^{9B}, R^{9B.1}, R^{9B.2}, and R^{9B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9B}, R^{9B.1}, R^{9B.2}, and R^{9B.3}, respectively.

In embodiments, when R^{9A} and R^{9B} substituents bonded to the same nitrogen atom are optionally joined to form a moiety that is substituted (e.g., a substituted heterocycloalkyl or substituted heteroaryl), the moiety is substituted with one or more first substituent groups denoted by R^{9A.1} as explained in the definitions section above in the description of “first substituent group(s)”. In embodiments, when an R^{9A.1} substituent group is substituted, the R^{9A.1} substituent group is substituted with one or more second substituent groups denoted by R^{9A.2} as explained in the definitions section above in the description of “first substituent group(s)”. In embodiments, when an R^{9A.2} substituent group is substituted, the R^{9A.2} substituent group is substituted with one or more third substituent groups denoted by R^{9A.3} as explained in the definitions section above in the description of “first substituent group(s)”. In the above embodiments, R^{9A.1}, R^{9A.2}, and R^{9A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{9A.1}, R^{9A.2}, and R^{9A.3}, respectively.

In embodiments, when R^{9A} and R^{9B} substituents bonded to the same nitrogen atom are optionally joined to form a moiety that is substituted (e.g., a substituted heterocycloalkyl or substituted heteroaryl), the moiety is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B.3} as explained in the definitions section above in the description of “first substituent group(s)”. In the above embodiments, R^{9B.1}, R^{9B.2}, and R^{9B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{9B.1}, R^{9B.2}, and R^{9B.3}, respectively.

In embodiments, when R^{9C} is substituted, R^{9C} is substituted with one or more first substituent groups denoted by R^{9C.1} as explained in the definitions section above in the description of “first substituent group(s)”. In embodiments, when an R^{9C.1} substituent group is substituted, the R^{9C.1} substituent group is substituted with one or more second substituent groups denoted by R^{9C.2} as explained in the definitions section above in the description of “first substituent group(s)”. In embodiments, when an R^{9C.2} substituent group is substituted, the R^{9C.2} substituent group is substituted with one or more third substituent groups denoted by R^{9C.3} as explained in the definitions section above in the description of “first substituent group(s)”. In the above embodiments, R^{9C}, R^{9C.1}, R^{9C.2}, and R^{9C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9C}, R^{9C.1}, R^{9C.2}, and R^{9C.3}, respectively.

In embodiments, when R^{9D} is substituted, R^{9D} is substituted with one or more first substituent groups denoted by R^{9D.1} as explained in the definitions section above in the description of “first substituent group(s)”. In embodiments, when an R^{9D.1} substituent group is substituted, the R^{9D.1} substituent group is substituted with one or more second substituent groups denoted by R^{9D.2} as explained in the definitions section above in the description of “first substituent group(s)”. In embodiments, when an R^{9D.2} substituent group is substituted, the R^{9D.2} substituent group is substituted with one or more third substituent groups denoted by R^{9D.3} as explained in the definitions section above in the description of “first substituent group(s)”. In the above embodiments, R^{9D}, R^{9D.1}, R^{9D.2}, and R^{9D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9D}, R^{9D.1}, R^{9D.2}, and R^{9D.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2}, and R^{10A.3}, respectively.

In embodiments, when R^{10B} is substituted, R^{10B} is substituted with one or more first substituent groups denoted by R^{10B} as explained in the definitions section above in the description of “first substituent group(s)”. In embodiments, when an R^{10B} substituent group is substituted, the R^{10B} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3}, respectively.

In embodiments, when R¹⁰¹ is substituted, R¹⁰¹ is substituted with one or more first substituent groups denoted by R^{101.1} as explained in the definitions section above in the description of “first substituent group(s)”. In embodiments, when an R^{101.1} substituent group is substituted, the R^{101.1} substituent group is substituted with one or more second substituent groups denoted by R^{101.2} as explained in the definitions section above in the description of “first substituent group(s)”. In embodiments, when an R^{101.2} substituent group is substituted, the R^{101.2} substituent group is substituted with one or more third substituent groups denoted by R^{101.3} as explained in the definitions section above in the description of “first substituent group(s)”. In the above embodiments, R¹⁰¹, R^{101.1}, R^{101.2}, and R^{101.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰¹, R^{101.1}, R^{101.2} and R^{101.3}, respectively.

In embodiments, when R¹⁰² is substituted, R¹⁰² is substituted with one or more first substituent groups denoted by R^{102.1} as explained in the definitions section above in the description of “first substituent group(s)”. In embodiments, when an R^{102.1} substituent group is substituted, the R^{102.1} substituent group is substituted with one or more second substituent groups denoted by R^{102.2} as explained in the definitions section above in the description of “first substituent group(s)”. In embodiments, when an R^{102.2} substituent group is substituted, the R^{102.2} substituent group is substituted with one or more third substituent groups denoted by R^{102.3} as explained in the definitions section above in the description of “first substituent group(s)”. In the above embodiments, R¹⁰², R^{102.1}, R^{102.2}, and R^{102.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰², R^{102.1}, R^{102.2}, and R^{102.3} respectively.

In embodiments, when R¹⁰³ is substituted, R¹⁰³ is substituted with one or more first substituent groups denoted by R^{103.1} as explained in the definitions section above in the description of “first substituent group(s)”. In embodiments, when an R^{103.1} substituent group is substituted, the R^{103.1} substituent group is substituted with one or more second substituent groups denoted by R^{103.2} as explained in the definitions section above in the description of “first substituent group(s)”. In embodiments, when an R^{103.2} substituent group is substituted, the R^{103.2} substituent group is substituted with one or more third substituent groups denoted by R^{103.3} as explained in the definitions section above in the description of “first substituent group(s)”. In the above embodiments, R¹⁰³, R^{103.1}, R^{103.2}, and R^{103.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰³, R^{103.1}, R^{103.2} and R^{103.3} respectively.

In embodiments, when L^{1A} is substituted, L^{1A} is substituted with one or more first substituent groups denoted by R^{L1A.1} as explained in the definitions section above in the description of “first substituent group(s)”. In embodiments, when an R^{L1A.1} substituent group is substituted, the R^{L1A.1} substituent group is substituted with one or more second substituent groups denoted by R^{L1A.2} as explained in the definitions section above in the description of “first substituent group(s)”. In embodiments, when an R^{L1A.2} substituent group is substituted, the R^{L1A.2} substituent group is substituted with one or more third substituent groups denoted by R^{L1A.3} as explained in the definitions section above in the description of “first substituent group(s)”. In the above embodiments, L^{1A}, R^{L1A.1}, R^{L1A.2}, and R^{L1A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1A}, R^{L1A.1}, R^{L1A.2}, and R^{L1A.3}, respectively.

In embodiments, when L^{1B} is substituted, L^{1B} is substituted with one or more first substituent groups denoted by R^{L1B.1} as explained in the definitions section above in the description of “first substituent group(s)”. In embodiments, when an R^{L1B.1} substituent group is substituted, the R^{L1B.1} substituent group is substituted with one or more second substituent groups denoted by R^{L1B.2} as explained in the definitions section above in the description of “first substituent group(s)”. In embodiments, when an R^{L1B.2} substituent group is substituted, the R^{L1B.2} substituent group is substituted with one or more third substituent groups denoted by R^{L1B.3} as explained in the definitions section above in the description of “first substituent group(s)”. In the above embodiments, L^{1B}, R^{L1B.1}, R^{L1B.2}, and R^{L1B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1B}, R^{L1B.1}, R^{L1B.2}, and R^{L1B.3}, respectively.

In embodiments, when L¹⁰¹ is substituted, L¹⁰¹ is substituted with one or more first substituent groups denoted by R^{L101.1} as explained in the definitions section above in the description of “first substituent group(s)”. In embodiments, when an R^{L101.1} substituent group is substituted, the R^{L101.1} substituent group is substituted with one or more second substituent groups denoted by R^{L101.2} as explained in the definitions section above in the description of “first substituent group(s)”. In embodiments, when an R^{L101.2} substituent group is substituted, the R^{L101.2} substituent group is substituted with one or more third substituent groups denoted by R^{L101.3} as explained in the definitions section above in the description of “first substituent group(s)”. In the above embodiments, L¹⁰¹, R^{L101.1}, R^{L101.2}, and R^{L101.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰¹, R^{L101.1}, R^{L101.2}, and R^{L101.3}, respectively.

In embodiments, when L¹⁰² is substituted, L¹⁰² is substituted with one or more first substituent groups denoted by R^{L102.1} as explained in the definitions section above in the description of “first substituent group(s)”. In embodiments, when an R^{L102.1} substituent group is substituted, the R^{L102.1} substituent group is substituted with one or more second substituent groups denoted by R^{L102.2} as explained in the definitions section above in the description of “first substituent group(s)”. In embodiments, when an R^{L102.2} substituent group is substituted, the R^{L102.2} substituent group is substituted with one or more third substituent groups denoted by R^{L102.3} as explained in the definitions section above in the description of “first substituent group(s)”. In the above embodiments, L¹⁰², R^{L102.1}, R^{L102.2}, and R^{L102.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰², R^{L102.1}, R^{L102.2}, and R^{L102.3}, respectively.

In embodiments, when L¹⁰³ is substituted, L¹⁰³ is substituted with one or more first substituent groups denoted by R^{L103.1} as explained in the definitions section above in the description of “first substituent group(s)”. In embodiments, when an R^{L103.1} substituent group is substituted, the R^{L103.1} substituent group is substituted with one or more second substituent groups denoted by R^{L103.2} as explained in the definitions section above in the description of “first substituent group(s)”. In embodiments, when an R^{L103.2} substituent group is substituted, the R^{L103.2} substituent group is substituted with one or more third substituent groups denoted by R^{L103.3} as explained in the definitions section above in the description of “first substituent group(s)”. In the above embodiments, L¹⁰³, R^{L103.1}, R^{L103.2}, and R^{L103.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰³, R^{L103.1}, R^{L103.2}, and R^{L103.3}, respectively.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (II), (III), or (IV). In embodiments, the compound is a compound of formula (II), (III), or (IV), including embodiments thereof.

### IV. Methods of Use

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is a MYC-amplified cancer. In embodiments, the cancer is an EGFR-amplified cancer. In embodiments, the cancer is a HER2-amplified cancer or a HER3-amplified cancer. In embodiments, the cancer is an FGFR1-amplified cancer or an FGFR2-amplified cancer. In embodiments, the cancer is an RTK-amplified cancer or a mutated RTK cancer. In embodiments, the cancer is a mutated KRAS cancer. In embodiments, the cancer is a cyclin D1-amplified cancer, a cyclin D2-amplified cancer, or a cyclin D3-amplified cancer. In embodiments, the cancer is a cancer with high phosphorylation of 4EBP1 or 4EBP2. In embodiments, the cancer is a cancer with loss of function of TSC1 or TSC2. In embodiments, the cancer is a cancer with loss of PTEN.

In embodiments, the cancer is bladder cancer, breast cancer, colorectal cancer, endometrial cancer, esophageal cancer, head and neck cancer, gastric cancer, glioblastoma, leukemia, liver cancer, lung cancer, lymphoma, neuroblastoma, ovarian cancer, pancreatic cancer, parathyroid adenoma, prostate cancer, renal cancer, skin cancer, thyroid cancer, or uterine cancer. In embodiments, the cancer is bladder cancer. In embodiments, the cancer is breast cancer. In embodiments, the breast cancer is triple negative breast cancer. In embodiments, the cancer is colorectal cancer. In embodiments, the cancer is endometrial cancer. In embodiments, the cancer is esophageal cancer. In embodiments, the esophageal cancer is esophageal adenocarcinoma. In embodiments, the cancer is head and neck cancer. In embodiments, the cancer is gastric cancer. In embodiments, the gastric cancer is gastric adenocarcinoma. In embodiments, the cancer is glioblastoma. In embodiments, the cancer is leukemia. In embodiments, the leukemia is acute myeloid leukemia. In embodiments, the cancer is liver cancer. In embodiments, the liver cancer is hepatocellular carcinoma. In embodiments, the cancer is lung cancer. In embodiments, the lung cancer is non-small cell lung cancer. In embodiments, the lung cancer is small cell lung cancer. In embodiments, the lung cancer is squamous cell lung carcinoma. In embodiments, the cancer is lymphoma. In embodiments, the cancer is multiple myeloma. In embodiments, the cancer is neuroblastoma. In embodiments, the cancer is ovarian cancer. In embodiments, the cancer is pancreatic cancer. In embodiments, the cancer is parathyroid adenoma. In embodiments, the cancer is prostate cancer. In embodiments, the cancer is renal cancer. In embodiments, the cancer is skin cancer. In embodiments, the skin cancer is melanoma. In embodiments, the skin cancer is squamous cell carcinoma. In embodiments, the cancer is thyroid cancer. In embodiments, the cancer is uterine cancer.

In embodiments, the method does not include further administering a second agent, wherein the second agent is an anti-cancer agent.

In an aspect is provided a method of modulating the level of activity of an eIF4A protein in a cell, the method including contacting the cell with an effective amount of a compound described herein, or a pharmaceutically acceptable salt thereof.

In embodiments, the modulating is reducing the activity of the eIF4A protein. In embodiments, the level of activity of the eIF4A protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the modulating is reducing the activity of the eIF4A protein. In embodiments, the level of activity of the eIF4A protein is reduced by about 2-fold relative to a control (e.g., absence of the compound). In embodiments, the modulating is reducing the activity of the eIF4A protein. In embodiments, the level of activity of the eIF4A protein is reduced by about 5-fold relative to a control (e.g., absence of the compound). In embodiments, the modulating is reducing the activity of the eIF4A protein. In embodiments, the level of activity of the eIF4A protein is reduced by about 10-fold relative to a control (e.g., absence of the compound). In embodiments, the modulating is reducing the activity of the eIF4A protein. In embodiments, the level of activity of the eIF4A protein is reduced by about 15-fold relative to a control (e.g., absence of the compound). In embodiments, the modulating is reducing the activity of the eIF4A protein. In embodiments, the level of activity of the eIF4A protein is reduced by about 20-fold relative to a control (e.g., absence of the compound). In embodiments, the modulating is reducing the activity of the eIF4A protein. In embodiments, the level of activity of the eIF4A protein is reduced by about 25-fold relative to a control (e.g., absence of the compound). In embodiments, the modulating is reducing the activity of the eIF4A protein. In embodiments, the level of activity of the eIF4A protein is reduced by about 30-fold relative to a control (e.g., absence of the compound). In embodiments, the modulating is reducing the activity of the eIF4A protein. In embodiments, the level of activity of the eIF4A protein is reduced by about 40-fold relative to a control (e.g., absence of the compound). In embodiments, the modulating is reducing the activity of the eIF4A protein. In embodiments, the level of activity of the eIF4A protein is reduced by about 50-fold relative to a control (e.g., absence of the compound). In embodiments, the modulating is reducing the activity of the eIF4A protein. In embodiments, the level of activity of the eIF4A protein is reduced by about 100-fold relative to a control (e.g., absence of the compound). In embodiments, the modulating is reducing the activity of the eIF4A protein. In embodiments, the level of activity of the eIF4A protein is reduced by about 200-fold relative to a control (e.g., absence of the compound). In embodiments, the modulating is reducing the activity of the eIF4A protein. In embodiments, the level of activity of the eIF4A protein is reduced by about 500-fold relative to a control (e.g., absence of the compound). In embodiments, the modulating is reducing the activity of the eIF4A protein. In embodiments, the level of activity of the eIF4A protein is reduced by about 1000-fold relative to a control (e.g., absence of the compound).

In embodiments, the level of activity of the eIF4A protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the eIF4A protein is reduced by at least 2-fold relative to a control (e.g., absence of the compound). In embodiments, the level of activity of the eIF4A protein is reduced by at least 5-fold relative to a control (e.g., absence of the compound). In embodiments, the level of activity of the eIF4A protein is reduced by at least 10-fold relative to a control (e.g., absence of the compound). In embodiments, the level of activity of the eIF4A protein is reduced by at least 15-fold relative to a control (e.g., absence of the compound). In embodiments, the level of activity of the eIF4A protein is reduced by at least 20-fold relative to a control (e.g., absence of the compound). In embodiments, the level of activity of the eIF4A protein is reduced by at least 30-fold relative to a control (e.g., absence of the compound). In embodiments, the level of activity of the eIF4A protein is reduced by at least 40-fold relative to a control (e.g., absence of the compound). In embodiments, the level of activity of the eIF4A protein is reduced by at least 50-fold relative to a control (e.g., absence of the compound). In embodiments, the level of activity of the eIF4A protein is reduced by at least 100-fold relative to a control (e.g., absence of the compound). In embodiments, the level of activity of the eIF4A protein is reduced by at least 200-fold relative to a control (e.g., absence of the compound). In embodiments, the level of activity of the eIF4A protein is reduced by at least 500-fold relative to a control (e.g., absence of the compound). In embodiments, the level of activity of the eIF4A protein is reduced by at least 1000-fold relative to a control (e.g., absence of the compound).

In embodiments, the eIF4A protein is a human eIF4A protein. In embodiments, the human eIF4A protein is a human eIF4A1 protein. In embodiments, the human eIF4A protein is a human eIF4A2 protein.

### V. Embodiments

Embodiment P1. A compound comprising a first eIF4A inhibitor attached to a second eIF4A inhibitor through a covalent linker.

Embodiment P2. The compound of embodiment P1, wherein the first eIF4A inhibitor and the second eIF4A inhibitor are the same.

Embodiment P3. The compound of embodiment P2, wherein the first eIF4A inhibitor and the second eIF4A inhibitor are a Rocaglate.

Embodiment P4. The compound of embodiment P2, wherein the first eIF4A inhibitor and the second eIF4A inhibitor are Rocaglamide A.

Embodiment P5. The compound of embodiment P2, wherein the first eIF4A inhibitor and the second eIF4A inhibitor are Zotatifin.

Embodiment P6. The compound of embodiment P2, wherein the first eIF4A inhibitor and the second eIF4A inhibitor are Silvestrol.

Embodiment P7. The compound of embodiment P2, wherein the first eIF4A inhibitor and the second eIF4A inhibitor are Pateamine A.

Embodiment P8. The compound of embodiment P2, wherein the first eIF4A inhibitor and the second eIF4A inhibitor are Hippuristanol.

Embodiment P9. The compound of embodiment P2, wherein the first eIF4A inhibitor and the second eIF4A inhibitor are a monovalent form of formula (I):

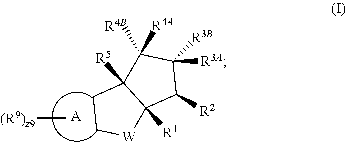

wherein
Ring A is aryl or heteroaryl;
W is CR⁶R⁷, O, S, NR′, C(O), C═CR⁶R⁷, N(CO)R⁸, S(O), or S(O)₂;
R¹ and R² are independently substituted or unsubstituted cycloalkyl, substituted or unsubstituted heterocycloalkyl, substituted or unsubstituted aryl, or substituted or unsubstituted heteroaryl;
R^{3A}, R^{3B}, R^{4A}, R^{4B}, R⁵, R⁶, and R⁷ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B}, or R^{4A} and R^{4B} independently combine to form oxo or substituted or unsubstituted alkenyl, substituted or unsubstituted cycloalkyl, or substituted or unsubstituted heterocycloalkyl;
R² and R^{3A}, R^{3A} and R^{4A}, R^{3B} and R^{4B}, or R^{4A} and R⁵, together with the carbon atom to which they are attached, form a substituted or unsubstituted cycloalkyl or substituted or unsubstituted heterocycloalkyl;
R⁸ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂ F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NR^{9C}NR^{9A}R^{9B}, —ONR^{9A}R^{9B}, —NHC(O)NR^{9A}R^{9B}, —N(O)ₘ₉, —NR^{9A}R^{9B}, —C(O)R^{9C}, —C(O)OR^{9C}, —C(O)NR^{9A}R^{9B}, —OR^{9D}, —SR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{9A} and R^{9B} substituents bonded to the same nitrogen atom may optionally be joined to form a substituted or unsubstituted heterocycloalkyl or substituted or unsubstituted heteroaryl;
X⁹ is independently —F, —Cl, —Br, or —I;
n9 is an integer from 0 to 4;
m9 and v9 are independently 1 or 2; and
z9 is an integer from 0 to 4.

Embodiment P10. The compound of embodiment P9, wherein R^{3A} is hydrogen.

Embodiment P11. The compound of one of embodiments P9 to P10, wherein R^{3B} is —C(O)NH₂ or substituted or unsubstituted 2 to 6 membered heteroalkyl.

Embodiment P12. The compound of one of embodiments P9 to P10, wherein R^{3B} is —C(O)NH₂ or —C(O)N(CH₃)₂.

Embodiment P13. The compound of embodiment P9, having the formula:

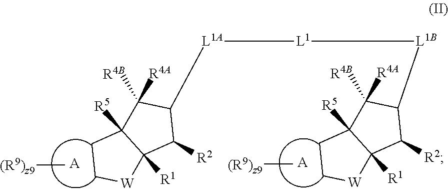

wherein
L¹ is said covalent linker;
L^{1A} is a bond, —C(O)—, —C(O)O—, —OC(O)—, —O—, —S—, —NR^{10A}—, —C(O)NR^{10A}—, —NR^{10A}C(O)—, —NR^{10A}C(O)O—, —OC(O)NR^{10A}—, —NR^{10A}C(O)NR^{10A}—, —NR^{10A}C(NH)NR^{10A}—, —S(O)₂—, —NR^{10A}S(O)₂—, —S(O)₂NR^{10A}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{1B} is a bond, —C(O)—, —C(O)O—, —OC(O)—, —O—, —S—, —NR^{10B}—, —C(O)NR^{10B}—, —NR^{10B}C(O)—, —NR^{10B}C(O)O—, —OC(O)NR^{10B}—, —NR^{10B}C(O)NR^{10B}—, —NR^{10B}C(NH)NR^{10B}—, —S(O)₂—, —NR^{10B}S(O)₂—, —S(O)₂NR^{10B}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R^{10A} and R^{10B} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P14. The compound of embodiment P13, wherein L^{1A} is —C(O)NR^{10A}— or substituted or unsubstituted 2 to 6 membered heteroalkylene.

Embodiment P15. The compound of embodiment P13, wherein L^{1A} is —C(O)NR^{10A}—, —C(O)NR^{10A}CH₂—, or —CH₂NHC(O)—.

Embodiment P16. The compound of embodiment P13, wherein L^{1A} is —C(O)NR^{10A}—.

Embodiment P17. The compound of one of embodiments P14 to P16, wherein R^{10A} is hydrogen or unsubstituted C₁-C₄ alkyl.

Embodiment P18. The compound of one of embodiments P14 to P16, wherein R^{10A} is hydrogen.

Embodiment P19. The compound of one of embodiments P13 to P18, wherein L^{1B} is —NR^{10B}C(O)— or substituted or unsubstituted 2 to 6 membered heteroalkylene.

Embodiment P20. The compound of one of embodiments P13 to P18, wherein L^{1B} is —NR^{10B}C(O)—, —CH₂NR^{10A}C(O)—, or —C(O)NHCH₂—.

Embodiment P21. The compound of one of embodiments P13 to P18, wherein L^{1B} is —NR^{10B}C(O)—.

Embodiment P22. The compound of one of embodiments P19 to P21, wherein R^{10B} is hydrogen or unsubstituted C₁-C₄ alkyl.

Embodiment P23. The compound of one of embodiments P19 to P21, wherein R^{10B} is hydrogen.

Embodiment P24. The compound of one of embodiments P9 to P23, wherein Ring A is phenyl or a 5 to 6 membered heteroaryl.

Embodiment P25. The compound of one of embodiments P9 to P23, wherein Ring A is phenyl.

Embodiment P26. The compound of one of embodiments P9 to P23, wherein Ring A is pyridyl.

Embodiment P27. The compound of one of embodiments P9 to P26, wherein W is O, S, NH, or C(O).

Embodiment P28. The compound of one of embodiments P9 to P26, wherein W is O.

Embodiment P29. The compound of one of embodiments P9 to P28, wherein R¹ and R² are independently substituted or unsubstituted phenyl.

Embodiment P30. The compound of one of embodiments P9 to P29, wherein R^{4A} is hydrogen.

Embodiment P31. The compound of one of embodiments P9 to P30, wherein R^{4B} is —OH.

Embodiment P32. The compound of one of embodiments P9 to P31, wherein R⁵ is —OH.

Embodiment P33. The compound of one of embodiments P9 to P32, wherein R⁹ is —OR^{9D}.

Embodiment P34. The compound of one of embodiments P9 to P32, wherein R⁹ is —OCH₃.

Embodiment P35. The compound of one of embodiments P9 to P34, wherein z9 is 2.

Embodiment P36. The compound of embodiment P1, having the formula:

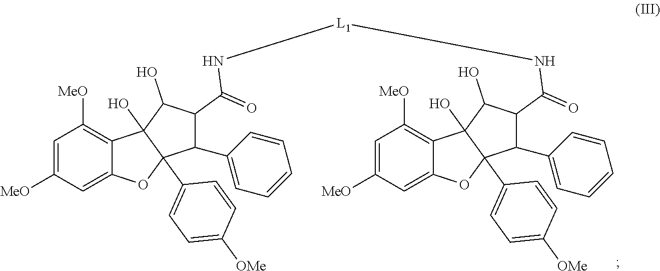

wherein L¹ is said covalent linker.

Embodiment P37. The compound of embodiment P1, having the formula:

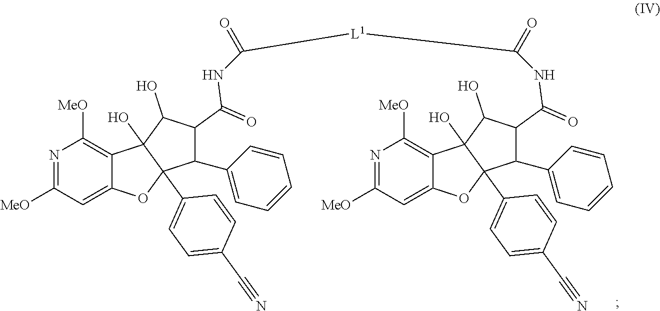

wherein L¹ is said covalent linker.

Embodiment P38. The compound of one of embodiments P1 to P37, wherein L¹ is said covalent linker; and
L¹ is -L¹⁰¹-L¹⁰²-L¹⁰³-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹¹⁰C(NH)NR¹⁰¹—, —S(O)₂—, —NR^{1O1}S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P39. The compound of one of embodiments P1 to P36, wherein L¹ is

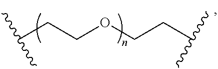

wherein n is an integer from 1 to 50.

Embodiment P40. The compound of one of embodiments P1 to P36, wherein L¹ is

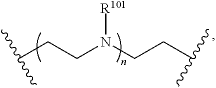

wherein R¹⁰¹ is independently hydrogen or unsubstituted C₁-C₄ alkyl; and n is an integer from 1 to 50.

Embodiment P41. The compound of one of embodiments P1 to P36, wherein L¹ is

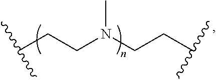

wherein n is an integer from 1 to 50.

Embodiment P42. The compound of one of embodiments P1 to P36, wherein L¹ is

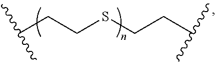

wherein n is an integer from 1 to 50.

Embodiment P43. The compound of one of embodiments P1 to P36, wherein L¹ is

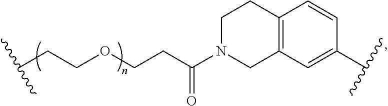

wherein n is an integer from 1 to 50.

Embodiment P43. The compound of one of embodiments P1 to P36, wherein L¹ is

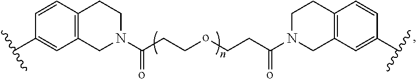

wherein n is an integer from 1 to 50.

Embodiment P45. The compound of one of embodiments P39 to P44, wherein n is an integer from 1 to 20.

Embodiment P46. The compound of one of embodiments P1 to P36, wherein L¹ is unsubstituted C₂-C₄₀ alkylene.

Embodiment P47. The compound of embodiment P1, having the formula:

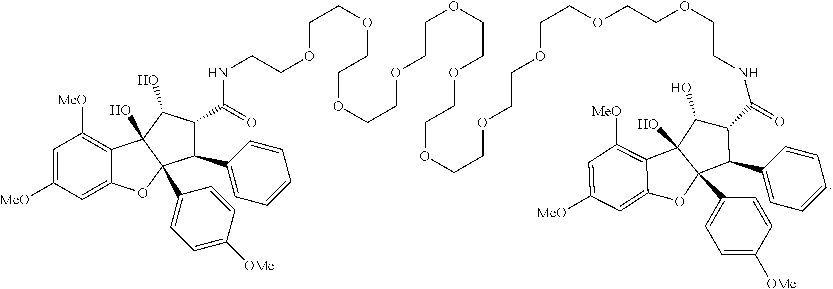 [SMILES: COc1ccc([C@@]23Oc4cc(OC)cc(OC)c4[C@]2(O)[C@H](O)[C@H](C(=O)NCCOCCOCCOCCOCCOCCOCCOCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

Embodiment P48. The compound of embodiment P1, having the formula:

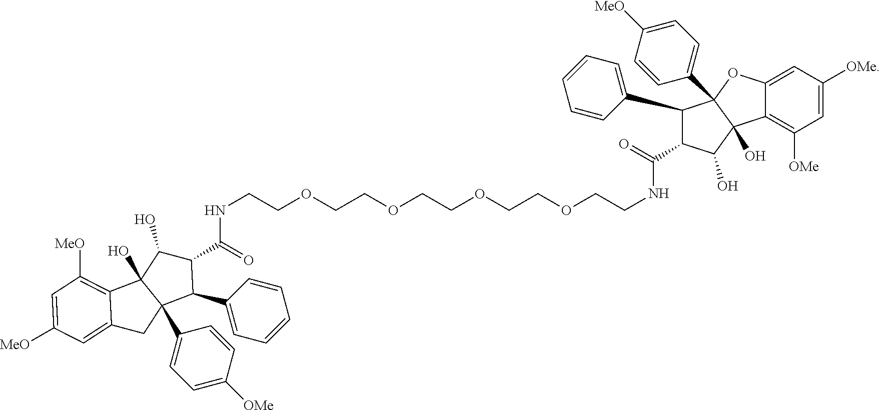 [SMILES: COc1ccc([C@@]23Cc4cc(OC)cc(OC)c4[C@]2(O)[C@H](O)[C@H](C(=O)NCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

Embodiment P49. The compound of embodiment P1, having the formula:

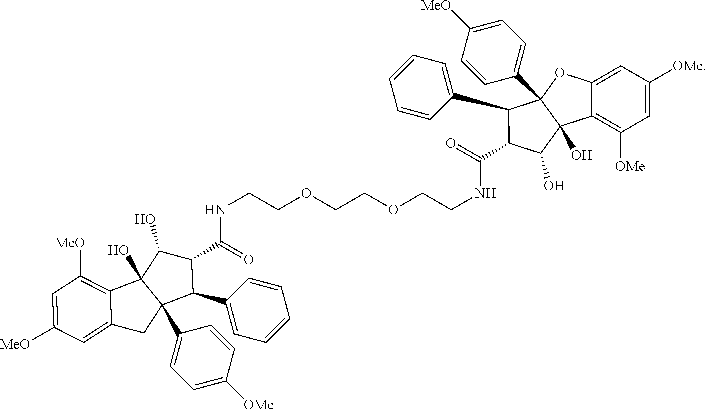 [SMILES: COc1ccc([C@@]23Cc4cc(OC)cc(OC)c4[C@]2(O)[C@H](O)[C@H](C(=O)NCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

Embodiment P50. A pharmaceutical composition comprising a pharmaceutically acceptable excipient and a compound of one of embodiments P1 to P49, or a pharmaceutically acceptable salt thereof.

Embodiment P51. A method of treating cancer in a subject in need thereof, said method comprising administering to the subject in need thereof a therapeutically effective amount of a compound of one of embodiments P1 to P49, or a pharmaceutically acceptable salt thereof.

Embodiment P52. The method of embodiment P51, wherein the cancer is a MYC-amplified cancer.

Embodiment P53. The method of embodiment P51, wherein the cancer is an EGFR-amplified cancer.

Embodiment P54. The method of embodiment P51, wherein the cancer is a HER2-amplified cancer or a HER3-amplified cancer.

Embodiment P55. The method of embodiment P51, wherein the cancer is an FGFR1-amplified cancer or an FGFR2-amplified cancer.

Embodiment P56. The method of embodiment P51, wherein the cancer is an RTK-amplified cancer or a mutated RTK cancer.

Embodiment P57. The method of embodiment P51, wherein the cancer is a mutated KRAS cancer.

Embodiment P58. The method of embodiment P51, wherein the cancer is a cyclin D1-amplified cancer, a cyclin D2-amplified cancer, or a cyclin D3-amplified cancer.

Embodiment P59. The method of embodiment P51, wherein the cancer is a cancer with high phosphorylation of 4EBP1 or 4EBP2.

Embodiment P60. The method of embodiment P51, wherein the cancer is a cancer with loss of function of TSC1 or TSC2.

Embodiment P61. The method of embodiment P51, wherein the cancer is a cancer with loss of PTEN.

Embodiment P62. The method of embodiment P51, wherein the cancer is bladder cancer, breast cancer, colorectal cancer, endometrial cancer, esophageal cancer, head and neck cancer, gastric cancer, glioblastoma, leukemia, liver cancer, lung cancer, lymphoma, multiple myeloma, neuroblastoma, ovarian cancer, pancreatic cancer, parathyroid adenoma, prostate cancer, renal cancer, skin cancer, thyroid cancer, or uterine cancer.

Embodiment P63. The method of embodiment P62, wherein the breast cancer is triple negative breast cancer.

Embodiment P64. The method of embodiment P62, wherein the esophageal cancer is esophageal adenocarcinoma.

Embodiment P65. The method of embodiment P62, wherein the gastric cancer is gastric adenocarcinoma.

Embodiment P66. The method of embodiment P62, wherein the leukemia is acute myeloid leukemia.

Embodiment P67. The method of embodiment P62, wherein the liver cancer is hepatocellular carcinoma.

Embodiment P68. The method of embodiment P62, wherein the lung cancer is non-small cell lung cancer, small cell lung cancer, or squamous cell lung carcinoma.

Embodiment P69. The method of embodiment P62, wherein the skin cancer is melanoma or squamous cell carcinoma.

Embodiment P70. A method of modulating the level of activity of an eIF4A protein in a cell, said method comprising contacting the cell with an effective amount of a compound of one of embodiments P1 to P49, or a pharmaceutically acceptable salt thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

Using complementary genome-scale chemical-genetic approaches, we identify, inter alia, an endogenous chemical uptake pathway involving interferon-induced transmembrane (IFITM) proteins that modulates the cell permeability of linked chemotypes, exemplified by a bitopic inhibitor of MTOR (RapaLink-1, molecular weight: 1784 g/mol). We devised additional linked inhibitors targeting EIF4A1 (BisRoc-1, molecular weight: 1466 g/mol) whose uptake we predicted would be assisted by IFITM proteins. This uptake pathway should provide a general mechanism by which large, flexibly linked chimeric molecules can gain assisted access to the cytosol, including compounds with mechanisms of action not easily accessible to traditional drug-like molecules.

Any therapeutic molecule that binds to an intracellular target must first navigate through the cell membrane. Retrospective analyses of compound libraries and their biological activities have yielded empirical guidelines (e.g., Lipinski's rule of five) that enrich for lead-like scaffolds with high passive permeability and largely define modern drug-like chemical space (1-3). While these principles have been useful for streamlining the search for novel therapeutics, many important intracellular drug targets are currently refractory to inhibition by these compact, hydrophobic, and rigid molecules. An emerging design framework that seeks to address these challenges involves increasing pharmacological complexity by linking multiple ligands into a single chemical entity (hereafter referred to as a linked chemotype). Doing so can imbue compounds with desirable properties such as enhanced potency (4), greater selectivity (4-6), and the capacity to induce the association of more than one target (7-10). These advances exemplify how high molecular weight, amphiphilicity, and rotational flexibility can enable rapid, modular access to useful chemical probes and therapeutic leads, as long as the resulting molecules remain cell permeable.

Mechanisms to understand and predict the cell permeability of linked chemotypes, however, remain limited. Other medium-to-high molecular weight therapeutics such as natural products and synthetic macrocycles often utilize highly tailored arrangements of polar/non-polar functionality to access membrane-favored and aqueous-favored conformations to enable their passive permeability through membranes (11). Additionally, cell penetrating proteins/peptides commonly require appendage of highly charged moieties to enable productive electrostatic interactions with the plasma membrane and subsequent internalization (12-14). Studies involving the most rapidly expanding linked chemotype in the literature, proteolysis targeting chimeras (PROTACs) (15), provide varying insights into the determinants of cell permeability (16-21), with one report finding no correlation between cell permeability and artificial membrane permeability (19). Despite their atypical properties, PROTACs and additional large molecules such as the dimeric immunophilin ligand rimiducid have shown in-cell activity robust enough to enter clinical trials (22) (NCT03888612; NCT04072952).

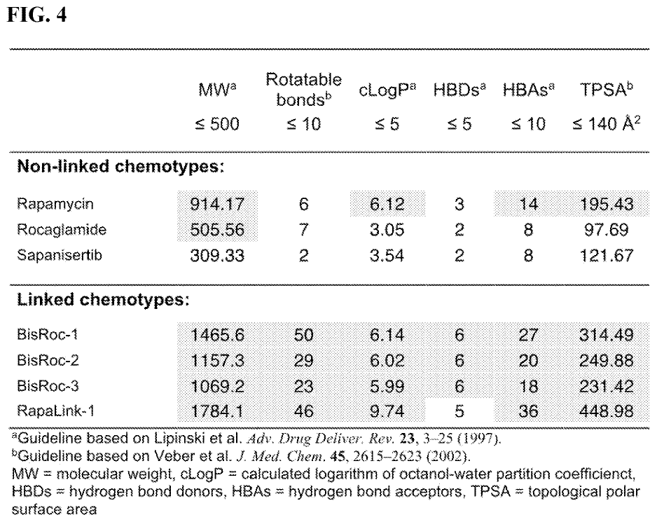

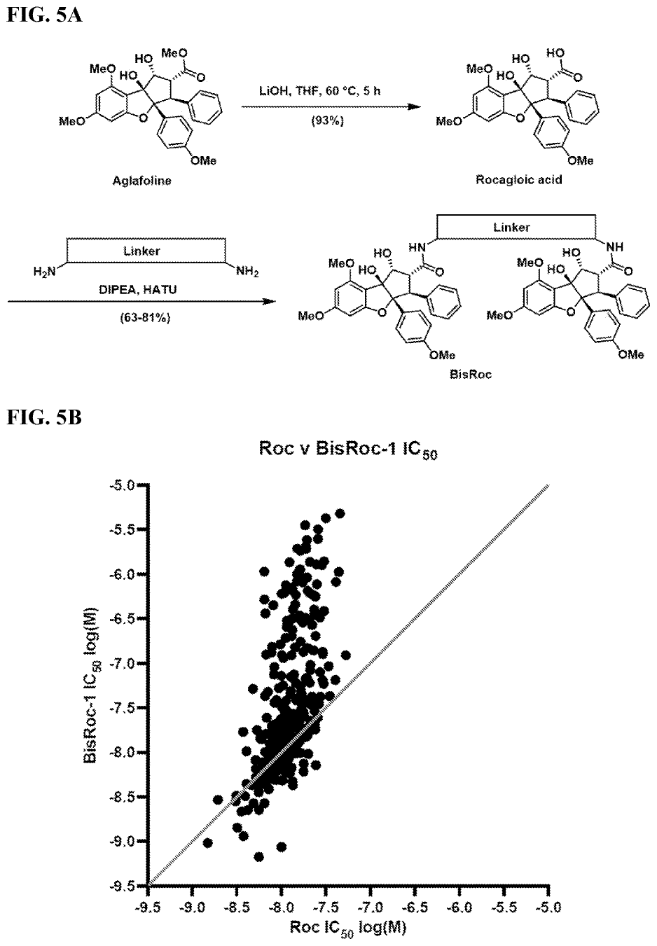

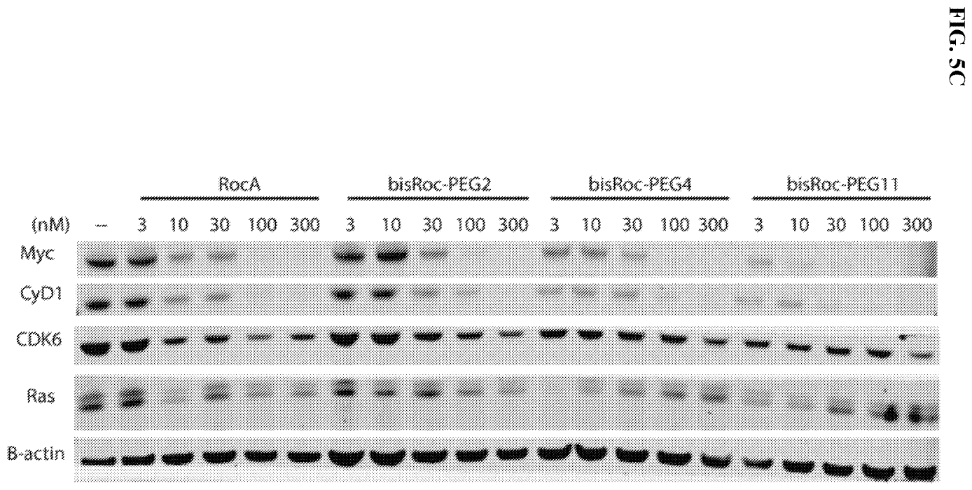

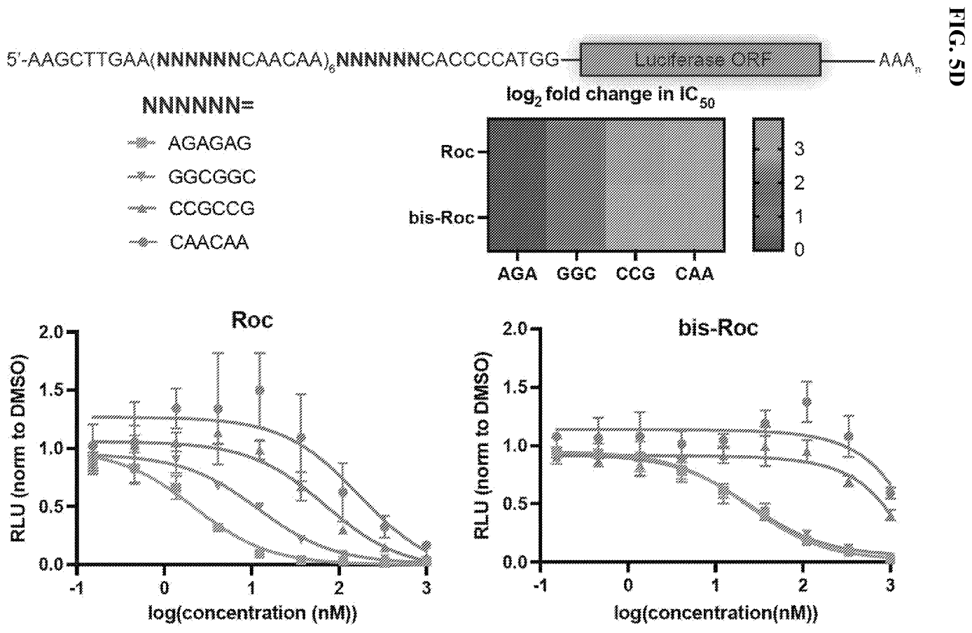

Given this discrepancy between the favorable biological activity of many large, bivalent molecules and traditional concepts of passive permeability, we inferred that linked chemotypes might hijack cellular processes to assist with passage through the cell membrane. We selected as an example a bitopic inhibitor of MTOR, RapaLink-1 (4), whose molecular weight (1784 g/mol) falls well beyond common guidelines (<500 g/mol) (1) and even beyond that of typical PROTACs (800-1200 g/mol) (FIG. 4) (19). RapaLink-1, which is composed of the allosteric MTOR inhibitor rapamycin and the active-site inhibitor sapanisertib linked by an 8-unit polyethylene glycol (PEG8) tether, is highly active in vivo (4, 5, 23), penetrates the blood-brain barrier (5, 23), and serves as a prototype for the clinical candidate RMC-5552 (NCT04774952), establishing itself as a drug-like compound that defies most traditional notions of drug-like structure. We hypothesized that cellular mechanisms assisting RapaLink-1's cytoplasmic entry could be identified by systematically perturbing genes that modulate the molecule's ability to reach and inhibit its intracellular target.

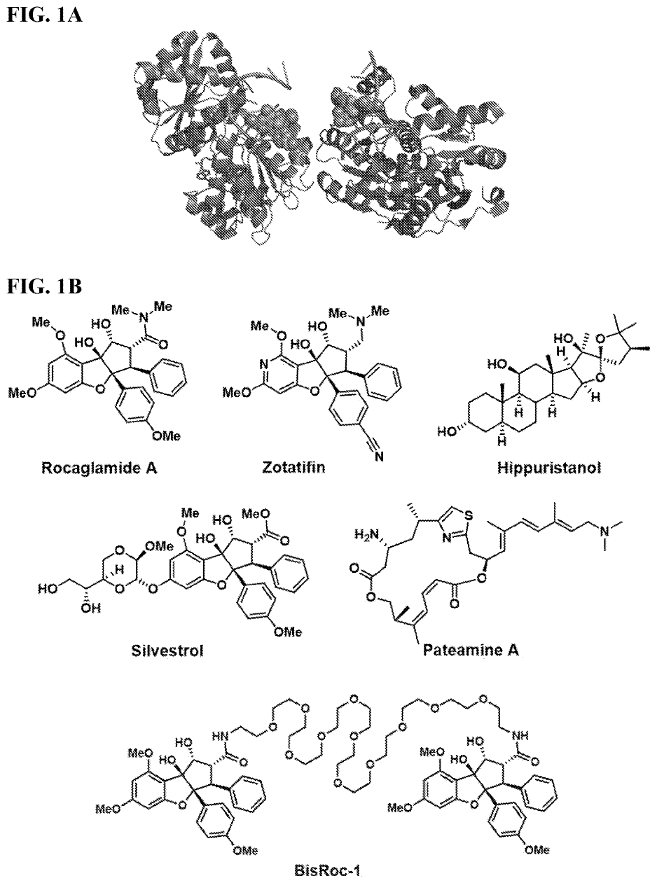

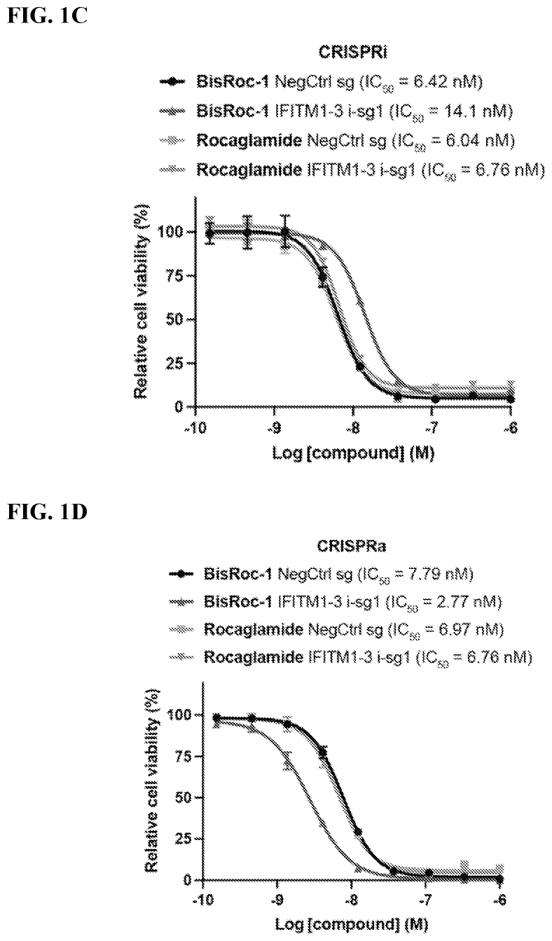

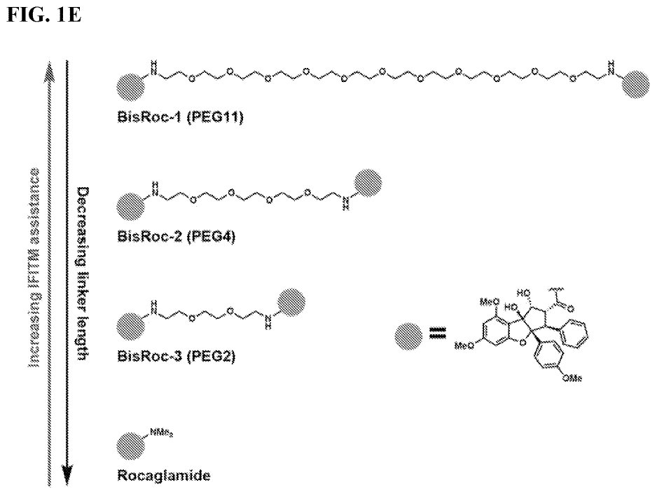

To further examine the breadth of linked chemotypes that might be assisted by IFITM proteins, we designed, synthesized, and characterized a new linked molecular glue inhibitor based on the natural product rocaglamide. Rocaglamide clamps the eIF4A1 helicase to 5′ untranslated regions (UTRs) of target mRNAs to inhibit the translation of downstream sequences (24). The crystal structure of the complex of rocaglamide, eIF4A1, and polypurine RNA (25) revealed that its eponymous amide points toward free solvent, near a symmetry mate (FIG. 1A). We reasoned that dimerization of rocaglamide through its amide position could be a chemically tractable means to simultaneously engage two proximal EIF4A1-RNA complexes within the cell. We designed a molecule, BisRoc-1 (FIG. 1), that links two rocaglamide monomers together with a linker length (35 heavy atoms) exceeding the distance separating two rocaglamide binding sites in the crystal structure (FIG. 1A). The linked and non-linked inhibitors displayed similar potencies toward K562 CRISPRi/a cell viability but diverged, as in prior examples, in their assistance by IFITM proteins, which accounted for an overall 6.2-fold modulation in the cellular activity of BisRoc-1 (FIG. 1C-1D). Combined, these data suggest the general feasibility of retaining cell permeability despite increased pharmacophore size, polarity, and flexibility in the context of linked chemotypes described herein.

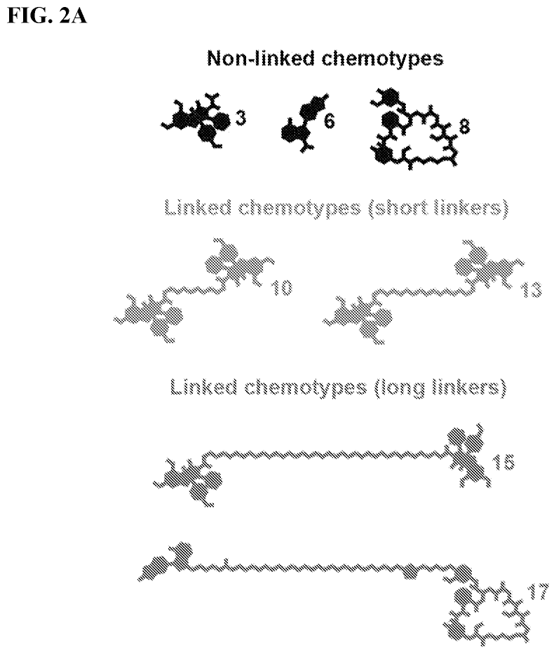

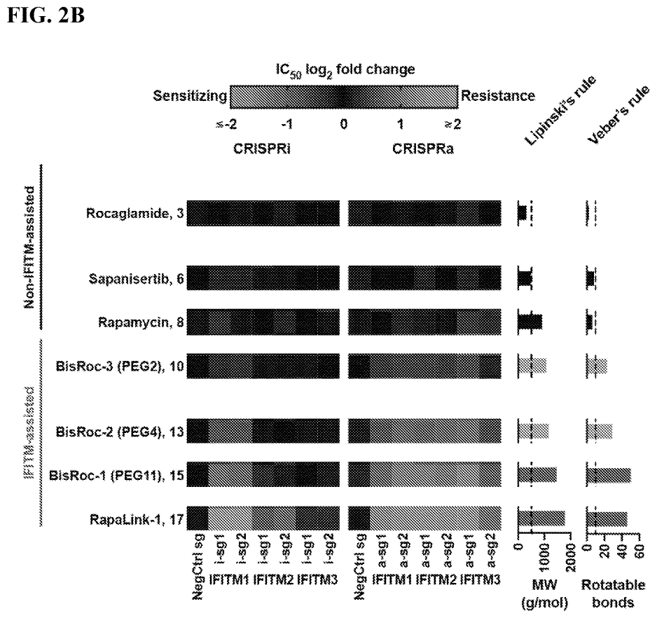

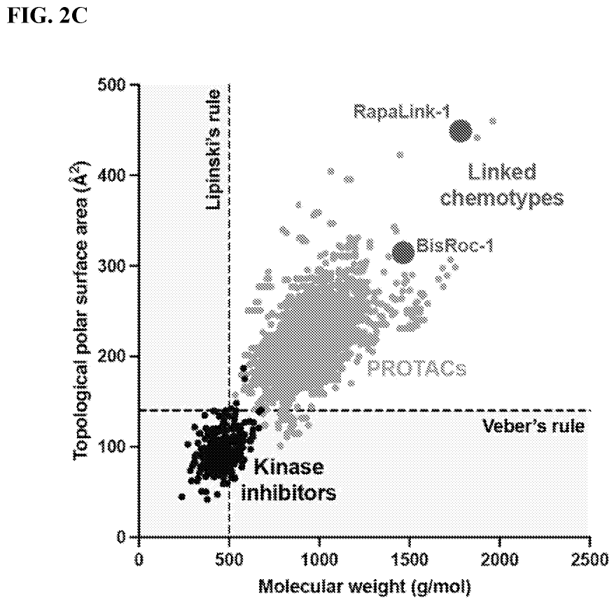

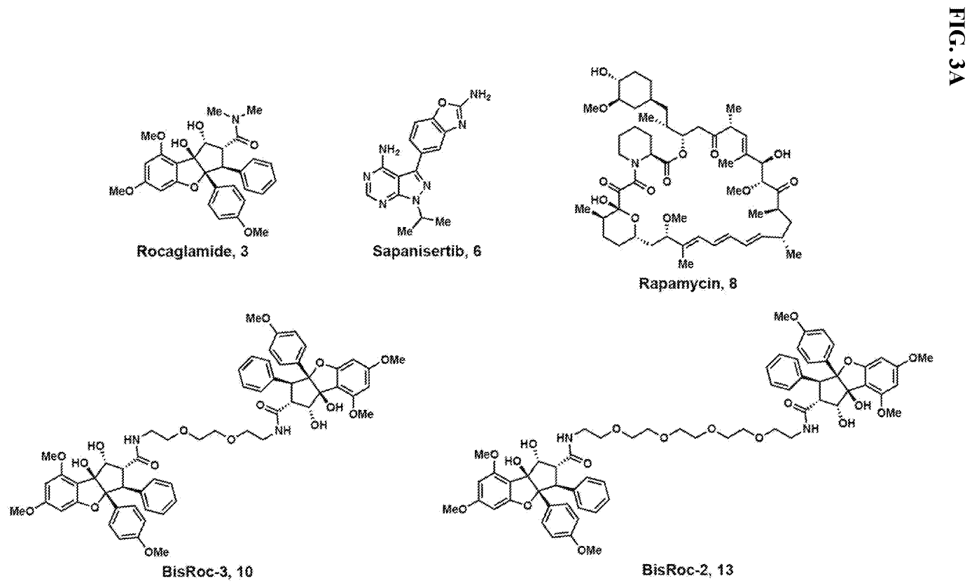

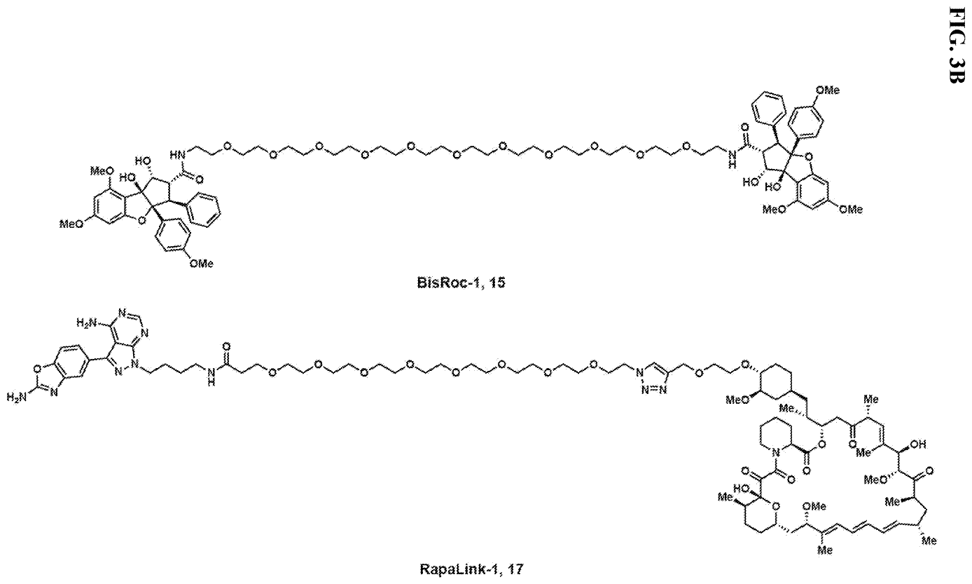

Given the ubiquitous presence of IFITM proteins in cells, we hypothesized that the cellular uptake of other linked inhibitors in the literature could also be assisted by IFITM proteins. While not generally as large as the linked chemotypes described above, PROTACs are likewise composed of two chemical entities covalently attached by a flexible tether (15). Incidentally, IFITM2 has been described as a statistically significant resistance chemical-genetic interaction with the PROTAC dBET6 in a separate CRISPR/Cas9 genome-scale knockout screen in KBM7 cells, although this result has not been validated or pursued to our knowledge (26). Thus, we included several PROTACs and their non-linked parent inhibitors in an expanded survey of chemical-genetic interactions with IFITM proteins (FIG. 2A, FIGS. 3A-3B, and FIG. 4). We treated our K562 CRISPRi and CRISPRa cells with these inhibitors and evaluated differences in potency resulting from IFITM protein expression modulation, as measured by half-maximal inhibitory concentration (IC₅₀) shift in a cell viability assay. Using RapaLink-1 as a chemical benchmark, we observed that IFITM1, IFITM2, and IFITM3 overexpression broadly sensitized cells to linked chemotypes (FIG. 2B; compounds 10, 13, 15, and 17). The inverse finding, resistance to linked chemotypes, resulted from gene knockdown (FIG. 2B). Included among these compounds were a systematic linker series of BisRoc-1 analogs (FIG. 1E). Data from BisRoc-1 (PEG11), BisRoc-2 (PEG4), BisRoc-3 (PEG2), and rocaglamide (no linker) revealed a pattern in which longer linker lengths correlated with greater IFITM assistance. The same trend was corroborated by the diverse ensemble of all molecules tested (FIG. 2B): the magnitudes of chemical-genetic interactions correlated with inhibitor size (molecular weight) and flexibility (number of rotatable bonds). Linked chemotypes with long linkers were more IFITM-assisted than linked chemotypes with short linkers, and non-linked chemotypes (FIG. 2B; compounds 3, 6, and 8) were not observed to be assisted by IFITM proteins (FIG. 2B). Despite their cellular activities, the physicochemical properties of these linked chemotypes largely violate Lipinski's (1) and Veber's (2) classic guidelines (FIG. 2B and FIG. 4), raising the need for a revised drug design framework that considers IFITM-assisted uptake and other cellular import processes. While a full characterization of the rules governing IFITM dependency will require further study, we propose that this uptake pathway can serve to generally assist the cellular entry of diverse large, flexible molecules of suitable amphiphilicity.

Through a combination of functional genomics and chemical methods, we uncovered an endogenous chemical uptake pathway involving IFITM proteins that appears to be harnessed by diverse linked chemotypes. With the clinical advancement of a dimeric immunophilin ligand (22), PROTACs (NCT03888612 and NCT04072952), and a RapaLink-1 derivative (NCT04774952), the notion of ‘drug-like’ is continually being revised. As evidence, the chemical space (11, 27) populated by an ever-expanding set of linked preclinical compounds in the literature ventures beyond that occupied by lead inhibitors developed under traditional guidelines (FIG. 2C) (1-3). The linked inhibitors RapaLink-1, DasatiLink-1, and BisRoc-1 reach even further past these boundaries (FIG. 2C), and the absolute limits to molecular size, polarity, and flexibility for cell permeable compounds have not yet been fully explored. Here, we identify IFITM-assisted cellular uptake as one of the mechanisms by which linked inhibitors are able to break previously established rules surrounding drug-likeness. We hope that the discovery of this uptake pathway will prompt further exploration of novel chemical space, and spur the development of compounds with mechanisms of action difficult to attain with compact, hydrophobic molecules.

### REFERENCES FOR EXAMPLE 1

1. C. A. Lipinski, F. Lombardo, B. W. Dominy, P. J. Feeney, Adv Drug Deliver Rev. 23, 3-25 (1997). 2. D. F. Veber, S. R. Johnson, H.-Y. Cheng, B. R. Smith, K. W. Ward, K. D. Kopple, J Med Chem. 45, 2615-2623 (2002). 3. A. K. Ghose, V. N. Viswanadhan, J. J. Wendoloski, J Comb Chem. 1, 55-68 (1999). 4. V. S. Rodrik-Outmezguine, M. Okaniwa, Z. Yao, C. J. Novotny, C. McWhirter, A. Banaji, H. Won, W. Wong, M. Berger, E. de Stanchina, D. G. Barratt, S. Cosulich, T. Klinowska, N. Rosen, K. M. Shokat, Nature. 534, 272-6 (2016). 5. Q. Fan, O. Aksoy, R. A. Wong, S. Ilkhanizadeh, C. J. Novotny, W. C. Gustafson, A. Y.-Q. Truong, G. Cayanan, E. F. Simonds, D. Haas-Kogan, J. J. Phillips, T. Nicolaides, M. Okaniwa, K. M. Shokat, W. A. Weiss, Cancer Cell. 31, 424-435 (2017). 6. F. A. Meijer, G. J. M. Oerlemans, L. Brunsveld, Acs Chem Biol. 16, 510-519 (2021). 7. D. Spencer, T. Wandless, S. Schreiber, G. Crabtree, Science. 262, 1019-1024 (1993). 8. K. M. Sakamoto, K. B. Kim, A. Kumagai, F. Mercurio, C. M. Crews, R. J. Deshaies, Proc National Acad Sci. 98, 8554-8559 (2001). 9. G. S. Erwin, M. P. Grieshop, A. Ali, J. Qi, M. Lawlor, D. Kumar, I. Ahmad, A. McNally, N. Teider, K. Worringer, R. Sivasankaran, D. N. Syed, A. Eguchi, Md. Ashraf, J. Jeffery, M. Xu, P. M. C. Park, H. Mukhtar, A. K. Srivastava, M. Faruq, J. E. Bradner, A. Z. Ansari, Science. 358, eaan6414 (2017). 10. M. G. Costales, Y. Matsumoto, S. P. Velagapudi, M. D. Disney, J Am Chem Soc. 140, 6741-6744 (2018). 11. B. C. Doak, B. Over, F. Giordanetto, J. Kihlberg, Chem Biol. 21, 1115-1142 (2014). 12. M. Green, P. M. Loewenstein, Cell. 55, 1179-1188 (1988). 13. A. D. Frankel, C. O. Pabo, Cell. 55, 1189-1193 (1988). 14. E. L. Snyder, S. F. Dowdy, Pharmaceut Res. 21, 389-393 (2004). 15. A. C. Lai, C. M. Crews, Nat Rev Drug Discov. 16, 101-114 (2017). 16. C. A. Foley, F. Potjewyd, K. N. Lamb, L. I James, S. V. Frye, Acs Chem Biol. 15, 290-295 (2019). 17. V. G. Klein, C. E. Townsend, A. Testa, M. Zengerle, C. Maniaci, S. J. Hughes, K.-H. Chan, A. Ciulli, R. S. Lokey, Acs Med Chem Lett. 11, 1732-1738 (2020). 18. D. E. Scott, T. P. C. Rooney, E. D. Bayle, T. Mirza, H. M. G. Willems, J. H. Clarke, S. P. Andrews, J. Skidmore, Acs Med Chem Lett (2020), doi:10.1021/acsmedchemlett.0c00194. 19. C. Cantrill, P. Chaturvedi, C. Rynn, J. P. Schaffland, I. Walter, M. B. Wittwer, Drug Discov Today. 25, 969-982 (2020). 20. G. Ermondi, M. Vallaro, G. Caron, Drug Discov Today. 25, 1585-1591 (2020). 21. Y. Atilaw, V. Poongavanam, C. S. Nilsson, D. Nguyen, A. Giese, D. Meibom, M. Erdelyi, J. Kihlberg, Acs Med Chem Lett. 12, 107-114 (2020). 22. J. D. Iuliucci, S. D. Oliver, S. Morley, C. Ward, J. Ward, D. Dalgarno, T. Clackson, H. J. Berger, Intravenous Safety and Pharmacokinetics of a Novel Dimerizer Drug, AP1903, in Healthy Volunteers (2001), pp. 870-879. 23. Z. Zhang, Q. Fan, X. Luo, K. J. Lou, W. A. Weiss, K. M. Shokat, Biorxiv, in press, doi:10.1101/2020.10.12.336677. 24. S. Iwasaki, S. N. Floor, N. T. Ingolia, Nature. 534, 558 (2016). 25. S. Iwasaki, W. Iwasaki, M. Takahashi, A. Sakamoto, C. Watanabe, Y. Shichino, S. N. Floor, K. Fujiwara, M. Mito, K. Dodo, M. Sodeoka, H. Imataka, T. Honma, K. Fukuzawa, T. Ito, N. T. Ingolia, Mol Cell. 73, 738-748.e9 (2019). 26. C. Mayor-Ruiz, M. G. Jaeger, S. Bauer, M. Brand, C. Sin, A. Hanzl, A. C. Mueller, J. Menche, G. E. Winter, Mol Cell. 75, 849-858.e8 (2019). 27. S. L. Schreiber, Science. 287, 1964-1969 (2000).

### Example 2: Experimental Methods

### Chemical Synthesis

Nuclear magnetic resonance (NMR) spectra were recorded on a Bruker spectrometer at 400 MHz. Chemical shifts were reported as parts per million (ppm) from solvent references. Liquid chromatography-mass spectrometry (LC-MS) was performed on a Waters Xevo G2-XS QTof (0.6 mL/min) using an ACQUITY UPLC BEH C18 column (Waters) and a water/acetonitrile gradient (0.05% formic acid) using Optima LC-MS grade solvents (Fisher Scientific). All other solvents (Fisher Scientific, Millipore Sigma) and commercially available reagents were used without further purification. Analytical thin-layer chromatography was performed with silica gel 60 F254 glass plates (Millipore Sigma). Flash chromatography was performed with RediSep Rf normal-phase silica flash columns using a CombiFlash Rf+(Teledyne ISCO).

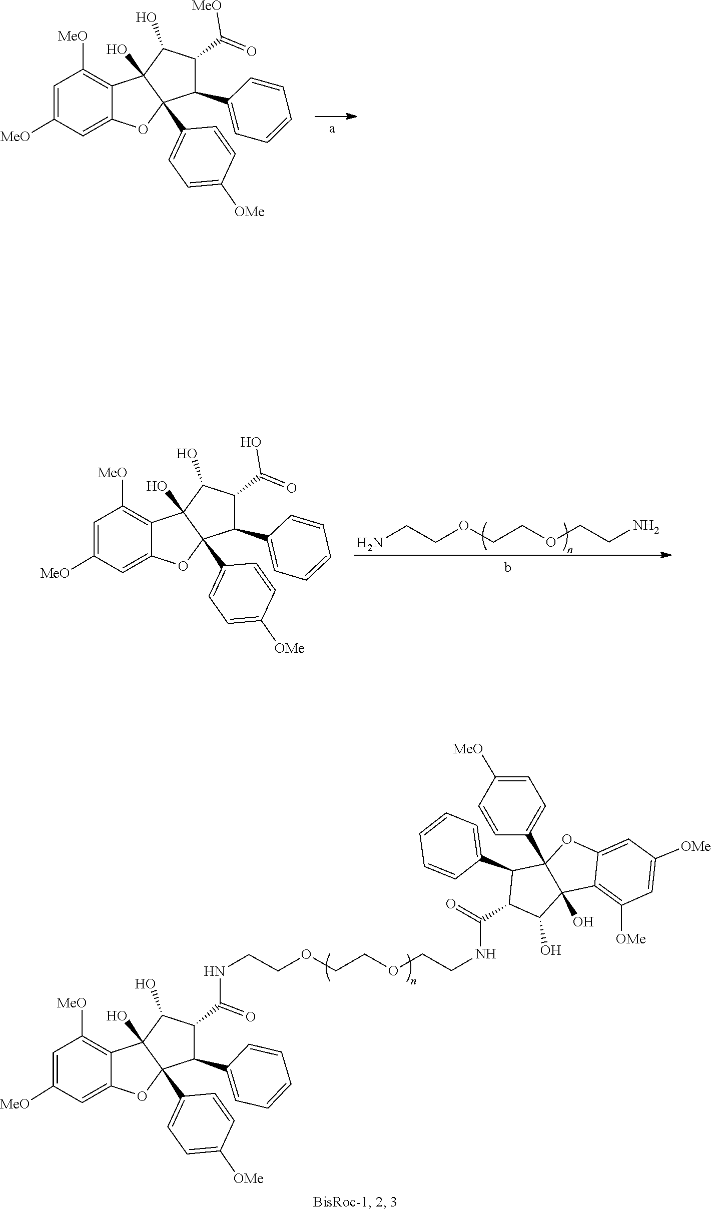

Reagents and conditions. (a) Aqueous LiOH, THF, 60 °C, 93%. (b) HATU, DIPEA, DMF, rt, 63-81%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide.

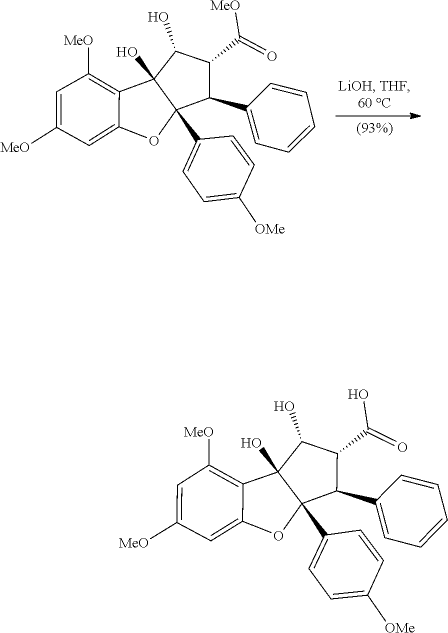 [SMILES: COC(=O)[C@H]1[C@@H](O)[C@@]2(O)c3c(OC)cc(OC)cc3O[C@@]2(c2ccc(OC)cc2)[C@@H]1c1ccccc1 ; COc1ccc([C@@]23Oc4cc(OC)cc(OC)c4[C@]2(O)[C@H](O)[C@H](C(=O)O)[C@H]3c2ccccc2)cc1]

Rocagloic acid. This protocol was adapted from Lajkiewicz et al., J. Am. Chem. Soc. 136, 2659-2664 (2014). To a mixture of aglafoline (73 mg, 0.148 mmol) in THF (3.7 mL) was added 0.1 M lithium hydroxide in water (3.7 mL, 0.371 mmol). The reaction was stirred at 60 °C for 5 h. After allowing the reaction to cool to room temperature, the mixture was partitioned between dichloromethane and 5% citric acid in water. The aqueous layer was extracted with dichloromethane (4×) and the combined organics were washed with brine (2×), dried over sodium sulfate, filtered, and concentrated in vacuo. The crude was purified by flash chromatography over silica gel eluting with a gradient from 0% methanol-dichloromethane to 20% methanol-dichloromethane to afford rocagloic acid (66 mg, 0.138 mmol, 93% yield) as a white solid.

¹H NMR (400 MHz, DMSO-d₆) δ 12.05 (s, 1H), 7.08-6.92 (m, 5H), 6.88 (d, J=7.2 Hz, 2H), 6.58 (d, J=9.0 Hz, 2H), 6.27 (d, J=1.9 Hz, 1H), 6.11 (d, J=2.0 Hz, 1H), 5.01 (s, 1H), 4.95 (s, 1H), 4.67 (d, J=5.7 Hz, 1H), 4.10 (d, J=14.0 Hz, 1H), 3.79 (dd, J=14.2, 5.7 Hz, 1H), 3.78 (s, 3H), 3.74 (s, 3H), 3.60 (s, 3H). ¹³C NMR (100 MHz, DMSO-d₆) δ 171.2, 162.6, 160.5, 157.8, 157.4, 138.7, 128.7, 128.7 (2C), 127.7 (2C), 127.3 (2C), 125.7, 111.8 (2C), 108.4, 101.4, 93.3, 91.8, 88.4, 78.8, 55.5, 55.4, 54.7, 54.7, 50.9. HRMS (m/z): calculated for C₂₇H₂₇O₈⁺ [M+H]⁺ 479.1700, found 479.1706. TLC: R_{f}=0.6 (20% methanol-dichloromethane).

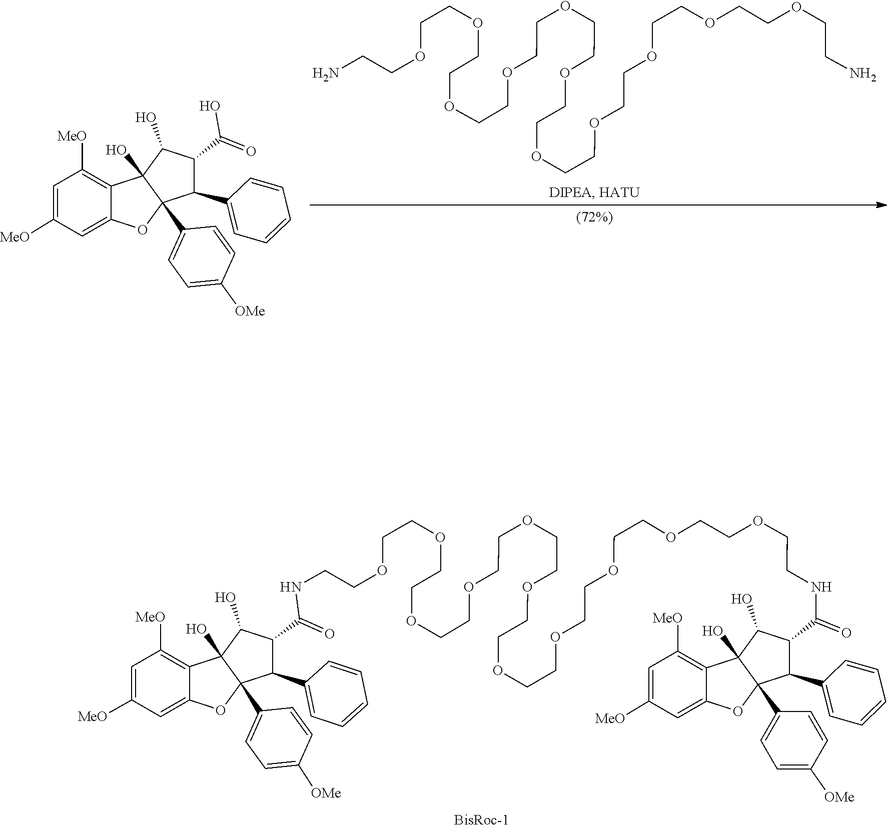 [SMILES: COc1ccc([C@@]23Oc4cc(OC)cc(OC)c4[C@]2(O)[C@H](O)[C@H](C(=O)O)[C@H]3c2ccccc2)cc1 ; NCCOCCOCCOCCOCCOCCOCCOCCOCCOCCOCCOCCN ; COc1ccc([C@@]23Oc4cc(OC)cc(OC)c4[C@]2(O)[C@H](O)[C@H](C(=O)NCCOCCOCCOCCOCCOCCOCCOCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

BisRoc-1. To a mixture of rocagloic acid (30 mg, 0.0627 mmol) and amino-PEG11-amine (17.1 mg, 0.0314 mmol) in N,N-dimethylformamide (0.627 mL) was added N,N-diisopropylethylamine (55 μL, 0.313 mmol). The solution was cooled in an ice-water bath before the addition of 1-[bis(dimethylamino)methylene]-1H-1,2,3-triazolo[4,5-b]pyridinium 3-oxide hexafluorophosphate (26 mg, 0.0684 mmol) and stirred at room temperature overnight. The mixture was partitioned between ethyl acetate and water and the organic layer was washed with water (4×) and brine (2×), dried over sodium sulfate, filtered, and concentrated in vacuo. The crude was purified by flash chromatography over silica gel eluting with a gradient from 0% methanol-dichloromethane to 10% methanol-dichloromethane to afford BisRoc-1 (33 mg, 0.0225 mmol, 72% yield) as a white solid.

¹H NMR (400 MHz, DMSO-d₆) δ 8.39-8.19 (m, 2H), 7.10-6.99 (m, 8H), 6.99-6.88 (m, 6H), 6.59 (d, J=8.9 Hz, 4H), 6.27 (d, J=1.9 Hz, 2H), 6.10 (d, J=2.0 Hz, 2H), 4.94 (s, 2H), 4.59-4.50 (m, 4H), 4.17 (d, J=14.1 Hz, 2H), 3.85 (dd, J=14.1, 5.0 Hz, 2H), 3.77 (s, 6H), 3.73 (s, 6H), 3.60 (s, 6H), 3.53-3.43 (m, 40H), 3.37-3.27 (m, 4H), 3.23-3.08 (m, 4H). ¹³C NMR (100 MHz, DMSO-d₆) δ 170.3 (2C), 162.6 (2C), 160.6 (2C), 157.9 (2C), 157.4 (2C), 138.5 (2C), 128.9 (2C), 128.7 (4C), 127.9 (4C), 127.3 (4C), 125.7 (2C), 111.7 (4C), 108.5 (2C), 101.3 (2C), 93.3 (2C), 91.7 (2C), 88.4 (2C), 78.9 (2C), 69.8 (16C), 69.7 (2C), 69.6 (2C), 69.1 (2C), 55.5 (2C), 55.4 (2C), 55.3 (2C), 54.7 (2C), 50.0 (2C), 38.6 (2C). HRMS (m z): calculated for C₇₈H₁₀₁N₂O₂₅⁺ [M+H]⁺ 1465.6688, found 1465.6643. TLC: R_{f}=0.2 (10% methanol-dichloromethane).

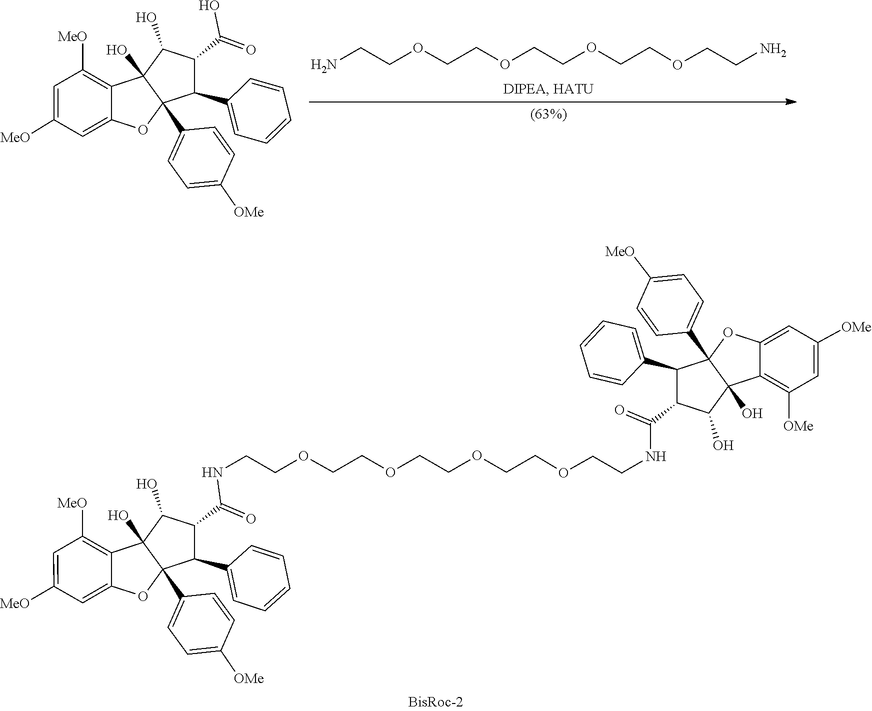 [SMILES: COc1ccc([C@@]23Oc4cc(OC)cc(OC)c4[C@]2(O)[C@H](O)[C@H](C(=O)O)[C@H]3c2ccccc2)cc1 ; NCCOCCOCCOCCOCCN ; COc1ccc([C@@]23Oc4cc(OC)cc(OC)c4[C@]2(O)[C@H](O)[C@H](C(=O)NCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

BisRoc-2. The same procedure as for BisRoc-1, using amino-PEG4-amine as starting material with scaled reagents, afforded BisRoc-2 (23 mg, 0.0199 mmol, 63% yield) as a white solid.

¹H NMR (400 MHz, DMSO-d₆) δ 8.40-8.19 (m, 2H), 7.11-6.99 (m, 8H), 6.99-6.88 (m, 6H), 6.59 (d, J=9.0 Hz, 4H), 6.27 (d, J=2.0 Hz, 2H), 6.10 (d, J=2.0 Hz, 2H), 4.95 (s, 2H), 4.54 (d, J=5.3 Hz, 4H), 4.17 (d, J=14.1 Hz, 2H), 3.85 (dd, J=14.2, 5.3 Hz, 2H), 3.77 (s, 6H), 3.73 (s, 6H), 3.60 (s, 6H), 3.51 (s, 4H), 3.50-3.47 (m, 4H), 3.47-3.43 (m, 4H), 3.39-3.26 (m, 4H), 3.22-3.09 (m, 4H). ¹³C NMR (100 MHz, DMSO-d₆) δ 170.3 (2C), 162.6 (2C), 160.6 (2C), 157.9 (2C), 157.4 (2C), 138.5 (2C), 128.9 (2C), 128.7 (4C), 127.9 (4C), 127.3 (4C), 125.7 (2C), 111.7 (4C), 108.5 (2C), 101.3 (2C), 93.3 (2C), 91.7 (2C), 88.4 (2C), 78.9 (2C), 69.8 (2C), 69.7 (2C), 69.6 (2C), 69.1 (2C), 55.4 (2C), 55.4 (2C), 55.3 (2C), 54.7 (2C), 50.0 (2C), 38.6 (2C). HRMS (m z): calculated for C₆₄H₇₃N₂O₁₈⁺ [M+H]⁺ 1157.4853, found 1157.4843. TLC: R_{f}=0.3 (10% methanol-dichloromethane).

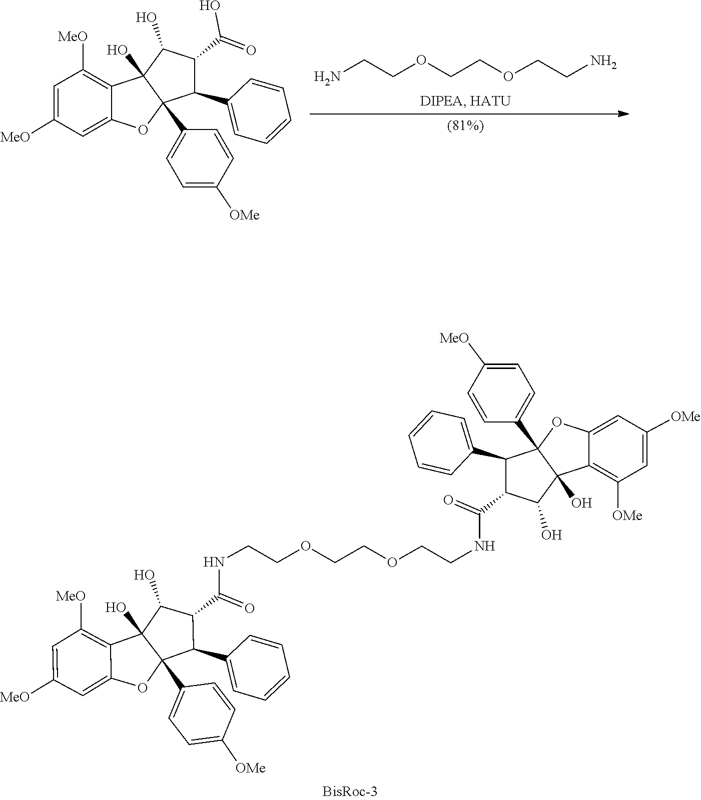 [SMILES: COc1ccc([C@@]23Oc4cc(OC)cc(OC)c4[C@]2(O)[C@H](O)[C@H](C(=O)O)[C@H]3c2ccccc2)cc1 ; NCCOCCOCCN ; COc1ccc([C@@]23Oc4cc(OC)cc(OC)c4[C@]2(O)[C@H](O)[C@H](C(=O)NCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

BisRoc-3. The same procedure as for BisRoc-1, using amino-PEG2-amine as starting material with scaled reagents, afforded BisRoc-3 (27 mg, 0.0253 mmol, 81% yield) as a white solid.

¹H NMR (400 MHz, DMSO-d₆) δ 8.47-8.11 (m, 2H), 7.13-6.99 (m, 8H), 6.99-6.89 (m, 6H), 6.59 (d, J=9.0 Hz, 4H), 6.27 (d, J=2.0 Hz, 2H), 6.10 (d, J=2.0 Hz, 2H), 4.96 (s, 2H), 4.54 (d, J=5.3 Hz, 4H), 4.18 (d, J=14.1 Hz, 2H), 3.85 (dd, J=14.1, 5.2 Hz, 2H), 3.78 (s, 6H), 3.72 (s, 6H), 3.60 (s, 6H), 3.43 (s, 4H), 3.39-3.26 (m, 4H), 3.22-3.09 (m, 4H). ¹³C NMR (100 MHz, DMSO-d₆) δ 170.3 (2C), 162.6 (2C), 160.6 (2C), 157.9 (2C), 157.4 (2C), 138.4 (2C), 128.8 (2C), 128.7 (4C), 127.9 (4C), 127.3 (4C), 125.7 (2C), 111.7 (4C), 108.5 (2C), 101.3 (2C), 93.3 (2C), 91.7 (2C), 88.4 (2C), 78.9 (2C), 69.5 (2C), 69.1 (2C), 55.5 (2C), 55.4 (2C), 55.3 (2C), 54.7 (2C), 50.0 (2C), 38.6 (2C). HRMS (m z): calculated for C₆₀H₆₅N₂O₁₆⁺ [M+H]⁺ 1069.4329, found 1069.4337. TLC: R_{f}=0.3 (10% methanol-dichloromethane).

What is claimed is:

1. A compound comprising a first eIF4A inhibitor attached to a second eIF4A inhibitor through a covalent linker.

2. The compound of claim 1, wherein the first eIF4A inhibitor and the second eIF4A inhibitor are the same.

3. The compound of claim 2, wherein the first eIF4A inhibitor and the second eIF4A inhibitor are a Rocaglate.

4. The compound of claim 2, wherein the first eIF4A inhibitor and the second eIF4A inhibitor are Rocaglamide A.

5. The compound of claim 2, wherein the first eIF4A inhibitor and the second eIF4A inhibitor are Zotatifin.

6. The compound of claim 2, wherein the first eIF4A inhibitor and the second eIF4A inhibitor are Silvestrol.

7. The compound of claim 2, wherein the first eIF4A inhibitor and the second eIF4A inhibitor are Pateamine A.

8. The compound of claim 2, wherein the first eIF4A inhibitor and the second eIF4A inhibitor are Hippuristanol.

9. The compound of claim 2, wherein the first eIF4A inhibitor and the second eIF4A inhibitor are a monovalent form of formula (I): 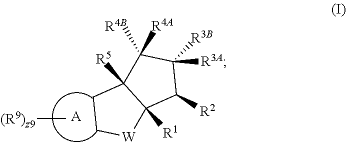
wherein
Ring A is aryl or heteroaryl;
W is CR⁶R⁷, O, S, NR⁸, C(O), C═CR⁶R⁷, N(CO)R⁸, S(O), or S(O)₂;
R¹ and R² are independently substituted or unsubstituted cycloalkyl, substituted or unsubstituted heterocycloalkyl, substituted or unsubstituted aryl, or substituted or unsubstituted heteroaryl;
R^{3A}, R^{3B}, R^{4A}, R^{4B}, R⁵, R⁶, and R⁷ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B}, or R^{4A} and R^{4B} independently combine to form oxo or substituted or unsubstituted alkenyl, substituted or unsubstituted cycloalkyl, or substituted or unsubstituted heterocycloalkyl;
R² and R^{3A}, R^{3A} and R^{4A}, R^{3B} and R^{4B}, or R^{4A} and R⁵, together with the carbon atom to which they are attached, form a substituted or unsubstituted cycloalkyl or substituted or unsubstituted heterocycloalkyl;
R⁸ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂ F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NR^{9C}NR^{9A}R^{9B}, —ONR^{9A}R^{9B}, —NHC(O)NR^{9A}R^{9B}, —N(O)ₘ₉, —NR^{9A}R^{9B}, —C(O)R^{9C}, —C(O)OR^{9C}, —C(O)NR^{9A}R^{9B}, —OR^{9D}, —SR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
X⁹ is independently —F, —Cl, —Br, or —I;
n9 is an integer from 0 to 4;
m9 and v9 are independently 1 or 2; and
z9 is an integer from 0 to 4.

10. The compound of claim 9, wherein R^{3A} is hydrogen.

11. The compound of claim 9, wherein R^{3B} is —C(O)NH₂ or substituted or unsubstituted 2 to 6 membered heteroalkyl.

12. The compound of claim 9, wherein R^{3B} is —C(O)NH₂ or —C(O)N(CH₃)₂.

13. The compound of claim 9, having the formula: 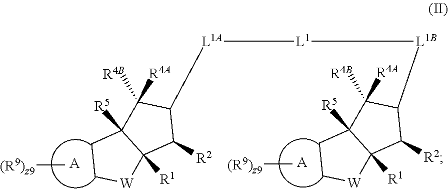
wherein
L¹ is said covalent linker;
L^{1A} is a bond, —C(O)—, —C(O)O—, —OC(O)—, —O—, —S—, —NR^{10A}—, —C(O)NR^{10A}—, —NR^{10A}C(O)—, —NR^{10A}C(O)O—, —OC(O)NR^{10A}—, —NR^{10A}C(O)NR^{10A}—, —NR^{10A}C(NH)NR^{10A}—, —S(O)₂—, —NR^{10A}S(O)₂—, —S(O)₂NR^{10A}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{1B} is a bond, —C(O)—, —C(O)O—, —OC(O)—, —O—, —S—, —NR^{10B}—, —C(O)NR^{101B}—, —NR^{10B}C(O)—, —NR^{10B}C(O)O—, —OC(O)NR^{10B}—, —NR^{10B}C(O)NR^{10B}—, —NR^{10B}C(NH)NR^{10B}, —S(O)₂—, —NR^{10B}S(O)₂—, —S(O)₂NR^{10B}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R^{10A} and R^{10B} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

14. The compound of claim 13, wherein L^{1A} is —C(O)NR^{10A}— or substituted or unsubstituted 2 to 6 membered heteroalkylene.

15. The compound of claim 13, wherein L^{1A} is —C(O)NR^{10A}—, —C(O)NR^{10A}CH₂—, or —CH₂NHC(O)—.

16. The compound of claim 13, wherein L^{1A} is —C(O)NR^{10A}—.

17. The compound of claim 14, wherein R^{10A} is hydrogen or unsubstituted C₁-C₄ alkyl.

18. The compound of claim 14, wherein R^{10A} is hydrogen.

19. The compound of claim 13, wherein L^{1B} is —NR^{10B}C(O)— or substituted or unsubstituted 2 to 6 membered heteroalkylene.

20. The compound of claim 13, wherein L^{1B} is —NR^{10B}C(O)—, —CH₂NR^{10A}C(O)—, or —C(O)NHCH₂—.

21. The compound of claim 13, wherein L^{1B} is —NR^{10B}C(O)—.

22. The compound of claim 19, wherein R^{10B} is hydrogen or unsubstituted C₁-C₄ alkyl.

23. The compound of claim 19, wherein R^{10B} is hydrogen.

24. The compound of claim 9, wherein Ring A is phenyl or a 5 to 6 membered heteroaryl.

25. The compound of claim 9, wherein Ring A is phenyl.

26. The compound of claim 9, wherein Ring A is pyridyl.

27. The compound of claim 9, wherein W is O, S, NH, or C(O).

28. The compound of claim 9, wherein W is O.

29. The compound of claim 9, wherein R¹ and R² are independently substituted or unsubstituted phenyl.

30. The compound of claim 9, wherein R^{4A} is hydrogen.

31. The compound of claim 9, wherein R^{4B} is —OH.

32. The compound of claim 9, wherein R⁵ is —OH.

33. The compound of claim 9, wherein R⁹ is —OR^{9D}.

34. The compound of claim 9, wherein R⁹ is —OCH₃.

35. The compound of claim 9, wherein z9 is 2.

36. The compound of claim 1, having the formula: 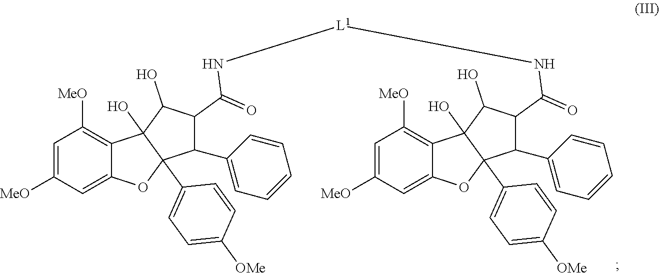
wherein
L¹ is said covalent linker.

37. The compound of claim 1, having the formula: 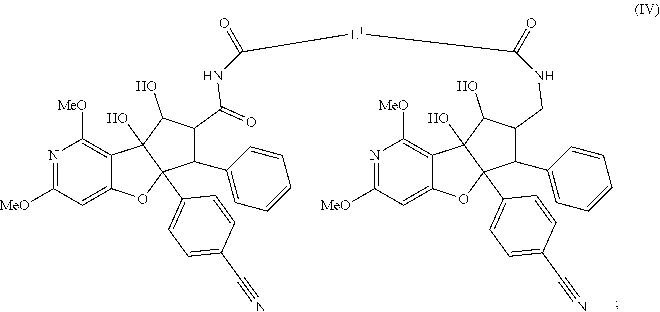
wherein
L¹ is said covalent linker.

38. The compound of claim 1, wherein L¹ is said covalent linker; and
L¹ is -L¹⁰¹-L¹⁰²-L¹⁰³-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², and R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

39. The compound of claim 38, wherein L¹ is 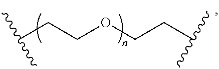
wherein n is an integer from 1 to 50.

40. The compound of claim 38, wherein L¹ is 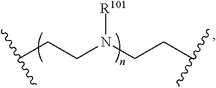
wherein R¹⁰¹ is independently hydrogen or unsubstituted C₁-C₄ alkyl; and n is an integer from 1 to 50.

41. The compound of claim 38, wherein L¹ is 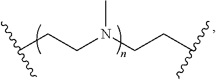
wherein n is an integer from 1 to 50.

42. The compound of claim 38, wherein L¹ is 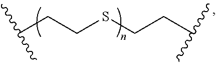
wherein n is an integer from 1 to 50.

43. The compound of claim 38, wherein L¹ is 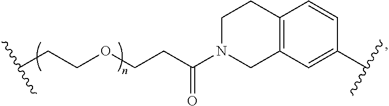
wherein n is an integer from 1 to 50.

44. The compound of claim 38, wherein L¹ is 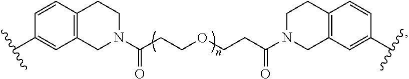
wherein nis an integer from 1 to 50.

45. The compound of claim 39, wherein n is an integer from 1 to 20.

46. The compound of claim 38, wherein L¹ is unsubstituted C₂-C₄₀ alkylene.

47. The compound of claim 1, having the formula:

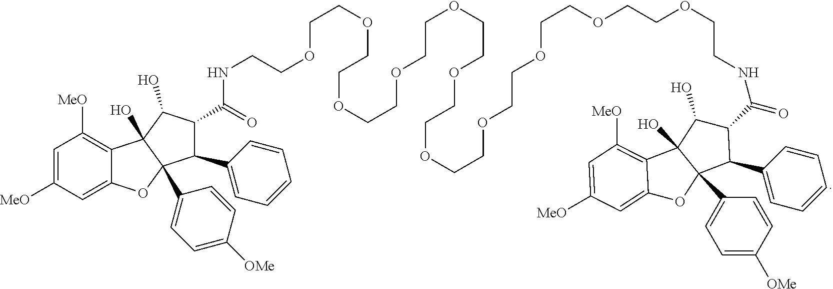 [SMILES: COc1ccc([C@@]23Oc4cc(OC)cc(OC)c4[C@]2(O)[C@H](O)[C@H](C(=O)NCCOCCOCCOCCOCCOCCOCCOCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

48. The compound of claim 1, having the formula:

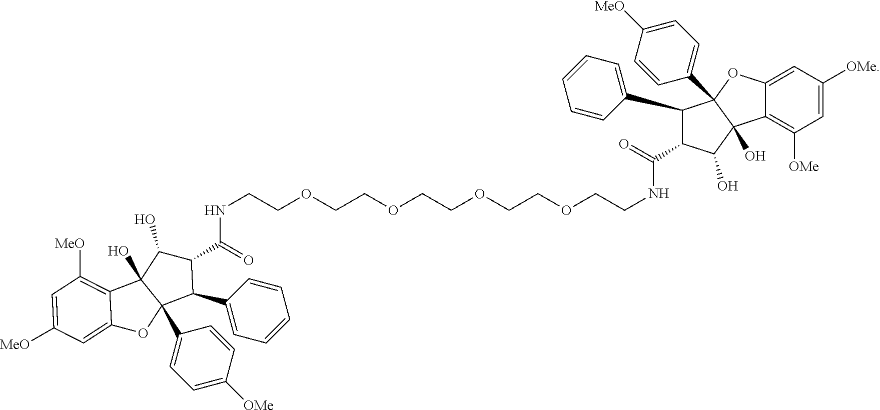 [SMILES: COc1ccc([C@@]23Oc4cc(OC)cc(OC)c4[C@]2(O)[C@H](O)[C@H](C(=O)NCCOCCOCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

49. The compound of claim 1, having the formula:

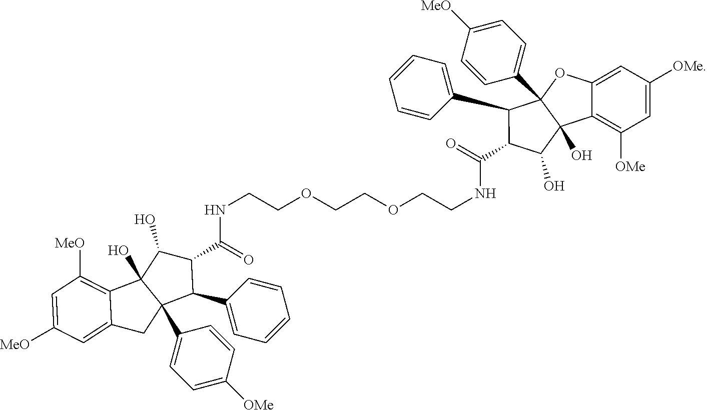 [SMILES: COc1ccc([C@@]23Cc4cc(OC)cc(OC)c4[C@]2(O)[C@H](O)[C@H](C(=O)NCCOCCOCCNC(=O)[C@H]2[C@@H](O)[C@@]4(O)c5c(OC)cc(OC)cc5O[C@@]4(c4ccc(OC)cc4)[C@@H]2c2ccccc2)[C@H]3c2ccccc2)cc1]

50. A pharmaceutical composition comprising a pharmaceutically acceptable excipient and a compound of one of claims 1 to 49, or a pharmaceutically acceptable salt thereof.

51. A method of treating cancer in a subject in need thereof, said method comprising administering to the subject in need thereof a therapeutically effective amount of a compound of one of claims 1 to 49, or a pharmaceutically acceptable salt thereof.

52. The method of claim 51, wherein the cancer is a MYC-amplified cancer.

53. The method of claim 51, wherein the cancer is an EGFR-amplified cancer.

54. The method of claim 51, wherein the cancer is a HER2-amplified cancer or a HER3-amplified cancer.

55. The method of claim 51, wherein the cancer is an FGFR1-amplified cancer or an FGFR2-amplified cancer.

56. The method of claim 51, wherein the cancer is an RTK-amplified cancer or a mutated RTK cancer.

57. The method of claim 51, wherein the cancer is a mutated KRAS cancer.

58. The method of claim 51, wherein the cancer is a cyclin D1-amplified cancer, a cyclin D2-amplified cancer, or a cyclin D3-amplified cancer.

59. The method of claim 51, wherein the cancer is a cancer with high phosphorylation of 4EBP1 or 4EBP2.

60. The method of claim 51, wherein the cancer is a cancer with loss of function of TSC1 or TSC2.

61. The method of claim 51, wherein the cancer is a cancer with loss of PTEN.

62. The method of claim 51, wherein the cancer is bladder cancer, breast cancer, colorectal cancer, endometrial cancer, esophageal cancer, head and neck cancer, gastric cancer, glioblastoma, leukemia, liver cancer, lung cancer, lymphoma, multiple myeloma, neuroblastoma, ovarian cancer, pancreatic cancer, parathyroid adenoma, prostate cancer, renal cancer, skin cancer, thyroid cancer, or uterine cancer.

63. The method of claim 62, wherein the breast cancer is triple negative breast cancer.

64. The method of claim 62, wherein the esophageal cancer is esophageal adenocarcinoma.

65. The method of claim 62, wherein the gastric cancer is gastric adenocarcinoma.

66. The method of claim 62, wherein the leukemia is acute myeloid leukemia.

67. The method of claim 62, wherein the liver cancer is hepatocellular carcinoma.

68. The method of claim 62, wherein the lung cancer is non-small cell lung cancer, small cell lung cancer, or squamous cell lung carcinoma.

69. The method of claim 62, wherein the skin cancer is melanoma or squamous cell carcinoma.

70. A method of modulating the level of activity of an eIF4A protein in a cell, said method comprising contacting the cell with an effective amount of a compound of one of claims 1 to 49, or a pharmaceutically acceptable salt thereof.
